(19) United States
(12) Patent Application Publication
(10) Pub. No.: US 2006/0211697 A1
(43) Pub. Date: Sep. 21, 2006

(54) 2-(2 Or 4-substituted aryloxy)-phenol derivatives as antibacterial agents

(76) Inventors: Liren Huang, South Brunswick, NJ (US); Joanna Clancy, Rockville, MD (US); Alenka Tomazic, Ljubljana (SI); Weitong Wang, Germantown, MD (US); Christopher Taylor, Catonsville, MD (US); W. James Jackson, Marriottsville, MD (US)

Correspondence Address: MANELLI DENISON & SELTER, 2000 M STREET NW SUITE 700, WASHINGTON, DC, 20036-3307, US

(21) Appl. No.: 11/289,362

(22) Filed: Nov. 30, 2005

Related U.S. Application Data
(60) Provisional application No. 60/634,085, filed on Dec. 8, 2004.

(51) Int. Cl. C07D 271/06; C07D 285/12; C07D 277/20; C07D 275/02; C07D 263/02; A61K 31/50

(52) U.S. Cl. 514/247; 514/362; 514/364; 514/365; 514/374; 514/372; 514/378; 514/408; 514/381; 514/406; 514/396; 514/383; 514/438; 514/461; 544/224; 548/131; 548/136; 548/202; 548/206; 548/215; 548/252; 548/255; 548/263.2

(57) ABSTRACT

Antimicrobial compounds, compositions and methods of treatment administering same, of 2-aryloxyphenol derivatives having a heterocyclic or polar functional substitution attached through a N—C or C—C bond at the position para or ortho relative to oxygen bridge on non-phenolic phenyl ring, as well as methods for their preparation and formation, wherein the compounds are generally of Formula 1 and Formula 2:

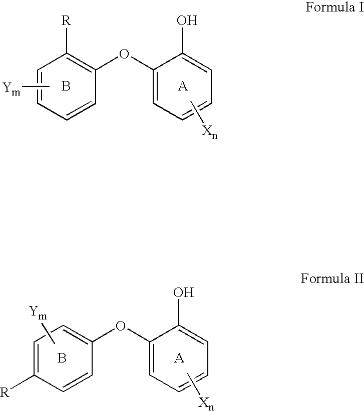

### FIELD OF THE INVENTION

This invention relates to novel substituted 2-aryloxyphenol derivatives possessing a heterocyclic or polar functional substitution attached through a N—C or C—C bond at the position para or ortho relative to oxygen bridge on non-phenolic phenyl ring, methods for the preparation of these compounds, and pharmaceutical compositions comprising the compounds. The compounds are useful antimicrobial agents, effective against a number of human and bioterrorism pathogens, including staphylococci, streptococci and enterococci, as well as Bacillus anthracis and Bacillus cereus.

### BACKGROUND OF THE INVENTION

Drug resistance of existing antimicrobial and particularly antibacterial agents is a clinical problem worldwide. A number of approaches have been taken by the pharmaceutical community to combat the alarming bacterial resistance problem. One approach is the structural modification of known antibiotics to overcome resistance liabilities. A second approach is combination therapies, for example, the combination of antibiotics with drugs that inhibit the enzyme or protein that causes a particular resistance. Although these approaches have met with some success, the best solution to the bacterial resistance dilemma remains the identification of novel antibacterial agents employing a unique mechanism of action.

The chemical and biological literature abounds with reports about 2-aryloxyphenols (A) due in great part to their antibacterial activities. Many of the compounds were initially used in the treatment of textiles, and there have been hundreds of patents filed worldwide for their incorporation into a diverse range of products over the last 30 years. Triclosan (B) is the most potent and widely used member of this class of antibacterial and antifungal agents, and is used in products such as antiseptic soaps, toothpastes, fabrics and plastics.

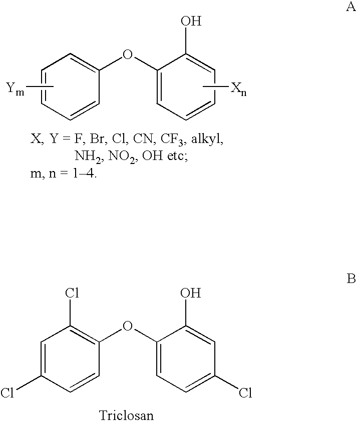

The U.S. Pat. No. 3,506,720, U.S. Pat. No. 3,903,007, NR 432119, DE 2800105 A1 and CH 460443 describe the usefulness of halogenated 2-aryloxyphenols, especially 5-chloro-(2,4-dichloro-phenoxy)-phenol (triclosan), and corresponding esters in antibacterial compositions and methods for the protection of organic materials, films and textile fibers.

The patent U.S. Pat. No. 5,185,377, U.S. Pat. No. 6,204,230, U.S. Pat. No. 6,107,261, U.S. Pat. No. 6,136,771 and WO 98/55096 describe the pharmaceutical compositions which comprise triclosan and other 2-aryloxyphenols useful in treatment of bacterial infections, inflammatory disease, and spasmolytic disease.

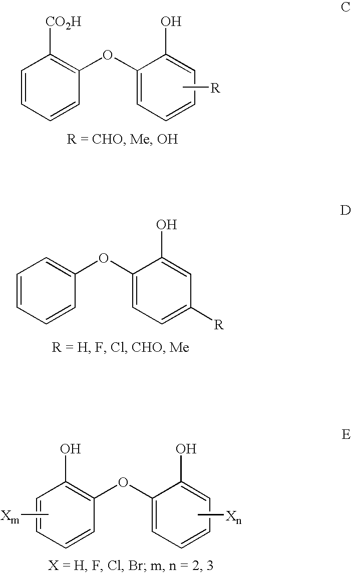

Syntheses of compounds of type C, where one of the benzene rings is substituted with a 2-carboxylic group, have been reported by Fujikawa (Yakugaku Zasshi (1963), 63, 1172) but there is no biological activity data reported. Health and Sivaraman have reported the relation of the antibacterial activities of triclosan and its related compounds D to inhibition of the bacterial enoyl reductase Fab I. (J. Bio. Chem., 1998, 273:3016; J. Med. Chem., 2004, 47:509). Studies on the synthesis, activity and molecular modeling of type E compounds as human aldose reductase inhibitors have been described. (J. Med. Chem., 2003, 46:5208) 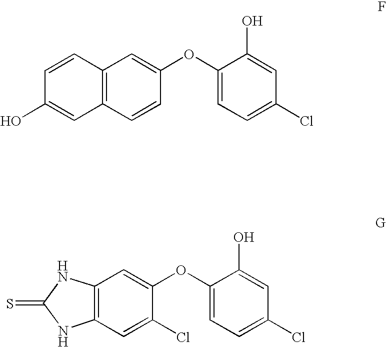
Antimalarial activities of triclosan and compound F associated with their inhibitory activity against malarial enoyl carrier protein reductase have been described by Perozzo, R. (J. Bio. Chem., 2002, 277:13106). The U.S. Pat. No. 4,205,077 described cyclic thiourea derivatives of 2-aryloxyphenol including compound G useful as anthelmintic agents in animals.

Triclosan had long been thought of as a nonspecific biocide that disrupts cell membranes, rendering bacteria unable to assimilate nutrients and to proliferate. This view has been changed recently by McMurry, Health et al who discovered that triclosan and other members of the 2-aryloxyphenols, such as compound D, directly target Fab I, the enoyl-acyl carrier protein reductase of type II bacterial fatty acid synthesis. (Nature, 1998, 394:531; J. Bio. Chem., 1998, 273:3016; J. Med. Chem., 2004, 47:509). This was followed by analyses of the crystal structure of the Escherichia coli Fab I—NAD+—triclosan complex and computational chemistry thereafter (Biochemistry, 2003, 42:4406; J. Bio. Chem., 2002, 277:13106; Protein Sci., 1999, 8:2529) laid the foundation for rational drug design in the area of 2-aryloxyphenol antibacterial agents.

The present invention includes the design and synthesis of novel 2-aryloxyphenol derivatives by incorporation of heterocyclic or highly polar functional groups in order to improve their water solubility, bio-availability, and microbial activity in vivo.

### DETAILED DESCRIPTION OF THE INVENTION

The present invention includes compounds and compositions of structural formula I and formula II or pharmaceutically acceptable salts thereof, 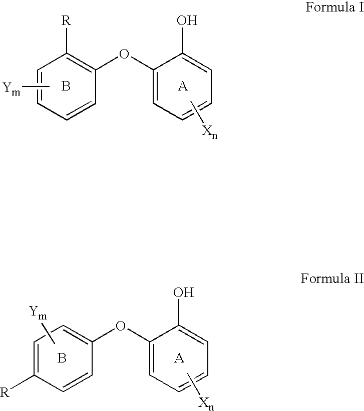
Wherein,
X and Y are each independently chosen from halogen, CN, OH, NH₂, NO₂, CO₂H, CONH₂, SO₃H, SO₂NH₂, CHO, CH(NOMe), C(O)Me, C₁-C₄ alkyl and cycloalkyl, and CF₃; m and n are 0, 1, 2 and 3;
R is chosen from C(NH)NH₂, C(NOH)NH₂, C(NNH₂)NH₂, C(O)NHOH, NHNH₂, NHC(O)H, NHC(NH)NH₂, NHSO₂Me, a heterocyclic group of 5-8 atoms with 1-4 heteroatoms chosen from nitrogen, oxygen, or sulfur or phenyl, all of which rings may be optionally substituted up to 3 times by halogen, OH, NH₂, NO₂, NHAc, methyl, ethyl, CHO, CN, CH₂OH, CO₂H, CONH₂, CO₂Me and CO₂Et.

More preferred compounds of the present invention are those of Formula I and Formula II wherein X and Y are independently F, Cl, Br, I, CN, OH, NH₂, NO₂, CONH₂, SO₂NH₂, CHO, CH(NOMe), methyl, ethyl, n-propyl, n-butyl, cyclopropyl, cycloproylmethyl and CF₃; m and n are 0, 1, 2 and 3;

R is C(NH)NH₂, C(NOH)NH₂, C(NNH₂)NH₂, C(O)NHOH, NHNH₂, NHC(O)H, NHC(NH)NH₂, NHSO₂Me, a heterocyclic group chosen from

(a) Substituted furanyl:

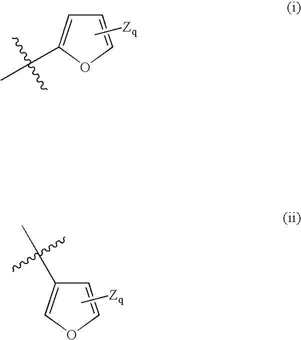

(b) Subtituted thiophenyl:

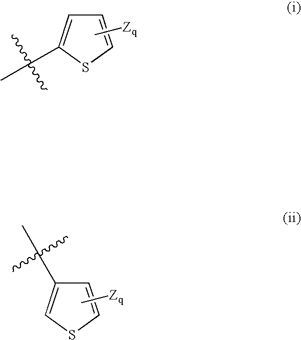

(c) Substituted pyrrolyl:

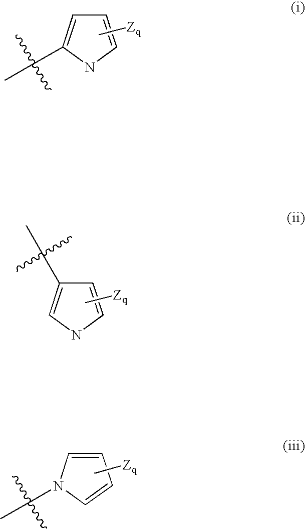

(d) Substituted isoxazolyl

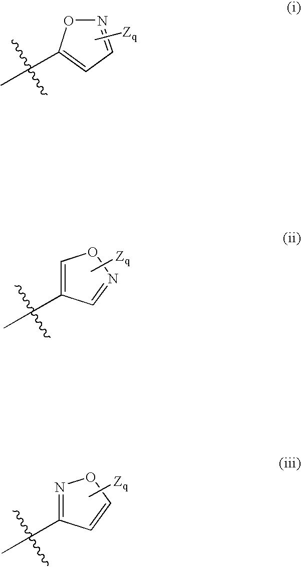

(e) Substituted isothiazolyl

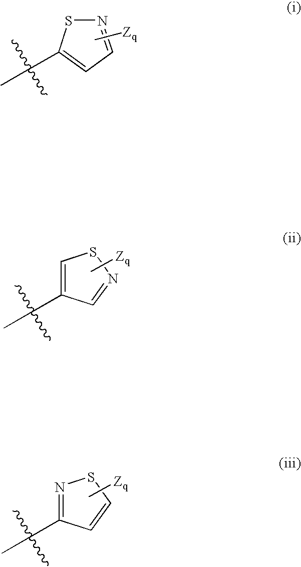

(f) Substituted pyrazolyl

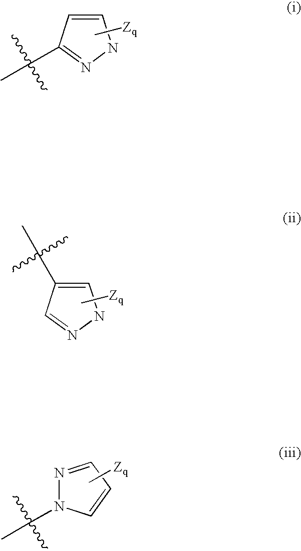

(g) Substituted oxazolyl

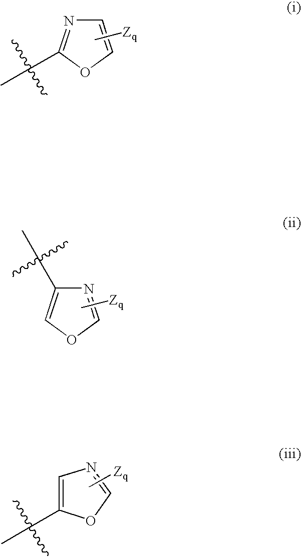

(h) Substituted thiazolyl

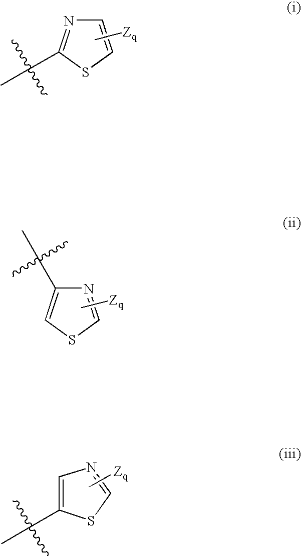

(i) Substituted imidazolyl

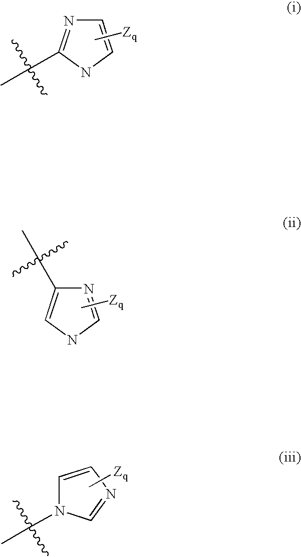

(j) Substituted 1H-[1,2,3]triazolyl

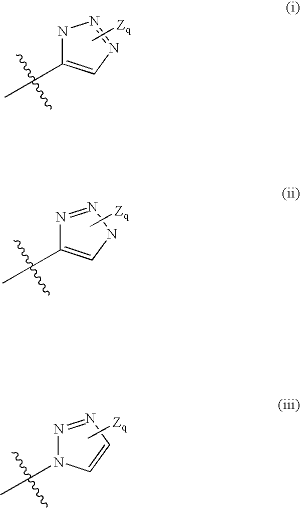

(k) Substituted 4H-[1,2,4]triazolyl

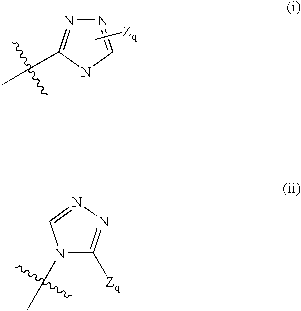

(l) Substituted 1H-[1,2,4]triazolyl

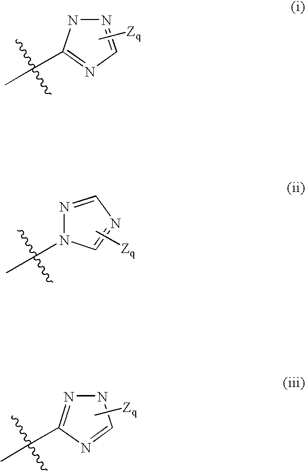

(m) Substituted [1,3,4]oxadiazolyl

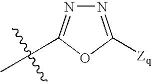

(n) Substituted [1,3,4]thiadiazolyl

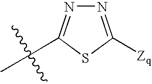

(o) Substituted [1,2,4]oxadiazolyl

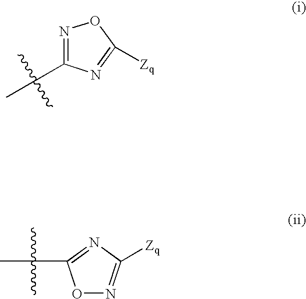

(p) 1H-tetrazol-5-yl (i) or 2H-tetrazol-5-yl

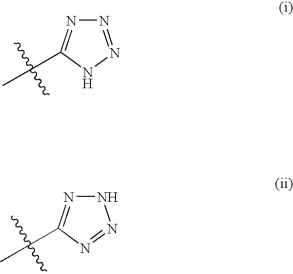

(q) 1H-tetrazol-1-yl

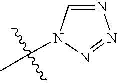

(r) 5-oxo-4H-[1,2,4]oxadiazol-3-yl

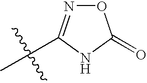

(s) Substituted thiazolidin-2-yl and [1,3]thiazinan-2-yl

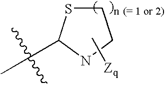

(t) Substituted 4,5-dihydro-thiazol-2-yl and 5,6-dihydro-4H-[1,3]thiazin-2-yl

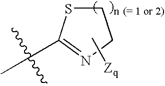

(u) Substituted pyridazinyl 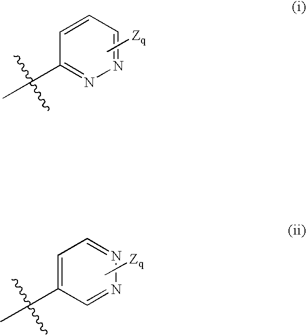
Wherein Z is independently chosen from OH, NH₂, NHAc, Me, Et, CHO, F, Cl, CN, CH₂OH, CO₂H, CONH₂, CO₂Me, CO₂Et; q is 0, 1, 2, 3.

More preferred compounds of the present invention include the compounds of Formula I and Formula II,

wherein

X and Y are independently Cl, F, Br, CN, methyl, ethyl, cyclopropyl, cyclopropylmethyl, CF₃, OH, NH₂, NO₂, CHO, SO₂NH₂; m and n are 0, 1, 2 and 3;

R is C(NH)NH₂, C(NOH)NH₂, C(NNH₂)NH₂, NHNH₂, NHC(O)H, NHC(NH)NH₂, NHSO₂Me, a heterocyclic group chosen from structural formula (a)-(i) and (ii), (b)-(i) and (ii), (c)-(i) and (iii), (f)-(i) and (iii), (i)-(i), (ii) and (iii), (j)-(i), (ii) and (iii), (k)-(i) and (ii), (l)-(i), (ii), and (iii), (m), (n), (O)-(i) and (ii), (p)-(i) and (ii), (q), (r), (s), (t) and (u), wherein Z is independently chosen from OH, NH₂, NHAc, methyl, ethyl, CHO, F, Cl, CN, CH₂OH, CO₂H, CONH₂, CO₂Me, CO₂Et; q is 0, 1, 2 and 3.

Even more preferably, in the present invention R is C(NH)NH₂, C(NOH)NH₂, C(NNH₂)NH₂, NHNH₂, NHC(O)H, NHSO₂Me, a heterocyclic group chosen from structural formula (a)-(i), (b)-(i) and (ii), (c)-(iii), (f)-(i) and (iii), (j)-(i), (ii) and (iii), (l)-(i), (ii), and (iii), (m), (n), (O)-(i), (p)-(i) and (ii), (q), (r), (s) and (t), wherein Z is independently chosen from OH, NH₂, methyl, ethyl, CN, CO₂H, CO₂Me, CO₂Et; q is 0, 1 and 2.

The present invention includes pharmaceutical compositions which comprise an antibacterially effective amount of compounds of structural Formula I and Formula II or pharmaceutically acceptable salts thereof with pharmaceutical acceptable carriers.

Preferred Compounds

The compounds of the present invention listed below are preferred:

1. 2-(2-Hydroxy-phenoxy)-5-(thiophen-3-yl)-phenol;
2. 2-(2-Hydroxy-4-methyl-phenoxy)-5-(thiophen-3-yl)-phenol;
3. 2-(2-Hydroxy-4-methyl-phenoxy)-5-(thiophen-2-yl)-phenol;
4. 2-(2-Hydroxy-4-methyl-phenoxy)-5-(furan-2-yl)-phenol;
5. 5-Methyl-2-[4-(thiazolidin-2-yl)-phenoxy]-phenol;
6. 2-[4-(2-Hydroxy-4-methyl-phenoxy)-phenyl]-thiazolidine-4-carboxylic acid methyl ester;
7. 2-[4-(2-Hydroxy-4-methyl-phenoxy)-phenyl]-thiazolidine-4-carboxylic acid;
8. 2-[4-(2-Hydroxy-4-methyl-phenoxy)-phenyl]-[1, 3]thiazinane-4-carboxylic acid;
9. 2-[3-Cyano-4-(2-hydroxy-4-methyl-phenoxy)-phenyl]-thiazolidine-4-carboxylic acid;
10. 2-[3-Cyano-4-(2-hydroxy-4-methyl-phenoxy)-phenyl]-[1, 3]thiazinane-4-carboxylic acid;
11. 5-Methyl-2-[4-(pyrrol-1-yl)-phenoxy]-phenol;
12. 5-(Furan-2-yl)-2-(4-fluoro-2-hydroxy-phenoxy)-phenol;
13. 5-Methyl-2-[4-(1H-tetrazol-5-yl)-phenoxy]-phenol and 5-methyl-2-[4-(2H-tetrazol-5-yl)-phenoxy]-phenol;
14. 5-Methyl-2-[2-(1H-tetrazol-5-yl)-phenoxy]-phenol and 5-methyl-2-[2-(2H-tetrazol-5-yl)-phenoxy]-phenol;
15. 5-Chloro-2-[2-(1H-tetrazol-5-yl)-phenoxy]-phenol and 5-chloro-2-[2-(2H-tetrazol-5-yl)-phenoxy]-phenol;
16. 5-Methyl-2-[4-nitro-2-(1H-tetrazol-5-yl)-phenoxy]-phenol and 5-methyl-2-[4-nitro-2-(2H-tetrazol-5-yl)-phenoxy]-phenol;
17. 5-Fluoro-2-[2-(1H-tetrazol-5-yl)-phenoxy]-phenol and 5-fluoro-2-[2-(2H-tetrazol-5-yl)-phenoxy]-phenol;
18. 2-[4-Chloro-2-(1H-tetrazol-5-yl)-phenoxy]-5-fluoro-phenol and 2-[4-chloro-2-(2H-tetrazol-5-yl)-phenoxy]-5-fluoro-phenol;
19. 5-chloro-2-[4-Chloro-2-(1H-tetrazol-5-yl)-phenoxy]-phenol and 5-chloro-2-[4-chloro-2-(2H-tetrazol-5-yl)-phenoxy]-phenol;
20. 2-[2-(1H-Tetrazol-5-yl)-phenoxy]-phenol and 2-[2-(2H-tetrazol-5-yl)-phenoxy]-phenol;
21. 4-Bromo-2-[2-(1H-tetrazol-5-yl)-phenoxy]-phenoll and 4-bromo-2-[2-(2H-tetrazol-5-yl)-phenoxy]-phenol;
22. 2-[2-(5-Amino-[1,3,4]thiadiazol-2-yl)-phenoxy]-5-fluoro-phenol;
23. 2-[4-(5-Amino-[1,3,4]thiadiazol-2-yl)-phenoxy]-5-methyl-phenol;
24. 2-[4-(5-Amino-[1,3,4]oxadiazol-2-yl)-phenoxy]-5-methyl-phenol;
25. 2-(4-Fluoro-2-hydroxy)-N-hydroxy-benzamidine;
26. N-Hydroxy-2-(2-hydroxy-4-methyl-phenoxy)-benzamidine;
27. 5-Chloro-2-(4-fluoro-2-hydroxy)-N-hydroxy-benzamidine;
28. N-Hydroxy-2-(2-hydroxy-4-methyl-phenoxy)-5-nitro-benzamidine;
29. 5-Chloro-2-(4-chloro-2-hydroxy-phenoxy)-N-hydroxy-benzamidine;
30. N-Hydroxy-4-(2-hydroxy-4-methyl-phenoxy)-benzamidine;
31. 3-[2-(4-Fluoro-2-hydroxy)-phenyl]-[1,2,4]oxadiazole-5-carboxylic acid ethyl ester;
32. 3-[2-(2-Hydroxy-4-methyl-phenoxy)-phenyl]-[1,2,4]oxadiazole-5-carboxylic acid ethyl ester;
33. 3-[5-Chloro-2-(4-fluoro-2-hydroxy-phenoxy)-phenyl]-[1,2,4]oxadiazole-5-carboxylic acid ethyl ester;
34. 3-[2-(2-Hydroxy-4-methyl-phenoxy)-5-nitro-phenyl]-[1,2,4]oxadiazole-5-carboxylic acid ethyl ester;
35. 3-[4-(2-Hydroxy-4-methyl-phenoxy)-phenyl]-[1,2,4]oxadiazole-5-carboxylic acid ethyl ester;
36. 3-[2-(4-Fluoro-2-hydroxy-phenoxy)-phenyl]-[1,2,4]oxadiazole-5-carboxylic acid;
37. 3-[2-(2-Hydroxy-4-methyl-phenoxy)-phenyl]-[1,2,4]oxadiazole-5-carboxylic acid
38. 3-[5-Chloro-2-(4-fluoro-2-hydroxy-phenoxy)-phenyl]-[1,2,4]oxadiazole-5-carboxylic acid;
39. 3-[2-(2-Hydroxy-4-methyl-phenoxy)-5-nitro-phenyl]-[1,2,4]oxadiazole-5-carboxylic acid;
40. 3-[4-(2-Hydroxy-4-methyl-phenoxy)-phenyl]-[1,2,4]oxadiazole-5-carboxylic acid;
41. 5-Methyl-2-(4-nitro-2-[1,2,4]oxadiazol-3-yl-phenoxy)-phenol;
42. 5-Fluoro-2-(2-[1,2,4]oxadiazol-3-yl-phenoxy)-phenol;
43. 5-Methyl-2-(2-[1,2,4]oxadiazol-3-yl-phenoxy)-phenol;
44. 2-(4-Chloro-2-[1,2,4]oxadiazol-3-yl-phenoxy)-5-fluoro-phenol;
45. 5-Methyl-2-(4-[1,2,4]oxadiazol-3-yl-phenoxy)-phenol;
46. 3-[2-(2-Hydroxy-4-methyl-phenoxy)-phenyl]-4H-[1,2,4]oxadiazol-5-one;
47. 3-[4-(2-Hydroxy-4-methyl-phenoxy)-phenyl]-4H-[1,2,4]oxadiazol-5-one;
48. 2-(4-Chloro-2-hydroxy-phenoxy)-N-hydroxy-benzamidine;
49. N-Hydroxy-4-(2-hydroxy-phenoxy)-3-nitro-benzamidine;
50. 4-(4-Chloro-2-hydroxy-phenoxy)-N-hydroxy-3-nitro-benzamidine;
51. 3-[2-(4-Chloro-2-hydroxy-phenoxy)-phenyl]-[1,2,4]oxadiazole-5-carboxylic acid ethyl ester;
52. 3-[4-(2-Hydroxy-phenoxy)-3-nitro-phenyl]-[1,2,4]oxadiazole-5-carboxylic acid ethyl ester;
53. 3-[4-(4-Chloro-2-hydroxy-phenoxy)-3-nitro-phenyl]-[1,2,4]oxadiazole-5-carboxylic acid ethyl ester;
54. 3-[2-(4-Chloro-2-hydroxy-phenoxy)-phenyl]-[1,2,4]oxadiazole-5-carboxylic acid;
55. 3-[4-(2-Hydroxy-phenoxy)-3-nitro-phenyl]-[1,2,4]oxadiazole-5-carboxylic acid;
56. 3-[4-(4-Chloro-2-hydroxy-phenoxy)-3-nitro-phenyl]-[1,2,4]oxadiazole-5-carboxylic acid;
57. 5-Chloro-2-(2-[1,2,4]oxadiazol-3-yl-phenoxy)-phenol;
58. 5-Methyl-2-(2-tetrazol-1-yl-phenoxy)-phenol;
59. 5-Methyl-2-(4-tetrazol-1-yl-phenoxy)-phenol;
60. N-Amino-4-(2-hydroxy-4-methyl-phenoxy)-benzamidine;
61. N-Amino-2-(2-hydroxy-4-methyl-phenoxy)-benzamidine;
62. 1-[2-(2-Hydroxy-4-methyl-phenoxy)-phenyl]-1H-[1,2,3]triazole-4-carboxylic acid ethyl ester;
63. 1-[4-(2-Hydroxy-4-methyl-phenoxy)-phenyl]-1H-[1,2,3]triazole-4-carboxylic acid ethyl ester;
64. 1-[2-(2-Hydroxy-4-methyl-phenoxy)-phenyl]-1H-[1,2,3]triazole-4-carboxylic acid;
65. 1-[4-(2-Hydroxy-4-methyl-phenoxy)-phenyl]-1H-[1,2,3]triazole-4-carboxylic acid;
66. 2-(2-Hydrazino-phenoxy)-5-methyl-phenol;
67. 2-(4-Hydrazino-phenoxy)-5-methyl-phenol;
68. 1-[2-(2-Hydroxy-4-methyl-phenoxy)-phenyl]-1H-pyrazole-4-carboxylic acid ethyl ester;
69. 1-[4-(2-Hydroxy-4-methyl-phenoxy)-phenyl]-1H-pyrazole-4-carboxylic acid ethyl ester;
70. 1-[2-(2-Hydroxy-4-methyl-phenoxy)-phenyl]-1H-pyrazole-4-carboxylic acid;
71. 1-[4-(2-Hydroxy-4-methyl-phenoxy)-phenyl]-1H-pyrazole-4-carboxylic acid;
72. 5-Methyl-2-(2-[1,2,4]triazol-1-yl-phenoxy)-phenol;
73. 5-Methyl-2-(4-[1,2,4]triazol-1-yl-phenoxy)-phenol;
74. 5-Amino-1-[2-(2-hydroxy-4-methyl-phenoxy)-phenyl]-1H-pyrazole-4-carboxylic acid ethyl ester;
75. 5-Amino-1-[4-(2-hydroxy-4-methyl-phenoxy)-phenyl]-1H-pyrazole-4-carboxylic acid ethyl ester;
76. 2-[2-(5-Amino-3-methylsulfanyl-[1,2,4]triazol-1-yl)-phenoxy]-5-methyl-phenol.
   Most Preferred Compounds

The compounds of the present invention listed below are most preferred:
1. 2-(2-Hydroxy-phenoxy)-5-(thiophen-3-yl)-phenol;
2. 2-(2-Hydroxy-4-methyl-phenoxy)-5-(thiophen-3-yl)-phenol;
3. 2-(2-Hydroxy-4-methyl-phenoxy)-5-(thiophen-2-yl)-phenol;
4. 2-(2-Hydroxy-4-methyl-phenoxy)-5-(furan-2-yl)-phenol;
5. 5-Methyl-2-[4-(thiazolidin-2-yl)-phenoxy]-phenol;
6. 2-[4-(2-Hydroxy-4-methyl-phenoxy)-phenyl]-thiazolidine-4-carboxylic acid methyl ester;
7. 2-[4-(2-Hydroxy-4-methyl-phenoxy)-phenyl]-thiazolidine-4-carboxylic acid;
8. 2-[4-(2-Hydroxy-4-methyl-phenoxy)-phenyl]-[1, 3]thiazinane-4-carboxylic acid;
9. 2-[3-Cyano-4-(2-hydroxy-4-methyl-phenoxy)-phenyl]-thiazolidine-4-carboxylic acid;
10. 2-[3-Cyano-4-(2-hydroxy-4-methyl-phenoxy)-phenyl]-[1, 3]thiazinane-4-carboxylic acid;
11. 5-(Furan-2-yl)-2-(4-fluoro-2-hydroxy-phenoxy)-phenol;
12. 5-Methyl-2-[4-(1H-tetrazol-5-yl)-phenoxy]-phenol and 5-methyl-2-[4-(2H-tetrazol-5-yl)-phenoxy]-phenol;
13. 5-Methyl-2-[2-(1H-tetrazol-5-yl)-phenoxy]-phenol and 5-methyl-2-[2-(2H-tetrazol-5-yl)-phenoxy]-phenol;
14. 5-Chloro-2-[2-(1H-tetrazol-5-yl)-phenoxy]-phenol and 5-chloro-2-[2-(2H-tetrazol-5-yl)-phenoxy]-phenol;
15. 5-Methyl-2-[4-nitro-2-(1H-tetrazol-5-yl)-phenoxy]-phenol and 5-methyl-2-[4-nitro-2-(2H-tetrazol-5-yl)-phenoxy]-phenol;
16. 5-Fluoro-2-[2-(1H-tetrazol-5-yl)-phenoxy]-phenol and 5-fluoro-2-[2-(2H-tetrazol-5-yl)-phenoxy]-phenol;
17. 2-[4-Chloro-2-(1H-tetrazol-5-yl)-phenoxy]-5-fluoro-phenol and 2-[4-chloro-2-(2H-tetrazol-5-yl)-phenoxy]-5-fluoro-phenol;
18. 5-chloro-2-[4-Chloro-2-(1H-tetrazol-5-yl)-phenoxy]-phenol and 5-chloro-2-[4-chloro-2-(2H-tetrazol-5-yl)-phenoxy]-phenol;
19. 2-(4-Fluoro-2-hydroxy)-N-hydroxy-benzamidine,
20. N-Hydroxy-2-(2-hydroxy-4-methyl-phenoxy)-benzamidine;
21. 5-Chloro-2-(4-fluoro-2-hydroxy)-N-hydroxy-benzamidine;
22. N-Hydroxy-2-(2-hydroxy-4-methyl-phenoxy)-5-nitro-benzamidine;
23. 5-Chloro-2-(4-chloro-2-hydroxy-phenoxy)-N-hydroxy-benzamidine;
24. N-Hydroxy-4-(2-hydroxy-4-methyl-phenoxy)-benzamidine;
25. 3-[2-(4-Fluoro-2-hydroxy)-phenyl]-[1,2,4]oxadiazole-5-carboxylic acid ethyl ester;
26. 3-[2-(2-Hydroxy-4-methyl-phenoxy)-phenyl]-[1,2,4]oxadiazole-5-carboxylic acid ethyl ester;
27. 3-[5-Chloro-2-(4-fluoro-2-hydroxy-phenoxy)-phenyl]-[1,2,4]oxadiazole-5-carboxylic acid ethyl ester;
28. 3-[2-(2-Hydroxy-4-methyl-phenoxy)-5-nitro-phenyl]-[1,2,4]oxadiazole-5-carboxylic acid ethyl ester;
29. 3-[4-(2-Hydroxy-4-methyl-phenoxy)-phenyl]-[1,2,4]oxadiazole-5-carboxylic acid ethyl ester;
30. 3-[2-(4-Fluoro-2-hydroxy-phenoxy)-phenyl]-[1,2,4]oxadiazole-5-carboxylic acid;
31. 3-[2-(2-Hydroxy-4-methyl-phenoxy)-phenyl]-[1,2,4]oxadiazole-5-carboxylic acid;
32. 3-[5-Chloro-2-(4-fluoro-2-hydroxy-phenoxy)-phenyl]-[1,2,4]oxadiazole-5-carboxylic acid;
33. 3-[2-(2-Hydroxy-4-methyl-phenoxy)-5-nitro-phenyl]-[1,2,4]oxadiazole-5-carboxylic acid;
34. 3-[4-(2-Hydroxy-4-methyl-phenoxy)-phenyl]-[1,2,4]oxadiazole-5-carboxylic acid;
35. 5-Methyl-2-(4-nitro-2-[1,2,4]oxadiazol-3-yl-phenoxy)-phenol;
36. 5-Fluoro-2-(2-[1,2,4]oxadiazol-3-yl-phenoxy)-phenol;
37. 5-Methyl-2-(2-[1,2,4]oxadiazol-3-yl-phenoxy)-phenol;
38. 2-(4-Chloro-2-[1,2,4]oxadiazol-3-yl-phenoxy)-5-fluoro-phenol;
39. 5-Methyl-2-(4-[1,2,4]oxadiazol-3-yl-phenoxy)-phenol;
40. 2-(4-Chloro-2-hydroxy-phenoxy)-N-hydroxy-benzamidine;
41. 4-(4-Chloro-2-hydroxy-phenoxy)-N-hydroxy-3-nitro-benzamidine;
42. 3-[2-(4-Chloro-2-hydroxy-phenoxy)-phenyl]-[1,2,4]oxadiazole-5-carboxylic acid ethyl ester;
43. 3-[4-(2-Hydroxy-phenoxy)-3-nitro-phenyl]-[1,2,4]oxadiazole-5-carboxylic acid ethyl ester;
44. 3-[4-(4-Chloro-2-hydroxy-phenoxy)-3-nitro-phenyl]-[1,2,4]oxadiazole-5-carboxylic acid ethyl ester;
45. 3-[2-(4-Chloro-2-hydroxy-phenoxy)-phenyl]-[1,2,4]oxadiazole-5-carboxylic acid;
46. 3-[4-(2-Hydroxy-phenoxy)-3-nitro-phenyl]-[1,2,4]oxadiazole-5-carboxylic acid;
47. 3-[4-(4-Chloro-2-hydroxy-phenoxy)-3-nitro-phenyl]-[1,2,4]oxadiazole-5-carboxylic acid;
48. 5-Chloro-2-(2-[1,2,4]oxadiazol-3-yl-phenoxy)-phenol;
49. 5-Methyl-2-(2-tetrazol-1-yl-phenoxy)-phenol;
50. 5-Methyl-2-(4-tetrazol-1-yl-phenoxy)-phenol;
51. N-Amino-4-(2-hydroxy-4-methyl-phenoxy)-benzamidine;
52. N-Amino-2-(2-hydroxy-4-methyl-phenoxy)-benzamidine;
53. 1-[2-(2-Hydroxy-4-methyl-phenoxy)-phenyl]-1H-[1,2,3]triazole-4-carboxylic acid ethyl ester;
54. 1-[4-(2-Hydroxy-4-methyl-phenoxy)-phenyl]-1H-[1,2,3]triazole-4-carboxylic acid ethyl ester;
55. 1-[2-(2-Hydroxy-4-methyl-phenoxy)-phenyl]-1H-[1,2,3]triazole-4-carboxylic acid;
56. 1-[4-(2-Hydroxy-4-methyl-phenoxy)-phenyl]-1H-[1,2,3]triazole-4-carboxylic acid;
57. 2-(2-Hydrazino-phenoxy)-5-methyl-phenol;
58. 2-(4-Hydrazino-phenoxy)-5-methyl-phenol;
59. 1-[2-(2-Hydroxy-4-methyl-phenoxy)-phenyl]-1H-pyrazole-4-carboxylic acid ethyl ester;
60. 1-[4-(2-Hydroxy-4-methyl-phenoxy)-phenyl]-1H-pyrazole-4-carboxylic acid ethyl ester;
61. 1-[2-(2-Hydroxy-4-methyl-phenoxy)-phenyl]-1H-pyrazole-4-carboxylic acid;
62. 1-[4-(2-Hydroxy-4-methyl-phenoxy)-phenyl]-1H-pyrazole-4-carboxylic acid;
63. 5-Methyl-2-(2-[1,2,4]triazol-1-yl-phenoxy)-phenol; and
64. 5-Methyl-2-(4-[1,2,4]triazol-1-yl-phenoxy)-phenol.

Descriptions of the compounds of the present invention rely upon terms that include the following:

The compounds of the invention are named according to the IUPAC or CAS nomenclature system. The carbon atoms content of various hydrocarbon-containing moieties is indicated by a prefix designating the minimum and maximum number of carbon atoms in the moiety, i.e., the prefix Ci-Cj indicates a moiety of the integer “i” to the integer “j” carbon atoms, inclusive. Thus, for example, C1-C4 alkyl and cycloalkyl refers to alkyls and cycloalkyls of one to four carbon atoms, inclusive, or methyl, ethyl, propyl, isopropyl, cyclopropyl, butyl and its isomeric forms, and cyclobutyl, cyclopropylmethyl and methylcyclopropyl.

Hydroxylprotecting groups are benzyl, 4-methoxybenzyl, methyl, benzyl, 2,2,2-trichloroethyl, t-butyldimethylsilyl, trimethylsilyl, t-butyl, allyl, or as described in Greene, Theodora W., Protective Groups in Organic Synthesis, 1999, John Wiley & Sons Inc.: Chapter 3.

Unless otherwise specified, the terms “heterocycle”, “heterocyclic group”, or “heterocyclic” are used interchangeably herein and include monocyclic, bicyclic ring or bridged ring systems having from 4-10 atoms, 1-4 of which are selected from O, S and N. The term “heterocyclic group” includes non-aromatic groups such as thiazolidinyl and [1,3]thiazinanyl, and heteroaryl groups such as thiophenyl and oxadiazolyl. The term “aryl” in “heteroaryl” refers to aromaticity, a term known to those skilled in the art and defined in greater detail in “Advanced Organic Chemistry”, M. B. Smith and J. March, 5^{th} Ed., John Wiley & Sons, New York, N.Y. (2001). Preferred heterocyclic groups represented by the term are R, wherein the waved line indicates the bond of attachment. For example, a bond pointing inside a ring such as Zq- in (f)-(iii) indicates that the substitution can connect to any carbon or nitrogen position that is capable of accepting a covalent bond. Heterocyclic groups in the compounds of the invention may be C-attached or N-attached where such is possible.

As is apparent to those of ordinary skill in the art, the compounds of the present invention can exist in tautomeric forms, and all such tautomeric forms are included within the scope of the present invention. For instance, in the compounds of Example 17, the 1H-tetrazolyl group can also exist as the 2H-tetrazol-5-yl group and both such tautomers are included within the scope of the present invention. Geometric isomers of olefins, C═N double bonds, and the like can also be present in the compounds described herein, and all such stable isomers are contemplated in the present invention.

It will be apparent to one skilled in the art that selected heterocyclic ring systems may have chiral centers as well, giving enantiomers and diastereomers. These diastereomers and enantiomers, in racemic, diastereomerically or enantiomerically enriched forms, are also within the scope of the compounds of the invention.

The compounds of the invention are capable of forming both pharmaceutically acceptable acid addition and/or base salts. Base salts are formed with metals or amines, such as alkali and alkaline earth metals or organic amines. Examples of metals used are sodium, potassium, magnesium, calcium, and the like. Examples of suitable amines are N,N′-dibenzylethyldiamine, chloroprocaine, choline, diethanolamine, ethylenediamine, N-methylglucamine, and procaine.

Pharmaceutically acceptable acid addition salts are formed with organic or inorganic acids. Examples of suitable acids for salt formation are hydrochloric, sulfuric, phosphoric, acetic, citric, oxalic, malonic, salicyclic, malic, gluconic, fumaric, succinic, ascorbic, maleic, methanesulfonic, and the like. The salts are prepared by contacting the free base form with a sufficient amount of the desired acid to produce either mono or di, etc. salt in the conventional manner. The free base forms may be regenerated by treating the salt form with a base. For example, dilute solutions of aqueous base may be utilized. Dilute aqueous sodium hydroxide, potassium carbonate, ammonia, and sodium bicarbonate solutions are suitable for this purpose. The free base forms differ from their respective salt forms somewhat in certain physical properties such as solubility in polar solvents, but the salts are otherwise equivalent to their respective free base forms for purposes of the invention.

The compounds of the invention are capable of forming pharmaceutically acceptable prodrugs. “Prodrugs” are considered to be any covalently bonded carriers which release the active parent drug in vivo when such prodrug is administered to a subject. Prodrugs of a compound are prepared by modifying functional groups present in the compounds in such a way that the bonds are cleaved, either in routine manipulation or in vivo, to the parent compounds. Prodrugs include, but are not limited to, compounds wherein hydroxyl, amine, or sulfhydroxyl groups are bonded to any group that, when administered to a subject, cleave to form a free hydroxyl, amino, or sulfhydroxyl group, respectively. Examples of prodrugs include, but are not limited to, acetate, formate, benzoate and phosphate ester derivatives of hydroxyl functional groups, especially the hydroxyl group on A-ring of Formulas I and II, and acetyl and benzoyl derivatives of amine functional groups in the compounds of the invention and the like.

The compounds of the invention can exist in unsolvated as well as solvated forms, including hydrated forms. In general, the solvated forms, including hydrated forms and the like are equivalent to the unsolvated forms for purposes of the invention.

The compounds are of course given in a form suitable for each administration route. For example, they are administered in drops, tablets or capsule form, by injection, inhalation, eye lotion, ointment, foams, suppository, etc. by topical, vaginal or rectal administration. Oral, parenteral or topical administration is preferred. The compounds of the invention are useful for the treatment of infections in hosts, especially mammals, including humans, in particular in humans and domesticated animals. The compounds may be used, for example, for the treatment of infections of skin, mouth, the respiratory tract, the urinary/reproductive tract, and soft tissues and blood, especially in humans. In one embodiment of the invention diseases are those caused by or associated with infection by microorganisms including, but are not limited to, Streptococcus pyogenes, Staphylococcus aureus, methicillin resistant Staphylococcus aureus (“MRSA”), Staphylococcus epidermidis, Bacillus anthracis, Neisseria gonorrhoeae, Neisseria meningitidis, Mycobacteria tuberculosis, vancomycin resistant Enterococcae (“VRE”), Helicobacter pylori, Chlamydia pneumoniae, Chlamydia trachomatis, Campylobacter jejuni, Propionibacterium acnes, Pseudomonas aeruginosa, Haemophilus influenzae, Streptococcus pneumoniae, Enterococcus faecalis, Haemophilus influenzae, Escherichia coli, Corynebacterium diphtheriae, Moraxella catarrhalis and Bacillus cereus.

The pharmaceutical compositions of the present invention employ the compounds of the invention and may include inert, pharmaceutically acceptable carriers that are either solid or liquid. Solid form compositions include powders, tablets, dispersible granules, capsules, cachets and suppositories. A solid carrier can be one or more substances which may also act as diluents, flavoring agents, solubilizers, lubricants, suspending agents, binders, or tablet disintegrating agents; it can also be an encapsulating material. In powders, the carrier is a finely divided solid which is an admixture with the finely divided active compound. In the tablet the active compound is mixed with carrier having the necessary binding properties in suitable proportions and compacted in the shape and size desired. The powder and tablet preferably contain from 5 to about 70 percent, and preferably from 10 to about 60 percent of the active ingredient. Suitable solid carriers are magnesium carbonate, magnesium stearate, talc, sugar, lactose, pectin, dextrin, starch, gelatin, tragacanth, methyl cellulose, sodium carboxymethyl cellulose, a low melting wax, cocoa butter, and the like.

The term “preparation” is intended to include compositions wherein the formulation of the active compound with encapsulating material acts as carrier. This provides a capsule in which the active component (with or without other carriers) is surrounded by a carrier, which is accordingly in association with it. Similarly, cachets are included. Tablets, powders, cachets, and capsules can be used as solid dosage forms suitable for oral administration.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in formulations, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils, glycerol, polyethylene glycols and fatty acid esters of sorbitan, and mixtures thereof. Aqueous solutions suitable for oral use can be prepared by dissolving the active component in water and adding suitable colorants, flavors, stabilizing, and thickening agents as desired. Aqueous suspensions suitable for oral use can be made by dispersing the finely divided active component in water with viscous material, i.e. natural or synthetic gums, resins, methyl cellulose, sodium carboxymethyl cellulose, and other well-known suspending agents.

An example, for instance, is the use of water or water-propylene glycol solutions for parenteral injection. Such solutions are prepared so as to be acceptable to biological systems (isotonicity, pH, etc). Liquid preparations can also be formulated in solution in aqueous polyethylene glycol solution.

Formulations of the present invention which are suitable for topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery over time of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

The compositions of the invention may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

A physician or veterinarian having ordinary skill in the art can readily determine and prescribe the effective amount of the pharmaceutical composition required. For example, the physician or veterinarian could start doses of the compositions of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, topical, intravenous and subcutaneous doses of the compositions of this invention for a patient, when used for the indicated effects, will range from about 0.0001 to about 100 mg per kilogram of body weight per day, more preferably from about 0.01 to about 50 mg per kg per day, and still more preferably from about 0.1 to about 10 mg per kg per day. Each unit dose may be, for example, 5, 10, 25, 50, 100, 125, 150, 200 or 250 mg of the compound of the invention. If desired, the effective daily dose of the active compound may be administered as two, three, four, five, six or more sub-doses administered separately at appropriate intervals throughout the day, optionally, in unit dosage forms.

Synthesis

The compounds of the present invention may be synthesized according to the chemistry outlined in the following schemes. It will be apparent to those skilled in the art that the described synthetic procedures are merely representative in nature and that alternative procedures are feasible and may be preferred in some cases.

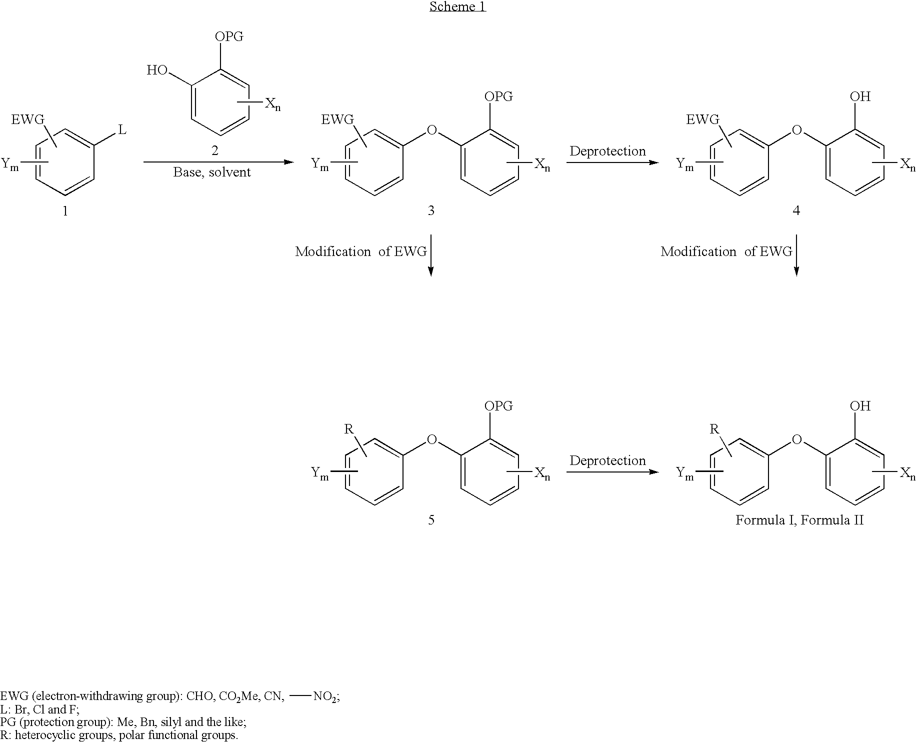

The chemistry to synthesize diaryl ether derivatives is well known to those skilled in the art of organic chemistry and has been well documented recently by 1) Theil, F, Angew. Chem. Int. Ed. 1999, 38, 2345; 2) Sawyer, J. S., Tetrahydron, 2000, 56, 5045; 3) Ley, S. V. et al, Angew. Chem. Int. Ed. 2003, 42, 5400. Many of the strategies and reactions can be applied to the synthesis of 2-aryloxyphenol derivatives in the invention of Formula I and Formula II.

Key intermediates 3 may be synthesized directly by the coupling reaction of mono-protected catechol derivatives 2 with aryl halides 1 bearing electron withdrawing groups (EWG) such as CN, CHO, NO₂, CO₂Me etc. at para- or ortho-position through S_{N}Ar mechanism as described in Scheme 1. The first step may be attained by heating the reaction media at 30-150° C. optionally with the assistance of ultrasonic or microwave irradiation, with an excess amount of a base in a solvent. Excess refers to any amount of base exceeding one equivalent. Bases which may be used in the reactions include irreversible bases such as NaH, KH, LiH, lithium diisopropylamide (LDA) etc; reversible bases such as sodium tert-butoxide, potassium tert-butoxide and the like; equilibrating bases such as Na₂CO₃, K₂CO₃, Cs₂CO₃, CsF, MgO, CaO, trialkylamines, pyridine, 4-(dimethylamino)pyridine (DMAP), N-methylmorphorine, diethyl-1-propylamine and the like. Solvents suitable for the reactions include ethereal or non-protic solvents such as N,N-dimethylacetamide (DMA), N,N-dimethylformamide (DMF), acetonitrile (ACN) and the like.

Deprotection of 3 gives another intermediate 4. Most commercial or literature mono-protected catecols 2 bear methyl, benzyl, or silyl groups as hydroxylprotection groups. Such protection groups may be removed from products with acids, contact hydrogenation, BBr₃, BI₃, MgI₂, NaSEt, tetrabutylammonium fluoride etc in protic or aprotic solvents at −78° C.-100° C. Intermediate 4 may be converted to the compounds of this invention by converting EWG (electron withdrawing groups) groups into heterocyclic groups or highly polar functional groups. The compounds can also be obtained by initially converting 3 to 5 by the modification of the EWG groups, followed by the deprotection step.

The introduction of heterocyclic group R by converting various EWG groups in intermediates 3 and 4 is demonstrated in Scheme 2-4.

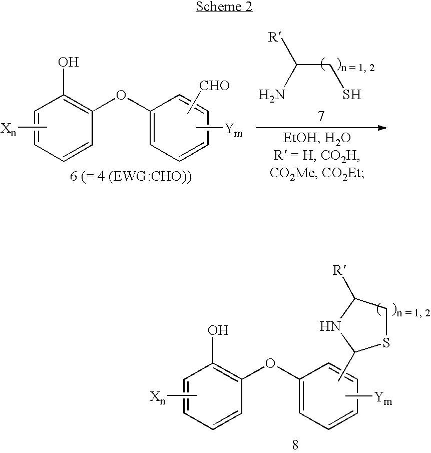

For example, condensation of aldehyde 6 (=4 (EWG: CHO)) with β or γ-hydroxy- or mecapto-alkylamines 7 in aqueous alcohol affords thiazolidine and thiazinane derivatives 8 (Formula I and II, wherein R is moiety (s)). (Scheme 2)

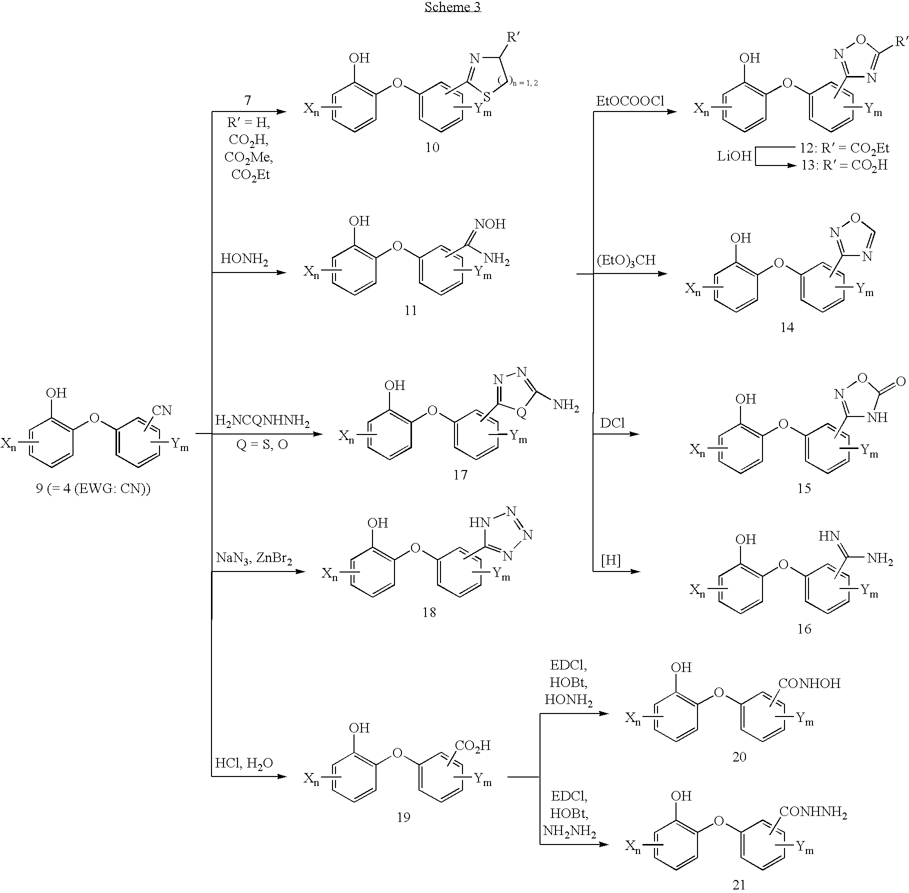

Alternatively, compounds 9 (=4 (EWG: CN)) may be reacted with 7 in aqueous alcohol to give heterocyclic compounds 10 (Formula I and II, wherein R is (t) (Scheme 3). By reacting with hydroxylamine, compound 9 can conveniently be converted into N-hydroxyl-amidines 11 (Formula I and II, wherein R is C(NOH)NH₂) which may further be cyclized to 1,2,4-oxadiazoles 12, 14 and 15 (Formula I and II, wherein R is (O)-(i) and (r)) by the reactions with ethyl oxalyl chloride, triethyl orthoformate and carbonyl diimidazole respectively. Compound 12 can be hydrolyzed to acid 13, which may be easily converted to other acid derivatives. Contact hydrogenation of 11 provides amidine 16 (Formula I and II, wherein R is C(NH)NH₂). Reactions of compound 9 with semicarbazide and thiosemicarbazide in organic acids, typically trifluoroacetic acid (TFA) give rise to 1,3,4-oxadiazoles and thiodiazoles 17 (Formula I and II, wherein R is (m) and (n)) respectively. Tetrazole derivatives 18 (Formula I and II, wherein R is (p)-(i) and (ii)) may be attained by the reaction of 9 with sodium azide and zinc bromide in aqueous protic or ethereal solvent such as isopropanol and dioxane. The reaction temperature is, typically 50-120° C. Compound 9 can also be hydrolyzed into corresponding acid 19 under standard conditions well known in the art. Acid 19, in turn, may be converted to various acid derivatives by reactions well known in the art. For example, the active ester of 19 formed by the reaction with 1-ethyl-3-(3-dimethylaminopropyl)-carbodiimide (EDCI) and 1-hydroxybenzotriazole (HOBt) may react with hydroxyamine and hydrazine to give N-hydroxy amide 20 and carbazide 21 respectively (Formula I and II, wherein R is C(O)NHOH and C(O)NHNH₂).

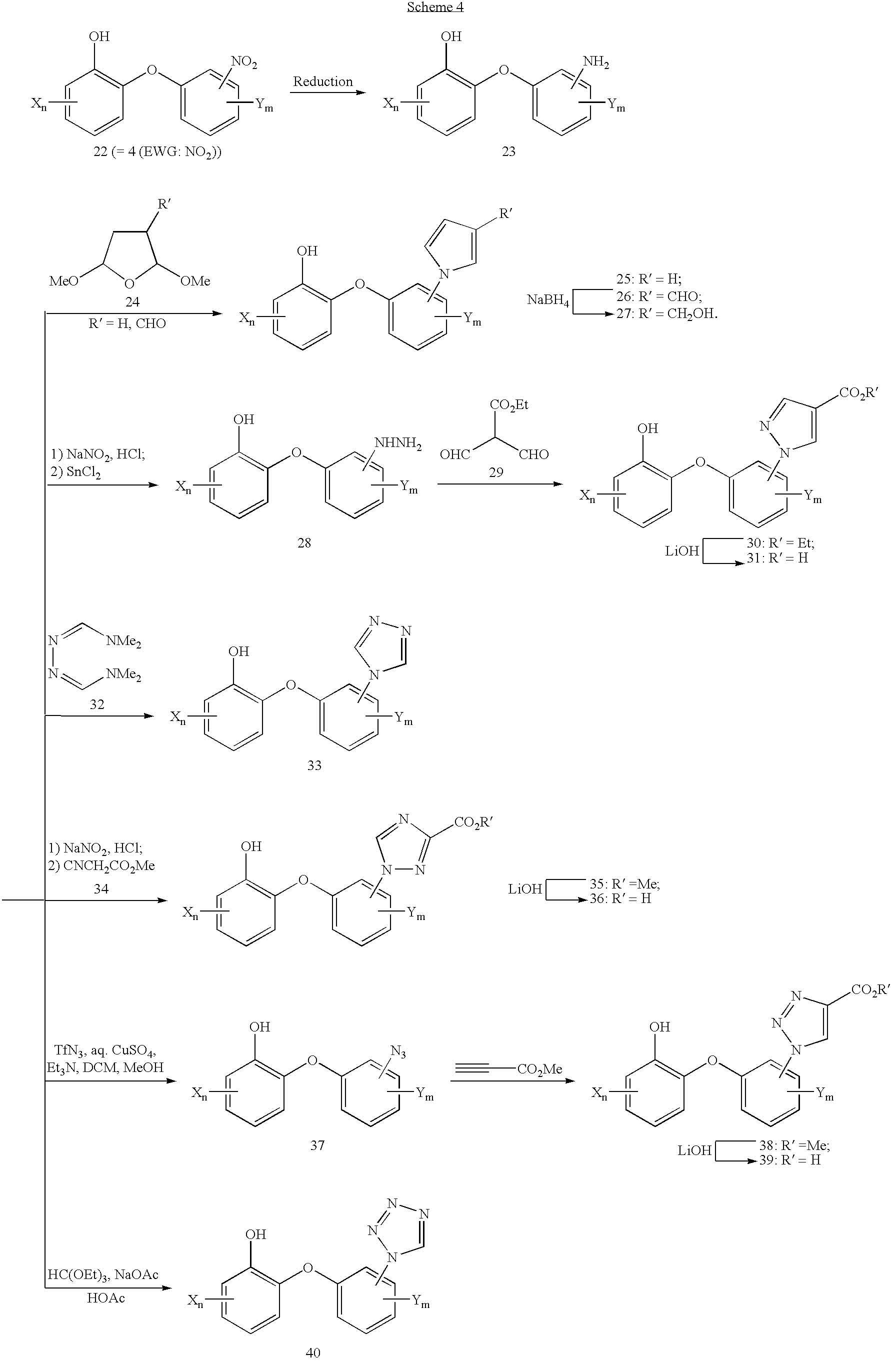

Similarly, nitro compound 22 (=4 (EWG: NO₂)) may be converted to the compounds of present invention as well through amine 23 by standard reactions well known in the art of organic synthesis. Reduction of 22 to 23 (Scheme 4, Equ. 1) may be achieved in light of standard contact hydrogenation in the assistance of palladium on charcoal in protic solvents, typically methanol in hydrogen atmosphere. Alternatively, amine 23 can be obtained by the reduction of 22 with tin (II) chloride in ethanol and DMF at the temperature, typically, of 20-100° C.

Condensation of 23 with reagents 24 affords pyrrole derivatives 25 or 26 (Formula I and II, wherein R is (c)-(ii)). Further reduction of 26 with sodium borohydride yields alcohol 27. (Scheme 4, Equ. 2) Diazotization of 23 with sodium nitrite and hydrochloric acid, followed by reduction with tin (II) chloride provides hydrazine 28 (Formula I and II, wherein R is NHNH₂), which then reacts with dialdehyde 29 and sodium acetate in ethanol to give pyrazole derivative 30 (Formula I and II, wherein R is (f)-(iii)). Alternatively, 23 may be converted to 4H-1,2,4-triazole derivative 33 (formula I and II, wherein R is (k)-(ii)) conveniently by its reaction with reagent 32 in a solvent such as N,N-dimethylacetamide (DMA), N,N-dimethylformamide (DMF) and the like at 30-120° C. Diazotization of 23, followed by 1,3-dipolar cycloaddition with methyl isocyanoacetate 34 affords 1H-1,2,4-triazole derivative 35 (Formula I and II, wherein R is (1)-(ii)) The cyclization process is typically run in aqueous NaHCO₃ media at 0-30° C. Compound 23 may also be converted to azide 37 by a reaction originally reported by Liu (Liu, Q. et al, Org. Lett., 2003, 5, 2571), using freshly prepared triflyl azide in aqueous CuSO₄, dichloromethane (DCM) and methanol in the presence of triethylamine at 0-50° C. Subsequently, 37 may undergo 1, 3-dipolar cycloaddition with methyl propionate in toluene or benzene at reflux, giving 1H-1,2,3-triazole derivative 38 (Formula I and II, wherein R is (j)-(iii)). Furthermore, reaction of 23 with triethyl orthoformate and sodium acetate in acetic acid at the temperature, typically 60-120° C. affords 40 (Formula I and II, wherein R is (q)) a tetrazolyl derivative with bond attachment on nitrogen. Corresponding acids and their derivative of heterocyclic esters 30, 35 and 38 may be attained by following hydrolysis procedures well known in the art of organic synthesis.

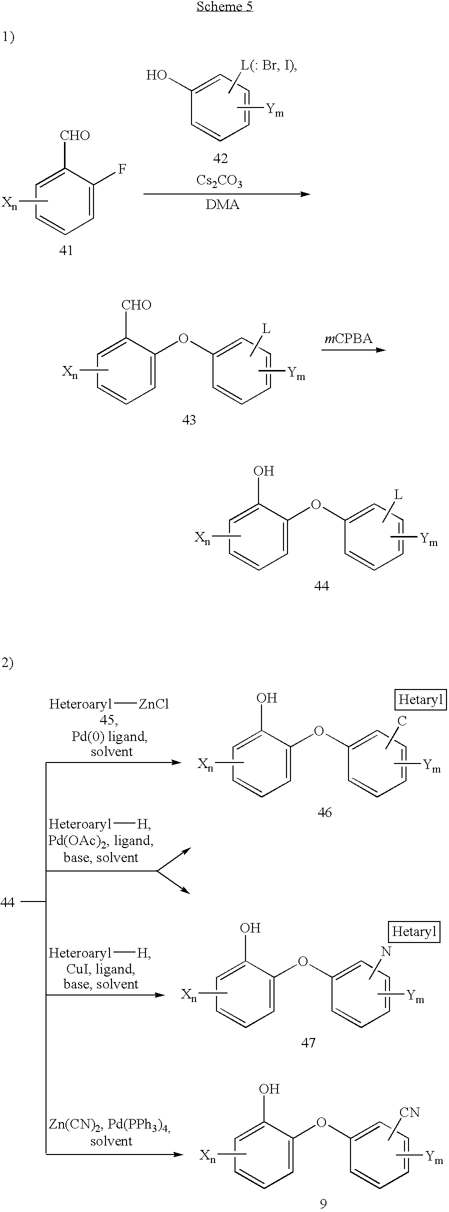

The heterocyclic groups R may be introduced into the precursors 44 to form the compounds of present invention of Formula I and II by the coupling reactions with heterocyclic reagents, promoted by transition-metal catalysts. (Scheme 5) Compounds 44 may be synthesized by condensation of 2-fluoro-benzaldehydes 41 with halo-phenols 42 in the assistance of cesium carbonate, followed by a Baeyer-Villiger oxidation with mCPBA (Scheme 5, Equ. 1). As described in Scheme 5, Equ. 2), bromo or iodo substituted precursors 44 can undergo transition-metal catalyzed cross-coupling reactions with heteroaryl —ZnCl or heteroaryl-H under the conditions well known in the art. For example, a C—C bond formation between 44 and heterocyclic reagents may be accomplished through a coupling reaction promoted by limited quality of Pd(PPh₃)₄ or Pd(OAc)₂ along with limited quality of a ligand in the presence of excess of base in a solvent, giving Formula I and II compounds 46. Alternatively, a C—N bond formation may be favored when the reaction is performed with the assistance of limited quality of Pd(OAc)₂ or CuI with limited quality of a ligand in the presence of excess base in a solvent, giving compounds 47. Similarly, the reaction of 44 with excess of Zn(CN)₂ in the presence of limited quality of Pd(PPh₃)₄ in DMF provides corresponding cyano compounds 9 which may be, in turn, converted to other Formula I and II compounds by following the synthetic routes described in Scheme 3.

Heterocyclic reagents such as heteroaryl-H in Scheme 5, are in the literature or commercially available. Heteroaryl-ZnCl is well known in the art and may be prepared in situ from corresponding heteroaryl-halides by following the literature, for example, J. Med. Chem., 2003, 46, 265. The limited quality refers to 1.0 mol %-20 mol % relative to substrate. Bases suitable for the reaction includes equilibrating bases such as Na₂CO₃, K₂CO₃, Cs₂CO₃, K₃PO₄, Na₃PO₄, MgO, CaO and reversible bases such as NaO^{t}Bu and KO^{t}Bu. The ligands used in the reaction are, but not limited to PPh₃, 2,2′-bis(diphenylphosphino)-1,1′-binaphtyl (BINAP), 1,1′-bis(diphenylphosphino)ferrocene (DPPf), P(o-tolyl)₃, P(t-Bu)₃, biphenyl-2-yl-di(tert-butyl)-phosphane, di(tert-butyl)-(2′-methyl-biphenyl-2-yl)-phosphane, [2′-(di(tert-butyl)-phosphanyl)-biphenyl-2-yl]-dimethyl-amine, trans-1,2-cyclohexanediamine and the like. Solvents which may be used in the reactions include THF, 1,4-dioxane, toluene, DMA, DMF and the like. The cross-coupling reaction proceeds at −78° C.-150° C.

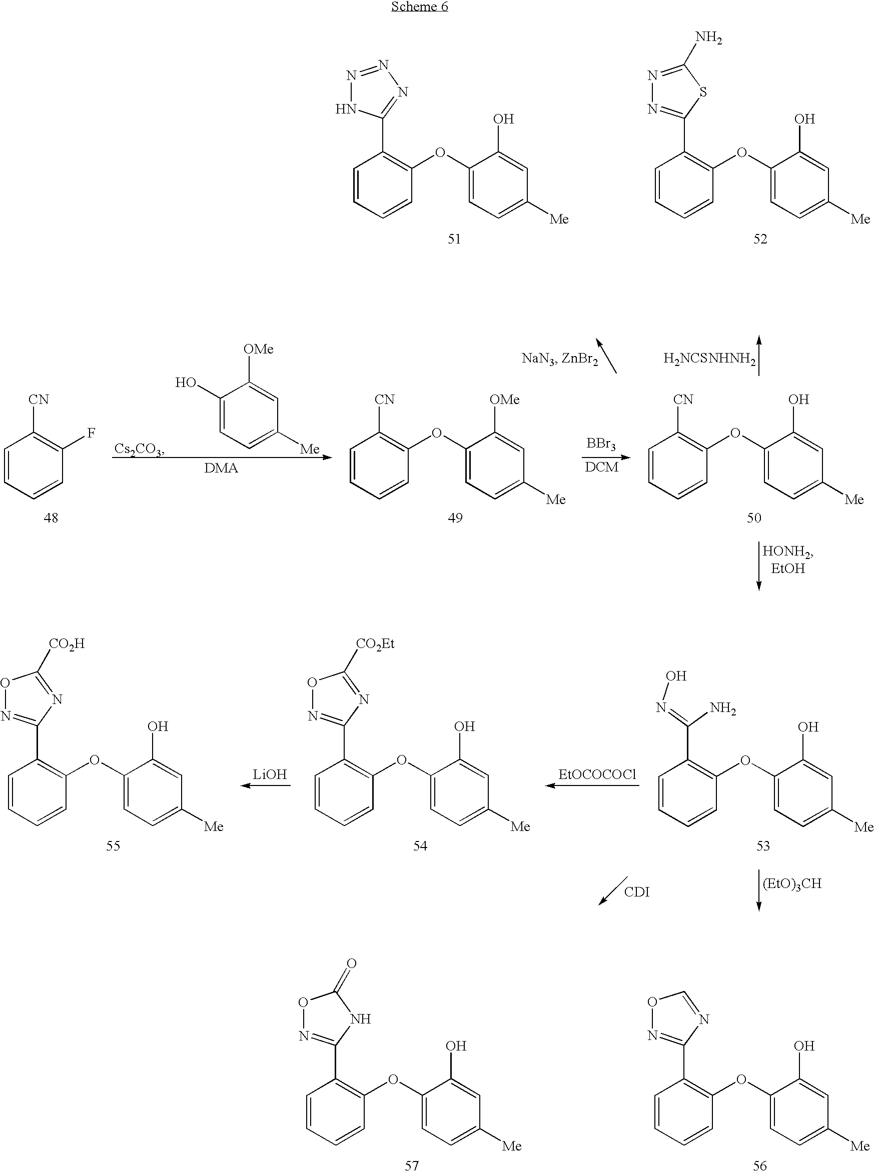

Scheme 6 demonstrates the syntheses of specific compounds depicted by Formula I of present invention. Reaction of 2-fluoro-benzonitrile 48 with 2-methoxy-4-methyl-phenol in the presence of cesium carbonate in DMA gave 49, which was demethylated to 2-hydroxy-aryloxyphenol 50 by its reaction with boron tribromide in dichloromethane (DCM). Using known procedure described by Sharpless et al, Org. Lett., (2002) 4, 2525, compound 50 was transformed to tetrazole 51 by the reaction with sodium azide and zinc bromide. Alternatively, compound 50 was conveniently converted to thiodiazole 52 by the reaction with thiosemicarbazide in trifluoroacetic acid. Reaction of 50 with hydroxylamine in aqueous ethanol afforded N-hydroxylamidine 53. Cyclization of 53 employing ethyl oxalyl chloride, triethyl orthoformate and carbonyl diimidazole gave oxadiazoles 54, 56 and 57 respectively. Hydrolysis of 54 using lithium hydroxide provided corresponding acid 55. The synthetic sequence in Scheme 6 was applicable to synthesize other compounds depicted by general Formula I when substituted 2-fluoro-benzonitriles and substituted 2-methoxyphenols were employed as starting materials. Corresponding Formula II compounds of the present invention, i.e. the region isomers of the compounds described in Scheme 6 were synthesized by employing 4-fluoro-benzonitrile and its derivatives as starting materials.

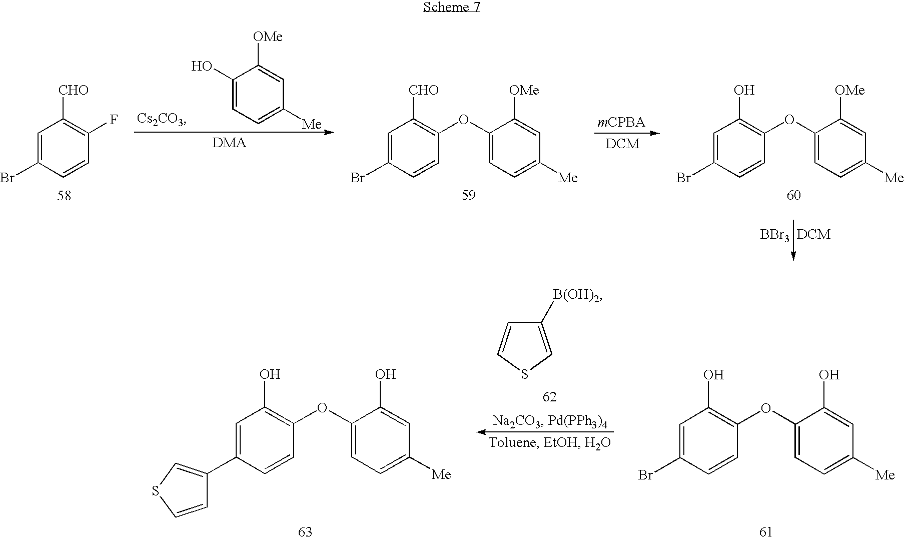

Scheme 7 shows a synthetic route to specific compounds depicted by Formula II via a heterocyclic cross-coupling reaction outlined in Scheme 5. Reaction of benzaldehyde 58 with 2-methoxy-4-methyl-phenol in the presence of cesium carbonate provided 59, which was converted to compounds 60 via Baeyer-Villiger oxidation. Reaction of 60 employing boron tribromide as deprotective agent gave bromo-bisphenol 61. Suzuki coupling of 61 with 3-thiophenylboronic acid 62 in the presence of palladium (0) catalyst and sodium carbonate afforded Formula II compound 63. Similar reactions of compound 61 with other commercially or literature available heteroaryl boronic acids provided many compounds covered by Formula II.

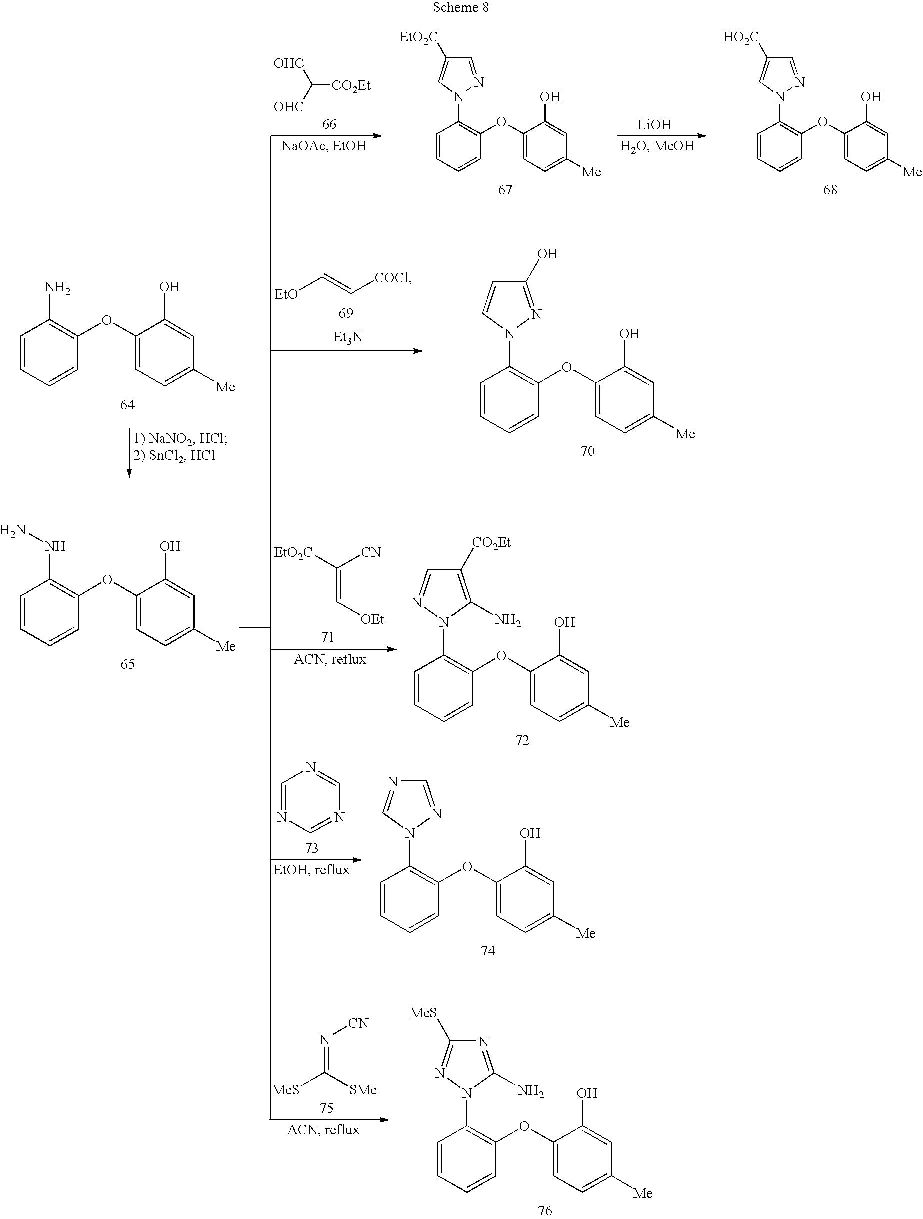

Scheme 8 shows the synthesis of specific compounds of Formula I of the present invention by following the methodology outlined in Scheme 4. Thus, diazotization of 64 with sodium nitrite followed by the reduction with of tin (II) chloride afforded hydrazine 65. Cyclization of 65 with dialdehyde 66, prepared by the procedure described by Bertz et al (J. Org. Chem., 1982, 47, 2216), provided pyrazole 67, which was hydrolyzed to corresponding acid 68. Cyclization of 65 with acid chloride 69, made by following a literature procedure described by Tietze et al (Synthesis, 1993, 1079), in the presence of triethylamine gave pyrazole 70. Alternatively, reaction of 65 with 2-cyano-3-methoxy-acrylic acid ethyl ester 71 in acetonitrile, as disclosed by Hwang et al (J. Comb. Chem., 2005, 7, 136), led to amino-pyrazole 72. [1,2,4]-Triazoles 74 and 76 were accomplished from 65 by the cyclization with 1,3,5-triazine 73, as disclosed by Grundmann et al (J. Org. Chem., 1956,21,1037) and dimethylcyanothioimnocarbonate 75 respectively. The Formula II isomers of 67, 68, 72, 74 and 76 were attained by the reactions disclosed in Scheme 8 with 2-(4-amino-phenoxy)-5-methyl-phenol 77 instead of 64.

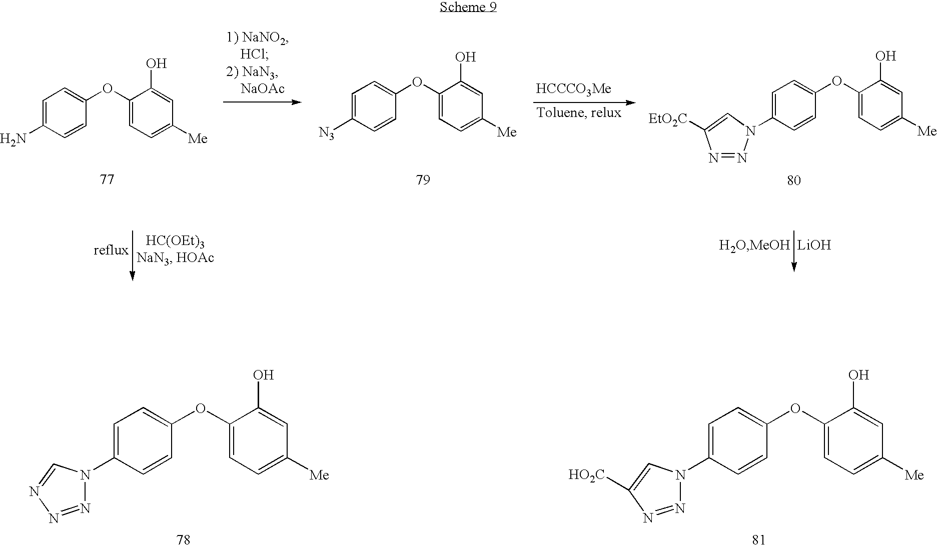

Scheme 9 shows the synthesis of specific compounds of Formula II of the present invention by following the methodology outlined in Scheme 4. Reaction of amine 77 with triethyl orthoformate and sodium acetate in refluxed acedic acid afforded tetrazole 78. Alternatively, diazotization of 77, followed by the substitution with sodium azide provided azide 79 in good yield. 1,3-dipolar cycloaddition of 79 with methyl propiolate under the conditions disclosed by Genin et al (J. Med. Chem., 2000, 43, 953) gave [1,2,3]triazole 80, which was hydrolyzed to acid 81. The Formula I isomers of 78, 79, 80 and 81 were attained by the reactions disclosed in Scheme 9 with 2-(2-amino-phenoxy)-5-methyl-phenol 64 instead of 77.

### EXAMPLES

The following specific examples are provided for the purpose of further illustration only and are not intended to limit the disclosed invention.

### Example 1

### 2-(2-Hydroxy-phenoxy)-5-(thiophen-3-yl)-phenol

### Step 1: 2-(4-Bromo-2-methoxy-phenoxy)-benzaldehyde

A suspension of 2-fluoro-benzaldehyde (1.223 g, 9.850 mmol), 4-bromo-2-methoxy-phenol (2.000 g, 9.850 mmol) and cesium carbonate (3.530 g, 10.84 mmol) in N,N-dimethylacetamide (DMA) (20 mL) was stirred at 100° C. for 4 hrs. The reaction mixture was poured into water and extracted with ether (30 mL×2). The organic layer was washed with water (30 mL), dried over anhydrous sodium sulfate, filtered and evaporated. The crude solid residue was triturated in hexane and dried in vacuum. The final product was obtained as white powder (2.520 g, 83%). (melting point) M.P.: 81-83° C.

### Step 2: 2-(4-Bromo-2-methoxy-phenoxy)-phenol

To a suspension of 2-(4-bromo-2-methoxy-phenoxy)-benzaldehyde (2.300 g, 7.49 mml), NaH₂PO₄ (2.696 g, 22.47 mmol) in dichloromethane (DCM) (20 mL) was added in portions of m-chloro-perbenzoic acid (1.679 g, 7.490 mmol). After being stirred at ambient temperature for 4 hrs, the reaction mixture was quenched with saturated aqueous sodium sulfite solution (10 mL) and stirred for another 30 min. The organic layer was separated and the aqueous layer was extracted with dichloromethane (DCM) (20 mL×2). The combined organic phase was washed with saturated sodium carbonate solution, dried over anhydrous sodium sulfate, filtered and evaporated. The solid residue was washed with hexane and dried in vacuum. The product (2.010 g, 91%) was obtained as white powder, which was used for next reaction without further purification. M.P.: 107-109° C.

### Step 3: 5-Bromo-2-(2-methoxyphenoxy)-anisole

To a suspension of 2-(4-bromo-2-methoxy-phenoxy)-phenol (2.500 g, 7.50 mmol) and Cs₂CO₃ (3.665 g, 11.25 mmol) in acetonitrile was added dropwise methyl iodide (0.93 mL, 15.00 mmol) via syringe. After being stirred at room temperature for 2 days, the mixture was poured into water and extracted with DCM (30 mL×2). The combined layer was washed with water, dried over sodium sulfate, filtered and evaporated. The residue was purified by flash chromatography on silica column eluted with 15% of ethyl acetate (AcOEt) in hexane. The final product (1.925 g, 83%) is a white solid. M.P.: 58-60° C.

### Step 4: 3-[3-Methoxy-4-(2-methoxy-phenoxy)-phenyl]-thiophene

A pressure tube was charged with a suspension of 5-bromo-2-(2-methoxyphenoxy)-anisole (200 mg, 0.647 mmol), 3-thiophene-boronic acid (99.4 mg, 0.78 mmol), sodium carbonate (165 mg, 1.553 mmol), Pd(PPh₃)₄ (30 mg), toluene (6 mL), EtOH (2 mL) and water (2 mL). After being heated and stirred vigorously at 110° C. for 3 days, the reaction mixture was poured into water, extracted with ether (20 mL×3). The ether layer was washed with water, dried over anhydrous sodium sulfate, filtered and evaporated. The crude residue was purified by column chromatography on silica gel, eluted with 30% of ethyl acetate in hexane, giving the final product as oil. C₁₈H₁₆O₃S (312.08): gas chromatography mass spectrometry (GC-MS) (electron impact (EI)+) m/e: 312.

### Step 5: 2-(2-Hydroxy-phenoxy)-5-(thiophen-3-yl)-phenol

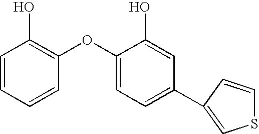

To a solution of 3-[3-methoxy-4-(2-methoxy-phenoxy)-phenyl]-thiophene (150 mg, 0.480 mmol) in DCM (2 mL) on an acetone-dry ice bath was added dropwise BBr₃ (0.091 mL) via syringe. The cooling bath was removed and the reaction mixture was stirred at room temperature for 5 hrs. The mixture was quenched with methanol (1 mL) and subsequently water (2 mL), and extracted with ethyl acetate (2 ml×3). The organic layer was washed with saturated sodium chloride solution and dried on anhydrous sodium sulfate. The residue was chromatographed over a silica gel column, eluting with 30% of ethyl acetate (AcOEt) in hexane to give the Example title compound [2-(2-Hydroxy-phenoxy)-5-(thiophen-3-yl)-phenol] (120 mg, 88%) as white powder. M.P.: 114-116° C.; C₁₆H₁₂O₃S (284.0507): high resolution mass spectrometry (HRMS) (EI+) m/e: 284.0499. This product was also analyzed by proton nuclear magnetic resonance (¹H-NMR). The corresponding ¹H-NMR spectrum was consistent with the structure of anticipated product.

### Example 2

### 2-(2-Hydroxy-4-methyl-phenoxy)-5-(thiophen-3-yl)-phenol

### Step 1: 5-Bromo-2-(2-methoxy-4-methyl-phenoxy)-benzaldehyde

A suspension of 5-bromo-2-fluoro-benzaldehyde (1.470 g, 7.240 mmol), 2-methoxy-4-methyl-phenol (1.000 g, 7.240 mmol) and cesium carbonate (2.830 g, 8.690 mmol) in DMA (10 mL) was stirred at 100° C. overnight. The reaction mixture was poured into water (20 mL) and extracted with ether (30 mL×3). The organic layer was washed with water (30 mL), dried over anhydrous sodium sulfate, filtered and evaporated. The solid residue was triturated in hexane and dried in vacuum, giving the product (1.270 g, 55%) as white powder. M.P.: 89-90° C.; C₁₅H₁₃BrO₃ (320.00): GC-MS (EI+) m/e: 320.

### Step 2: 5-Bromo-2-(2-methoxy-4-methyl-phenoxy)-phenol

To a suspension of 2-(4-bromo-2-methoxy-phenoxy)-benzaldehyde (1.200 g, 7.49 mml), NaH₂PO₄ (2.692 g, 22.44 mmol) in DCM (20 mL) was added 70% m-chloro-perbenzoic acid (2.008 g, 8.960 mmol) in portions. After being stirred at room temperature for 2 days, the reaction mixture was quenched with saturated aqueous sodium sulfite solution (20 mL) and stirred for another 30 min. The organic layer was separated and the aqueous layer was extracted with DCM (20 mL×2). The combined organic phase was washed with saturated sodium carbonate solution, dried over anhydrous sodium sulfate, filtered and evaporated. The residue was chromatographed on silica column, eluting with 15% of ethyl acetate in hexane. The product (546 mg, 47%) was obtained as white powder. M.P.: 75-76° C.

### Step 3: 5-Bromo-2-(2-hydroxy-4-methyl-phenoxy)-phenol

To a solution of 5-bromo-2-(2-methoxy-4-methyl-phenoxy)-phenol (500 mg, 1.62 mmol) in DCM (5 mL) cooled on an acetone-dry ice bath was added dropwise BBr₃ (0.153 mL, 1.620 mmol) via syringe. The cooling bath was removed and the reaction mixture was stirred at ambient temperature for 5 hrs. The mixture was quenched with methanol (1 mL), and subsequently brine (5 mL) and extracted with DCM (5 ml×3). The combined organic layer was washed with saturated sodium chloride solution, dried on anhydrous sodium sulfate, filtered and evaporated. The solid residue was further washed with hexane and dried in vacuum to give the Example title compound (430 mg, 90%) as off-white powder. M.P.: 95-97° C.; C₁₃H₁₁BrO₃ (293.99): GC-MS (EI+) m/e: 294.

### Step 4: 2-(2-Hydroxy-4-methyl-phenoxy)-5-(thiophen-3-yl)-phenol

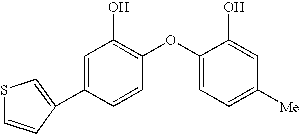

A pressure tube was charged with a mixture of 5-bromo-2-(2-hydroxy-4-methyl-phenoxy)-phenol (100 mg, 0.323 mmol), 3-thiophene-boronic acid (45.54 mg, 0.357 mmol), sodium carbonate (86.24 mg, 0.812 mmol), Pd(PPh₃)₄ (15 mg), toluene (3 mL), EtOH (1 mL) and water (1 mL). After being heated and stirred vigorously at 110° C. for 2 days, the reaction mixture was poured into water and extracted with DCM (20 mL×3). The organic layer was washed with brine, dried over anhydrous sodium sulfate, filtered and evaporated. The crude residue was purified by column chromatography on silica gel, eluted with 30% of ethyl acetate in hexane to provide the product (81 mg, 80%) as white powder. M.P.: 143-145° C.; C₁₇H₁₄O₃S (298.0664): (high resolution mass spectra (electron chemical ionization mode)) HRMS (EI+) m/e: 298.0669. This product was also analyzed by ¹H-NMR. The corresponding ¹H-NMR spectrum was consistent with the structure of the anticipated product.

### Example 3

### 2-(2-Hydroxy-4-methyl-phenoxy)-5-(thiophen-2-yl)-phenol

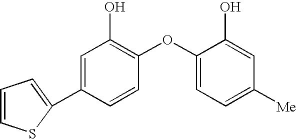

A pressure tube was charged with a mixture of 5-bromo-2-(2-hydroxy-4-methyl-phenoxy)-phenol (100 mg, 0.323 mmol), 2-thiophene-boronic acid (52.1 mg, 0.407 mmol), sodium carbonate (86.24 mg, 0.812 mmol), Pd(PPh₃)₄ (15 mg), toluene (3 mL), EtOH (1 mL) and water (1 mL). After being heated and stirred vigorously at 110° C. for one day, the reaction mixture was poured into water, extracted with DCM (20 mL×3). The organic layer was washed with brine, dried over anhydrous sodium sulfate, filtered and evaporated. The crude residue was purified by column chromatography on silica gel, eluted with 30% of ethyl acetate in hexane to provide the Example title compound (83 mg, 84%) as a white solid. M.P.: 91-92° C.; C₁₇H₁₄O₃S (298.0664): HRMS (EI+) m/e: 298.0659. This product was also analyzed by ¹H-NMR. The corresponding ¹H-NMR spectrum was consistent with the structure of the anticipated product.

### Example 4

### 2-(2-Hydroxy-4-methyl-phenoxy)-5-(furan-2-yl)-phenol

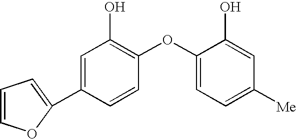

A pressure tube was charged with a mixture of 5-bromo-2-(2-hydroxy-4-methyl-phenoxy)-phenol (100 mg, 0.323 mmol), 2-furan-boronic acid (45.54 mg, 0.357 mmol), sodium carbonate (86.24 mg, 0.812 mmol), Pd(PPh₃)₄ (15 mg), toluene (3 mL), EtOH (1 mL) and water (1 mL). After being heated and stirred vigorously at 110° C. for 3 days, the reaction mixture was poured into water, extracted with DCM (20 mL×3). The organic layer was washed with brine, dried over anhydrous sodium sulfate, filtered and evaporated. The crude residue was purified by column chromatography on silica gel, eluted with 30% of ethyl acetate in hexane to provide two fractions (F-1(top): 41 mg, F-2 (low): 19 mg). Further purification of F1 by chromatography on silica column eluted with 5% ethyl acetate in DCM afforded the final product (31 mg, 32%) as an oil. R_{f} (ethyl acetate:hexane=30%): 0.35; C₁₇H₁₄O₄(282.0892): HRMS (EI+) m/e: 282.0892. This product was also analyzed by ¹H-NMR. The corresponding ¹H-NMR spectrum was consistent with the structure of the anticipated product.

### Example 5

### 5-Methyl-2-[4-(thiazolidin-2-yl)-phenoxy]-phenol

### Step 1: 4-(2-Methoxy-4-methyl-phenoxy)-benzaldehyde

A suspension of 4-fluoro-benzaldehyde (1.000 g, 7.24 mmol), 2-methoxy-4-methyl-phenol (0.898 g, 7.24 mmol) and Cs₂CO₃ (2.830 g, 8.690 mmol) in DMA (10 mL) was stirred and heated at 100° C. overnight. The reaction mixture was quenched with water (20 mL) and extracted with ether (20 mL×3). The organic layer was washed with water and dried on anhydrous sodium sulfate. The residue was chromatographed on silica column, eluting with 15-30% of ethyl acetate in hexane, providing the Example title compound (1.208 g, 69%) as an oil. C₁₅H₁₄O₃ (242.09): GC-MS (EI+) m/e: 242.

### Step 2: 4-(2-Hydroxy-4-methyl-phenoxy)-benzaldehyde

To a solution of 4-(2-methoxy-4-methyl-phenoxy)-benzaldehyde (750 mg, 3.100 mmol) in DCM (5 mL) cooled on an acetone-dry ice bath was added dropwise BBr₃ (0.590 mL, 6.190 mmol) via syringe. The reaction mixture was allowed to warm up to ambient temperature and stirred for 5 hrs. The mixture was quenched with methanol (1 mL), subsequently brine (10 mL), and extracted with DCM (10 ml×3). The combined organic layer was washed with saturated sodium chloride solution, dried on anhydrous sodium sulfate, filtered and evaporated. The residue was chromatographed on silica gel eluting with 30% of ethyl acetate in hexane. The final product (420 mg, 59%) was obtained as white powder. M.P.: 46-48° C.; C₁₄H₁₂O₃ (228.08): GC-MS m/e 228.

### Step 3: 5-Methyl-2-[4-(thiazolidin-2-yl)-phenoxy]-phenol

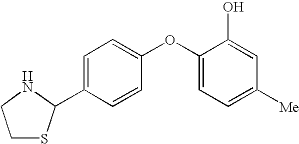

To a stirred suspension of 4-(2-hydroxy-4-methyl-phenoxy)-benzaldehyde (100 mg, 0.438 mmol) and histamine hydrochloride (56 mg, 0.482 mmol) in DCM (2 mL) was added triethylamine (0.067 mL) dropwise. After being stirred at ambient temperature for 5 hrs, the reaction mixture was diluted with DCM (10 mL), washed with water (5 mL×2). The organic layer (suspension) was mixed with methanol (1 mL) (became clear solution), dried on anhydrous sodium sulfate, filtered and evaporated. The solid residue was washed with DCM/hexane (1:1) and dried in vacuum, giving the Example title compound [5-Methyl-2-[4-(thiazolidin-2-yl)-phenoxy]-phenol] (63 mg, 50%) as white powder. M.P.: 197-199° C.; C₁₆H₁₇NO₂S (287.0980): HRMS (EI+) m/e: 287.0974. The product was also analyzed by ¹H-NMR. The corresponding ¹H-NMR spectrum was consistent with the structure of the anticipated product.

### Example 6

### 2-[4-(2-Hydroxy-4-methyl-phenoxy)-phenyl]-thiazolidine-4-carboxylic acid methyl ester

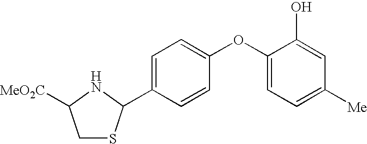

To a stirred suspension of 4-(2-hydroxy-4-methyl-phenoxy)-benzaldehyde (100 mg, 0.438 mmol) and cysteine methyl ester hydrochloride (77 mg, 0.438 mmol) in DCM (2 mL) was added triethylamine (0.067 mL) dropwise. After being stirred at ambient temperature overnight, the reaction mixture was diluted with DCM (10 mL) and washed with water (5 mL×2). The organic layer (suspension) was dried on anhydrous sodium sulfate, filtered and evaporated. The residue was purified by flash chromatography on silica column eluting with 5% of ethyl acetate in DCM, giving the Example title compound (85 mg, 56%) as white powder. M.P.: 134-135° C.; C₁₈H₁₉NO₄S (345.1035): HRMS (EI+) m/e: 345.1028. This product was analyzed by ¹H-NMR. The corresponding ¹H-NMR spectrum was consistent with the structure of the anticipated product consisting of a pair of diastereomers.

### Example 7

### 2-[4-(2-Hydroxy-4-methyl-phenoxy)-phenyl]-thiazolidine-4-carboxylic acid

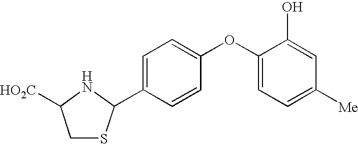

A solution of 4-(2-hydroxy-4-methyl-phenoxy)-benzaldehyde (75 mg, 0.329 mmol) and cysteine (44 mg, 0.362 mmol) in ethanol (1 mL) and water (1 mL) was stirred at ambient temperature overnight. The clear solution turned into a suspension. Water (3 mL) was added and the precipitate was collected by filtration under reduced pressure, and dried in vacuum. The Example title compound (97 mg, 89%) was obtained as white powder. M.P.: 172-174° C. (dec.); C₁₇H₁₇NO₄S+H (332.0957): MS (EI+) m/e: 331; HRMS (electron spray (ES)+) m/e: 332.0969 (M+H). This product was also analyzed by ¹H-NMR. The corresponding ¹H-NMR spectrum was consistent with the structure of the anticipated product consisting of a pair of diastereomers.

### Example 8

### 2-[4-(2-Hydroxy-4-methyl-phenoxy)-phenyl]-[1, 3]thiazinane-4-carboxylic acid

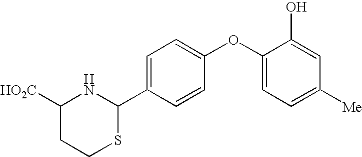

A solution of 4-(2-hydroxy-4-methyl-phenoxy)-benzaldehyde (75 mg, 0.329 mmol) and homocysteine (52 mg, 0.362 mmol) in ethanol (1 mL) and water (1 mL) was stirred at ambient temperature overnight. The clear solution turned into a suspension of precipitate. Water (3 mL) was added and the precipitate was collected by filtration under reduced pressure, and dried in vacuum. The Example title compound (113 mg, 99%) was obtained as white powder. M.P.: 140-145° C. (dec.); C₁₈H₁₉NO₄S+H (346.1113): HRMS (ES+) m/e: 346.1114 (M+H). This product was analyzed by ¹H-NMR. The corresponding ¹H-NMR spectrum was consistent with the structure of the anticipated product consisting of a pair of diastereomers.

### Example 9

### 2-[3-Cyano-4-(2-hydroxy-4-methyl-phenoxy)-phenyl]-thiazolidine-4-carboxylic acid

### Step 1: 5-Formyl-2-(2-methoxy-4-methyl-phenoxy)-benzonitrile

A suspension of 5-formyl-2-fluoro-benzonitrile (375 mg, 2.440 mmol), 2-methoxy-4-methyl-phenol (337 mg, 2.440 mmol) and cesium carbonate (874 mg, 2.680 mmol) in acetonitrile (20 mL) was stirred at ambient temperature overnight. The reaction mixture was poured into water and extracted with ether (30 mL×2). The organic layer was washed with brine (30 mL), dried over anhydrous sodium sulfate, filtered and evaporated. The crude solid residue was triturated in hexane and dried in vacuum. The final product (580 mg, 89%) was obtained as white powder. M.P.: 112-113° C.; C₁₆H₁₃NO₃ (267.09): GC-MS m/e 267.

### Step 2: 5-Formyl-2-(2-hydroxy-4-methyl-phenoxy)-benzonitrile

To a solution of 5-formyl-2-(2-methoxy-4-methyl-phenoxy)-benzonitrile (550 mg, 2.060 mmol) in DCM (10 mL), cooled on an acetone-dry ice bath, was added dropwise BBr₃ (0.600 mL, 6.180 mmol) via syringe. The reaction mixture was allowed to warm up to ambient temperature and stirred for 5 hrs. The mixture was quenched with methanol (1 mL), subsequently brine (10 mL), and extracted with DCM (10 ml×3). The combined organic layer was washed with saturated sodium chloride solution, dried on anhydrous sodium sulfate, filtered and evaporated. The residue was chromatographed on silica gel eluting with 2% of methanol in DCM. The final product (260 mg, 50%) was obtained as white powder. M.P.: 130-133° C. (dec.); C₁₅H₁₁NO₃ (253.07): (GC-MS) m/e 253.

### Step 3: 2-[3-Cyano-4-(2-hydroxy-4-methyl-phenoxy)-phenyl]-thiazolidine-4-carboxylic acid

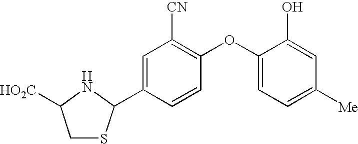

A solution of 5-formyl-2-(2-hydroxy-4-methyl-phenoxy)-benzonitrile (60 mg, 0.237 mmol) and cysteine (33 mg, 0.261 mmol) in ethanol (1 mL) and water (1 mL) was stirred at ambient temperature overnight. The clear solution turned into a suspension of precipitate. Water (3 mL) was added and the precipitate was collected by filtration under reduced pressure, and dried in vacuum. The Example title compound (50 mg, 59%) was obtained as white powder. M.P. 150-152° C.; C₁₈H₁₆N₂O₄S+H (357.0909): HRMS (ES+) m/e: 357.0899 (M+H). The product was analyzed by ¹H-NMR. The corresponding ¹H-NMR spectrum was consistent with the structure of the anticipated product consisting of a pair of diastereomers.

### Example 10

### 2-[3-Cyano-4-(2-hydroxy-4-methyl-phenoxy)-phenyl]-[1,3]thiazinane-4-carboxylic acid

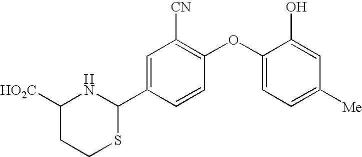

A solution of 5-formyl-2-(2-hydroxy-4-methyl-phenoxy)-benzonitrile (60 mg, 0.237 mmol) and homocysteine (37 mg, 0.261 mmol) in ethanol (1 mL) and water (1 mL) was stirred at ambient temperature overnight. The clear solution turned into a suspension of precipitate. Water (3 mL) was added and the precipitate was collected by filtration under reduced pressure, and dried in vacuum. The Example title compound (70 mg, 80%) was obtained as white powder. M.P.: 147-150° C. (dec.); C₁₉H₁₈N₂O₄S+H (371.1066): HRMS (ES+) m/e: 371.1048 (M+H). This product was analyzed by ¹H-NMR. The corresponding ¹H-NMR spectrum was consistent with the structure of the anticipated product consisting of a pair of diastereomers.

### Example 11

### 5-Methyl-2-[4-(pyrrol-1-yl)-phenoxy]-phenol

### Step 1: 5-Methyl-2-(4-nitro-phenoxy)-anisole

A suspension of 1-fluoro-4-nitro-benzene (1.022 g, 7.24 mmol), 2-methoxy-4-methyl-phenol (1.000 g, 7.24 mmol) and cesium carbonate (2.830 g, 8.690 mmol) in DMA (10 mL) was stirred at 100° C. overnight. The reaction mixture was poured into water and extracted with ether (20 mL×3). The organic layer was washed with water (30 mL), dried over anhydrous sodium sulfate, filtered and evaporated. The crude solid residue was triturated in hexane and dried in vacuum. The final product (1.590 g, 85%) was obtained as white powder. M.P.: 78-80° C.

### Step 2: 5-Methyl-2-(4-nitro-phenoxy)-phenol

To a solution of 5-methyl-2-(4-nitro-phenoxy)-anisole (1.000 g, 3.860 mmol) in DCM (10 mL), cooled on an acetone-dry ice bath was added dropwise BBr₃ (0.365 mL, 3.860 mmol) via syringe. The cooling bath was removed and the reaction mixture was stirred at ambient temperature for 5 hrs. The mixture was quenched with methanol (1 mL), subsequently brine (5 mL), and extracted with DCM (10 ml×3). The combined organic layer was washed with brine, dried on anhydrous sodium sulfate, filtered and evaporated. The solid residue was further washed with hexane and dried in vacuum to give the Example title compound (827 mg, 87%) as yellowish crystal. M.P.: 95-97° C.; C₁₃H₁₁NO₄ (245.07): GC-MS (EI+) m/e: 245. The product was analyzed by ¹H-NMR. The corresponding ¹H-NMR spectrum was consistent with the structure of the anticipated product.

### Step 3: 5-Methyl-2-[4-(pyrrol-1-yl)-phenoxy]-phenol

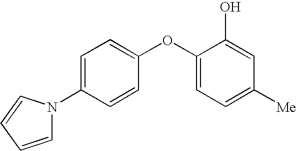

A suspension of 5-methyl-2-(4-nitro-phenoxy)-phenol (100 mg, 0.408 mmol) and 10% palladium on charcoal (45 mg) in methanol was stirred vigorously for 30 min in a hydrogen atmosphere provided with a hydrogen balloon and evaporated under reduced pressure. The residue, together with 2,5-dimethoxy-tetrahydro-furan (0.058 mL, 0.449 mmol) was dissolved in acetic acid (1 mL) and heated at 100° C. overnight. The reaction mixture was poured into water and extracted with DCM (10 mL×3). The organic layer was washed with saturated aqueous sodium carbonate solution, dried over anhydrous sodium sulfate, filtered through a short column of celite and evaporated. The solid residue was triturated in hexane and dried in vacuum to give the Example title compound (59 mg, 55%) as white powder. M.P.: 95-96° C.; C₁₇H₁₅NO₂ (265.1103): HRMS (EI+) m/e: 265.1109. The product was analyzed by ¹H-NMR. The corresponding ¹H-NMR spectrum was consistent with the structure of the anticipated product.

### Example 12

### 5-(Furan-2-yl)-2-(4-fluoro-2-hydroxy-phenoxy)-phenol

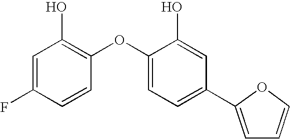

The Example title compound was obtained by following the same procedure as described in Example 4 with 5-bromo-2-(4-fluoro-2-hydroxyphenoxy)phenol as starting material. M.P.: 102-104° C.

### Example 13

### 5-Methyl-2-[4-(1H-tetrazol-5-yl)-phenoxy]-phenol and 5-methyl-2-[4-(2H-tetrazol-5-yl)-phenoxy]-phenol

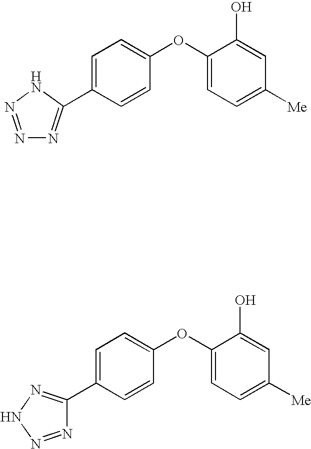

### Step 1: 4-(2-Methoxy-4-methyl-phenoxy)-benzonitrile

A suspension of 4-fluoro-benzonitrile (2.102 g, 17.36 mmol), 2-methoxy-4-methyl-phenol (2.000 g, 14.47 mmol) and cesium carbonate (5.186 g, 15.92 mmol) in DMA was vigorously stirred at 100° C. for 30 hrs. The reaction mixture was poured into water, and extracted with ether (20 mL×3). The organic layer was washed with water, dried over anhydrous sodium sulfate, filtered and evaporated. The crude residue (3.452 g, 99%) was used for the next reaction without further purification.

### Step 2: 4-(2-Hydroxy-4-methyl-phenoxy)-benzonitrile

To a solution of 4-(2-methoxy-4-methyl-phenoxy)-benzonitrile (3.542 g, 14.43 mmol) in DCM (50 mL), cooled on an acetone-dry ice bath, was added dropwise boron tribromide (2.739 mL, 28.85 mmol) via syringe. The cooling bath was removed and the reaction was allowed to warm up to ambient temperature. After being stirred for 5 hrs, the reaction mixture was quenched by adding methanol (1 mL) dropwise, followed by water (20 mL). The organic layer was separated and the aqueous layer was extracted with DCM (20 mL×3). The combined organic phase was washed with brine, dried over anhydrous sodium sulfate, filtered and evaporated. The solid residue was washed with hexane and dried in vacuum to give the Example title compound (2.660 g, 82%) as white powder. M.P.: 118-119° C.; C₁₄H₁₁NO₂ (225.08): GC-MS (EI+) m/e: 225. This product was analyzed by ¹H-NMR. The corresponding ¹H-NMR spectrum was consistent with the structure of the anticipated product.

### Step 3: 5-Methyl-2-[4-(1H-tetrazol-5-yl)-phenoxy]-phenol and 5-methyl-2-[4-(2H-tetrazol-5-yl)-phenoxy]-phenol

A suspension of 4-(2-hydroxy-4-methyl-phenoxy)-benzonitrile (200 mg, 0.890 mmol), sodium azide (116 mg, 1.780 mmol) zinc bromide (102 mg, 0.445 mmol) in isopropanol (1.5 mL) and water (3.0 mL) was refluxed for 16 hrs. The reaction mixture was diluted with water (10 mL), acidified with 3N hydrochloric acid (0.5 mL) and extracted with ethyl acetate (5 mL×3). The organic layer was washed with brine, dried on anhydrous sodium sulfate and concentrated. The solid residue was triturated in 50% DCM in hexane and dried in vacuum. The final product (168 mg, 70%) was obtained as white powder. M.P.:173-175° C.; C₁₄H₁₂N₄O₂+H (269.1039): HRMS (ES+) m/e: 269.1034. This product was analyzed by ¹H-NMR. The corresponding ¹H-NMR spectrum was consistent with the structure of the anticipated product consisting of a pair of tautomers.

### Example 14

### 5-Methyl-2-[2-(1H-tetrazol-5-yl)-phenoxy]-phenol and 5-methyl-2-[2-(2H-tetrazol-5-yl)-phenoxy]-phenol

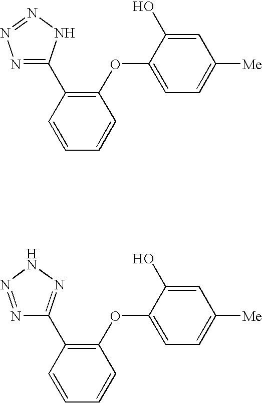

A suspension of 2-(2-hydroxy-4-methyl-phenoxy)-benzonitrile (200 mg, 0.890 mmol), sodium azide (116 mg, 1.780 mmol), zinc bromide (102 mg, 0.445 mmol) in isopropanol (1.5 mL) and water (3.0 mL) was refluxed for 16 hrs. The reaction mixture was diluted with water (10 mL), acidified with 3N hydrochloric acid (0.5 mL) and extracted with ethyl acetate (5 mL×3). The organic layer was washed with brine, dried on anhydrous sodium sulfate and concentrated. The solid residue was triturated in 50% DCM in hexane and dried in vacuum. The final product (170 mg, 71%) was obtained as white powder. M.P.: 205-207° C.; C₁₄H₁₂N₄O₃+H (269.1039): HRMS (ES+) m/e: 269.1047. This product was analyzed by ¹H-NMR. The corresponding ¹H-NMR spectrum was consistent with the structure of the anticipated product consisting of a pair of tautomers.

Examples 15-21 were made by following the procedure described in Example 14.

### Example 15

### 5-Chloro-2-[2-(1H-tetrazol-5-yl)-phenoxy]-phenol and 5-chloro-2-[2-(2H-tetrazol-5-yl)-phenoxy]-phenol

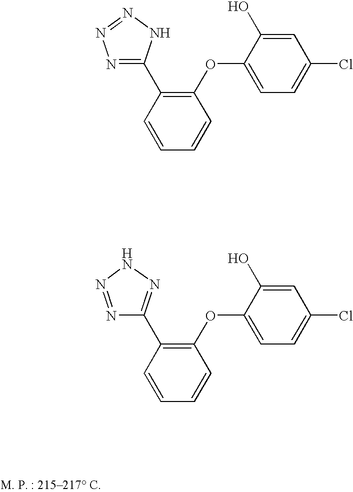

### Example 16

### 5-Methyl-2-[4-nitro-2-(1H-tetrazol-5-yl)-phenoxy]-phenol and 5-methyl-2-[4-nitro-2-(2H-tetrazol-5-yl)-phenoxy]-phenol

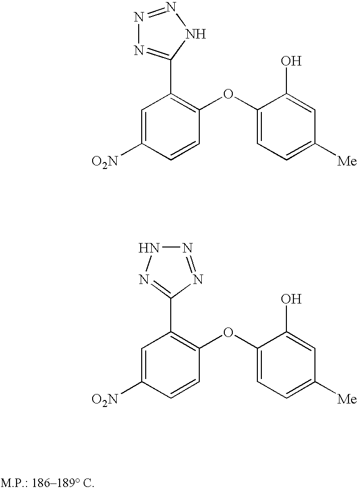

### Example 17

### 5-Fluoro-2-[2-(1H-tetrazol-5-yl)-phenoxy]-phenol and 5-fluoro-2-[2-(2H-tetrazol-5-yl)-phenoxy]-phenol

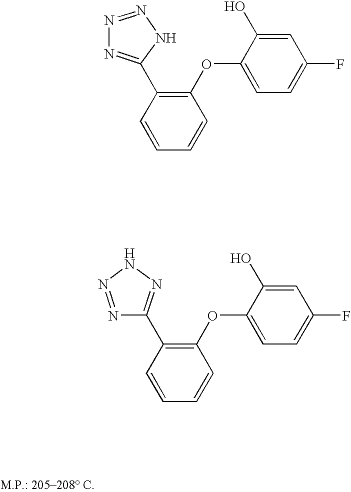

### Example 18

### 2-[4-Chloro-2-(1H-tetrazol-5-yl)-phenoxy]-5-fluoro-phenol and 2-[4-chloro-2-(2H-tetrazol-5-yl)-phenoxy]-5-fluoro-phenol

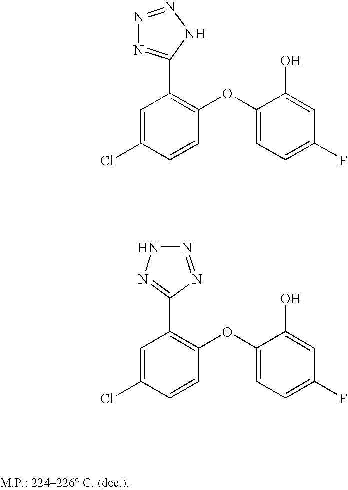

### Example 19

### 5-chloro-2-[4-Chloro-2-(1H-tetrazol-5-yl)-phenoxy]-phenol and 5-chloro-2-[4-chloro-2-(2H-tetrazol-5-yl)-phenoxy]-phenol

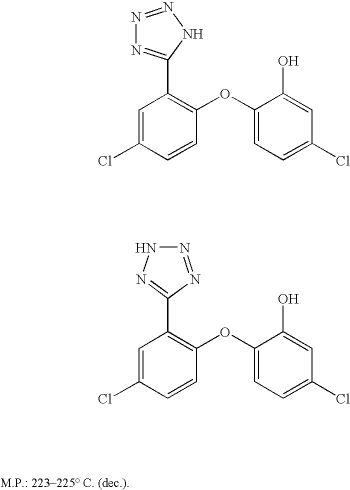

### Example 20

### 2-[2-(1H-Tetrazol-5-yl)-phenoxy]-phenol and 2-[2-(2H-tetrazol-5-yl)-phenoxy]-phenol

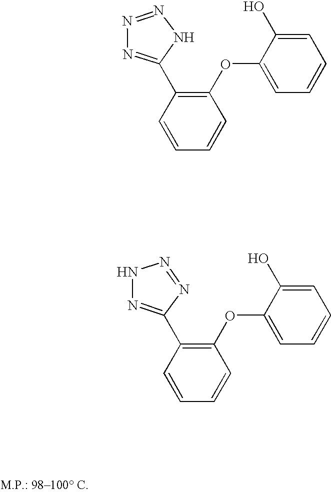

### Example 21

### 4-Bromo-2-[2-(1H-tetrazol-5-yl)-phenoxy]-phenoll and 4-bromo-2-[2-(2H-tetrazol-5-yl)-phenoxy]-phenol

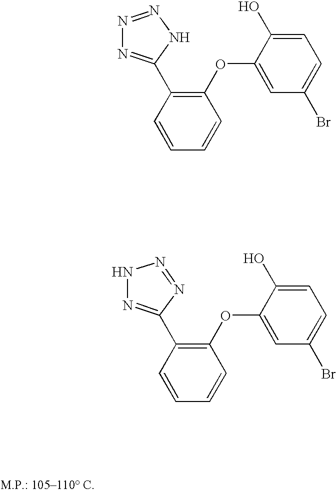

### Example 22

### 2-[2-(5-Amino-[1,3,4]thiadiazol-2-yl)-phenoxy]-5-fluoro-phenol

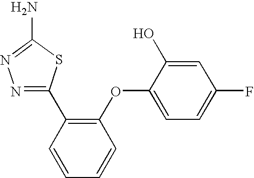

A pressure bottle, charged with a solution of semicarbazide hydrochloride (41 mg, 0.445 mmol), 2-(4-fluoro-2-hydroxy-phenoxy)-benzonitrile (102 mg, 0.445 mmol) in TFA (1 mL) was heated at 70° C. for 28 hrs. The reaction mixture was poured into water (10 mL), neutralized with saturated aqueous sodium carbonate solution and mixed with DCM. The suspension was filtered and the solid residue was collected and dried in vacuum. The product (38 mg, 28%) was obtained as white powder. M.P.: 250-255° C. (dec.); C₁₄H₁₀FN₃O₂S+H (304.0556): HRMS (ES+) m/e: 304.0562 (M+H). This product was analyzed by ¹H-NMR. The corresponding ¹H-NMR spectrum was consistent with the structure of the anticipated product.

### Example 23

### 2-[4-(5-Amino-[1,3,4]thiadiazol-2-yl)-phenoxy]-5-methyl-phenol

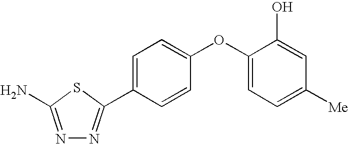

The Example title compound was synthesized from the corresponding cyano compound by following the same procedure as described in Example 23. M.P.: 240-243° C. (dec.); C₁₅H₁₃N₃O₂S+H (300.08): MS (ES+) m/e: 300 (M+H). This product was analyzed by ¹H-NMR. The corresponding ¹H-NMR spectrum was consistent with the structure of the anticipated product.

### Example 24

### 2-[4-(5-Amino-[1,3,4]oxadiazol-2-yl)-phenoxy]-5-methyl-phenol

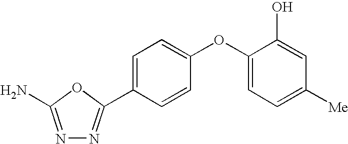

The Example title compound was synthesized by following Example 23 using semicarbazide instead of thiosemicarbazide. M.P.: 181-183° C.

### Example 25

### 2-(4-Fluoro-2-hydroxy)-N-hydroxy-benzamidine

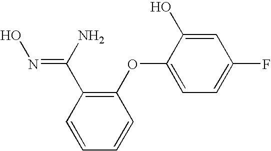

A stirred solution of 2-(4-fluoro-2-hydroxy-phenoxy)-benzonitrile (100 mg, 0.44 mmol), hydroxylamine hydrochloride (60.6 mg 0.87 mmol) and sodium carbonate (46.6 mg 0.44 mmol) in ethanol (0.5 mL) and water (0.25 mL) was refluxed overnight. The reaction mixture was evaporated under reduced pressure to completely remove ethanol and water. The crude residue was chromatographed on a silica column eluting with 2.5% MeOH in DCM, giving the product as white crystal (79 mg, 69.7%). M.P.: 64-65° C.; C₁₃H₁₁FN₂O₃ (262.0754): HRMS (EI+) m/e: 262.0749. This product was analyzed by ¹H-NMR. The corresponding ¹H-NMR spectrum was consistent with the structure of the anticipated product.

Examples 26-30 were synthesized employing corresponding cyano compounds as starting materials by following the same procedure as described in Example 25.

### Example 26

### N-Hydroxy-2-(2-hydroxy-4-methyl-phenoxy)-benzamidine

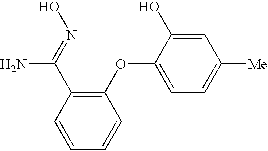

M.P.: 85-87° C. This product was analyzed by ¹H-NMR. The corresponding ¹H-NMR spectrum was consistent with the structure of the anticipated product.

### Example 27

### 5-Chloro-2-(4-fluoro-2-hydroxy)-N-hydroxy-benzamidine

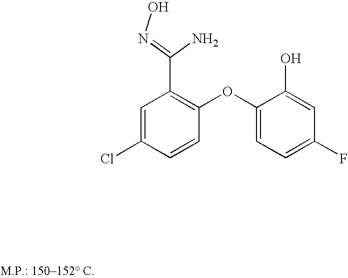

### Example 28

### N-Hydroxy-2-(2-hydroxy-4-methyl-phenoxy)-5-nitro-benzamidine

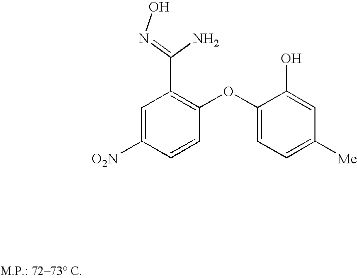

### Example 29

### 5-Chloro-2-(4-chloro-2-hydroxy-phenoxy)-N-hydroxy-benzamidine

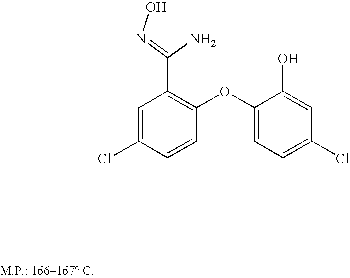

### Example 30

### N-Hydroxy-4-(2-hydroxy-4-methyl-phenoxy)-benzamidine

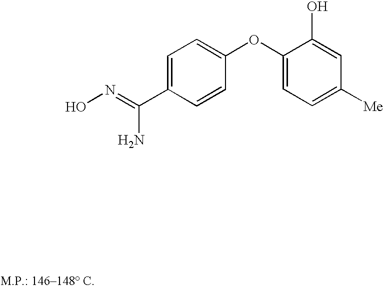

### Example 31

### 3-[2-(4-Fluoro-2-hydroxy)-phenyl]-[1,2,4]oxadiazole-5-carboxylic acid ethyl ester

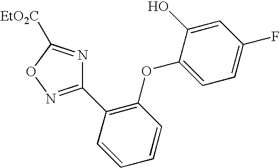

To a stirred solution of 2-(4-fluoro-2-hydroxy)-N-hydroxy-benzamidine (320 mg, 1.22 mmol), pyridine (0.25 ml) in DCM (5.25 ml) was added ethyl chlorooxoacetate (0.171 ml, 1.54 mmol) by syringe. After being refluxed for 6 hours, the reaction mixture was quenched with 10 ml of saturated NH₄Cl solution and stirred for another 20 minutes. The reaction mixture was extracted with DCM and the organic layer was washed with saturated NaHCO₃ solution, dried over Na₂SO₄, filtered and evaporated. Flash chromatography on silica column eluted with 2.5% methanol in DCM provided the Example title compound as white crystal (256 mg 62%). M.P.: 82-83° C.; C₁₇H₁₃FN₂O₅ (344.0808): HRMS (EI+) m/e: 344.0812. This product was analyzed by ¹H-NMR. The corresponding ¹H-NMR spectrum was consistent with the structure of the anticipated product.

Examples 32-35 were synthesized using corresponding N-hydroxy benzamidines as starting materials by following the same procedure as described in Example 31.

### Example 32

### 3-[2-(2-Hydroxy-4-methyl-phenoxy)-phenyl]-[1,2,4]oxadiazole-5-carboxylic acid ethyl ester

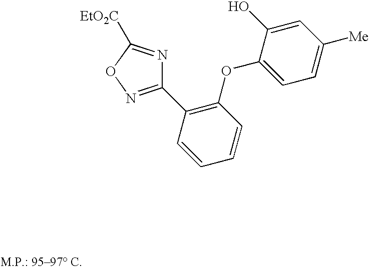

### Example 33

### 3-[5-Chloro-2-(4-fluoro-2-hydroxy-phenoxy)-phenyl]-[1,2,4]oxadiazole-5-carboxylic acid ethyl ester

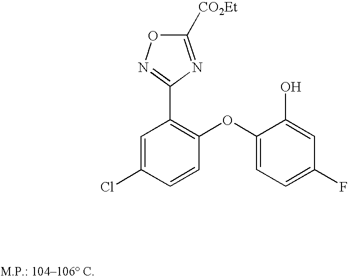

### Example 34

### 3-[2-(2-Hydroxy-4-methyl-phenoxy)-5-nitro-phenyl]-[1,2,4]oxadiazole-5-carboxylic acid ethyl ester

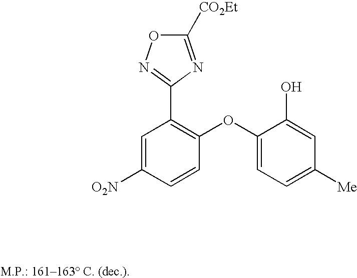

### Example 35

### 3-[4-(2-Hydroxy-4-methyl-phenoxy)-phenyl]-[1,2,4]oxadiazole-5-carboxylic acid ethyl ester

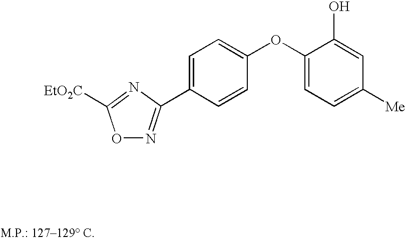

### Example 36

### 3-[2-(4-Fluoro-2-hydroxy-phenoxy)-phenyl]-[1,2,4]oxadiazole-5-carboxylic acid

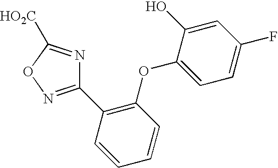

A solution of 3-[2-(4-fluoro-2-hydroxy)-phenyl]-[1,2,4]oxadiazole-5-carboxylic acid ethyl ester (100 mg, 0.29 mmol), LiOH (17.5 mg, 0.73 mmol) in tetrahydrofuran (THF) (0.65 ml) and water (0.65 ml) was stirred at 30° C. for 30 min. The reaction mixture was acidified to pH 4 with 1N hydrochloric acid, diluted with water (4 ml) and extracted with ethyl acetate (5 mL×3). The ethyl acetate phase was washed with brine and dried over sodium sulfate. The crude residue was purified by flash chromatography on a silica column eluted with 2.5% methanol in DCM. The Example title compound was obtained as white crystal (41 mg, 45%). M.P.: 127-128° C.; C₁₅H₉FN₂O₅ (316.05): MS (EI+) m/e: 272 (M−CO₂). The corresponding ¹H-NMR spectrum was consistent with structure of the anticipated product.

Examples 37-40 were synthesized using corresponding esters as starting materials by following the same procedure as described in Example 36.

### Example 37

### 3-[2-(2-Hydroxy-4-methyl-phenoxy)-phenyl]-[1,2,4]oxadiazole-5-carboxylic acid

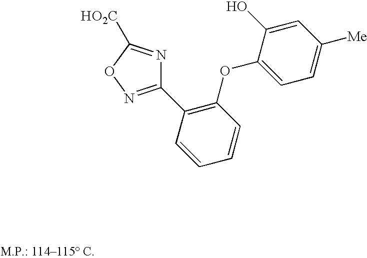

### Example 38

### 3-[5-Chloro-2-(4-fluoro-2-hydroxy-phenoxy)-phenyl]-[1,2,4]oxadiazole-5-carboxylic acid

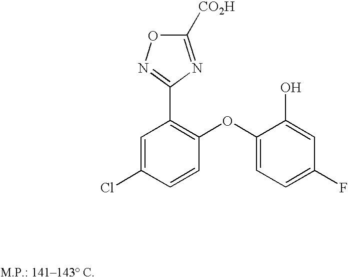

### Example 39

### 3-[2-(2-Hydroxy-4-methyl-phenoxy)-5-nitro-phenyl]-[1,2,4]oxadiazole-5-carboxylic acid

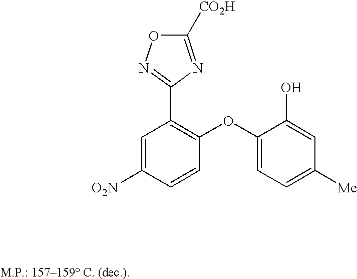

### Example 40

### 3-[4-(2-Hydroxy-4-methyl-phenoxy)-phenyl]-[1,2,4]oxadiazole-5-carboxylic acid

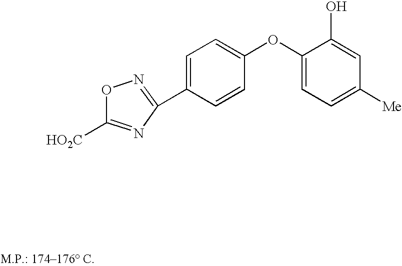

### Example 41

### 5-Methyl-2-(4-nitro-2-[1,2,4]oxadiazol-3-yl-phenoxy)-phenol

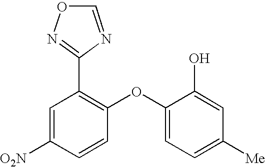

A solution of N-hydroxy-2-(2-hydroxy-4-methyl-phenoxy)-5-nitro-benzamidine (100 mg, 0.33 mmol) in triethyl orthoformate (1.5 ml) was refluxed overnight. The reaction mixture was evaporated under reduced pressure and the residue was purified by flash chromatography on silica column eluted with 30% of ethyl acetate in hexane. The Example title compound was obtained as yellowish crystal (70 mg, 68%). M.P.: 159-160° C.; C₁₅H₁₁N₃O₅ (313.0699): HRMS (EI+) m/e: 313.0701. The corresponding ¹H-NMR spectrum was consistent with the structure of anticipated product.

Examples 42-45 were synthesized employing corresponding N-hydroxy benzamidines as starting materials by following the same procedure as described in Example 41.

### Example 42

### 5-Fluoro-2-(2-[1,2,4]oxadiazol-3-yl-phenoxy)-phenol

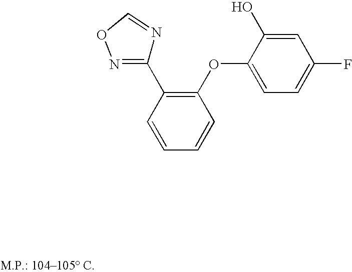

### Example 43

### 5-Methyl-2-(2-[1,2,4]oxadiazol-3-yl-phenoxy)-phenol

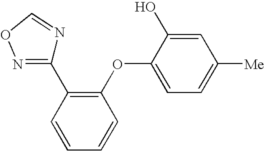 R_{f} (ethyl acetate:hexane=15%): 0.23; C₁₅H₁₂N₂O₃ (268.08): GC-MS m/e 268; The corresponding ¹H-NMR spectrum was consistent with the structure of anticipated product.

### Example 44

### 2-(4-Chloro-2-[1,2,4]oxadiazol-3-yl-phenoxy)-5-fluoro-phenol

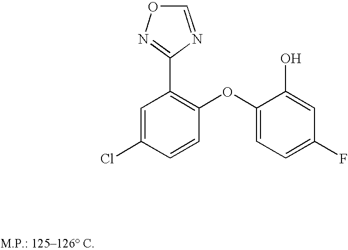

### Example 45

### 5-Methyl-2-(4-[1,2,4]oxadiazol-3-yl-phenoxy)-phenol

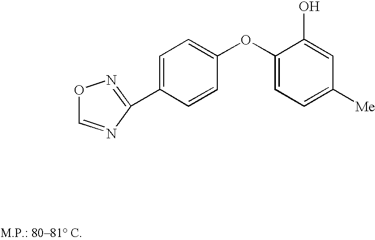

### Example 46

### 3-[2-(2-Hydroxy-4-methyl-phenoxy)-phenyl]-4H-[1,2,4]oxadiazol-5-one

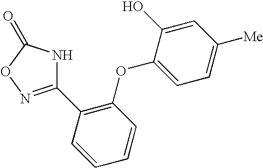

A stirred solution of N-hydroxy-2-(2-hydroxy-4-methyl-phenoxy)-benzamidine (100 mg, 0.387 mmol), and carbonyl dimidazole (75 mg, 0.464 mmol) in THF (1.5 mL) was refluxed for 4 hrs. The solvent was removed and the residue was chromatographed on a silica column eluting with 5% methanol in DCM, giving the Example title compound (81 mg, 74%) as white powder. M.P.: 213-214° C.; C₁₅H₁₂N₂O₄+H (285.0875): HRMS (ES+) m/e: 285.0886. This product was analyzed by ¹H-NMR. The corresponding ¹H-NMR spectrum was consistent with the structure of the anticipated product.

### Example 47

### 3-[4-(2-Hydroxy-4-methyl-phenoxy)-phenyl]-4H-[1,2,4]oxadiazol-5-one

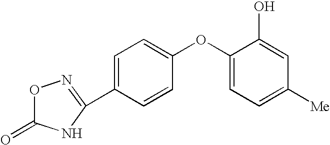

A stirred solution of N-hydroxy-4-(2-hydroxy-4-methyl-phenoxy)-benzamidine (100 mg, 0.387 mmol), and carbonyl dimidazole (75 mg, 0.464 mmol) in THF (2.0 mL) was refluxed for 4 hrs. The solvent was removed and the residue was chromatographed on a silica column eluting with 5% methanol in DCM, giving the Example title compound (66 mg, 60%) as white powder. M.P.: 195-198° C.; C₁₅H₁₂N₂O₄+H (285.0875): HRMS (ES+) m/e: 285.0869. The product was analyzed by ¹H-NMR. The corresponding ¹H-NMR spectrum was consistent with the structure of anticipated product.

Examples 48-50 were synthesized from corresponding cyano compounds by following the same procedure as described in Example 25.

### Example 48

### 2-(4-Chloro-2-hydroxy-phenoxy)-N-hydroxy-benzamidine

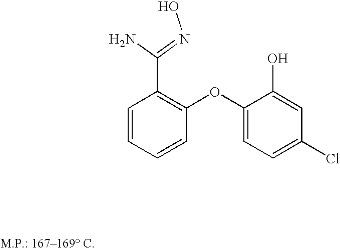

### Example 49

### N-Hydroxy-4-(2-hydroxy-phenoxy)-3-nitro-benzamidine

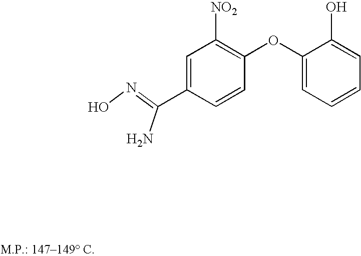

### Example 50

### 4-(4-Chloro-2-hydroxy-phenoxy)-N-hydroxy-3-nitro-benzamidine

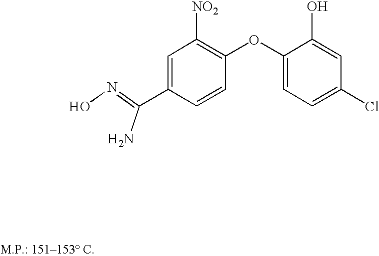

Example 51-53 were synthesized using Examples 48, 49 and 50 as starting materials by following the procedure as described in Example 31.

### Example 51

### 3-[2-(4-Chloro-2-hydroxy-phenoxy)-phenyl]-[1,2,4]oxadiazole-5-carboxylic acid ethyl ester

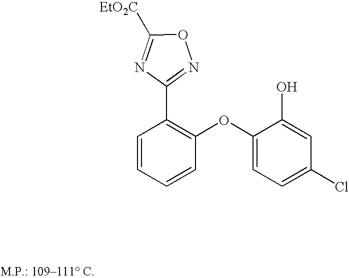

### Example 52

### 3-[4-(2-Hydroxy-phenoxy)-3-nitro-phenyl]-[1,2,4]oxadiazole-5-carboxylic acid ethyl ester

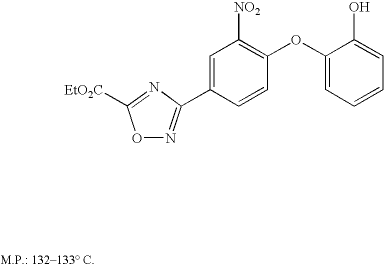

### Example 53

### 3-[4-(4-Chloro-2-hydroxy-phenoxy)-3-nitro-phenyl]-[1,2,4]oxadiazole-5-carboxylic acid ethyl ester

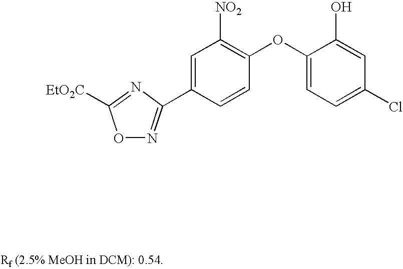 Example 54-56 were synthesized using Examples 51, 52 and 53 as starting materials by following the procedure as described in Example 36.

### Example 54

### 3-[2-(4-Chloro-2-hydroxy-phenoxy)-phenyl]-[1,2,4]oxadiazole-5-carboxylic acid

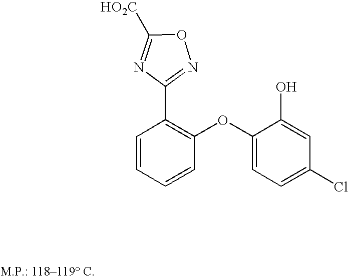

### Example 55

### 3-[4-(2-Hydroxy-phenoxy)-3-nitro-phenyl]-[1,2,4]oxadiazole-5-carboxylic acid

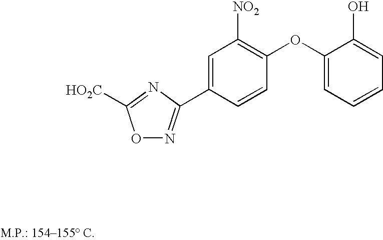

### Example 56

### 3-[4-(4-Chloro-2-hydroxy-phenoxy)-3-nitro-phenyl]-[1,2,4]oxadiazole-5-carboxylic acid

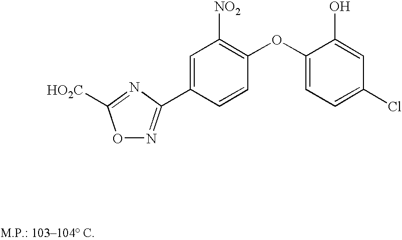

### Example 57

### 5-Chloro-2-(2-[1,2,4]oxadiazol-3-yl-phenoxy)-phenol

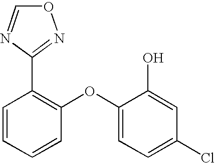

The Example title compound was obtained employing Example 48 as a starting material by following the procedure described in Example 41: M.P.: 124-125° C.

### Example 58

### 5-Methyl-2-(2-tetrazol-1-yl-phenoxy)-phenol

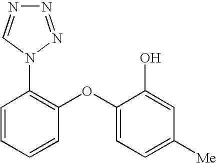

A suspension of 2-(2-amino-phenoxy)-5-methyl-phenol (215 mg, 1.0 mmol), sodium azide (98 mg, 1.5 mmol) and triethylorthoformate (237 mg, 1.6 mmol) in acetic acid was magnetically stirred at reflux for 6 hrs. The reaction mixture was poured into ice water (25 mL) and placed into icebox overnight. The solids were filtered, washed with water and dried. The resulting powder was washed with hexanes giving the Example title compound (234 mg, 87%) as a light brown powder: M.P.: 157-160° C. This product was analyzed by ¹H-NMR. The corresponding ¹H-NMR spectra was consistent with the structure of the anticipated product.

### Example 59

### 5-Methyl-2-(4-tetrazol-1-yl-phenoxy)-phenol

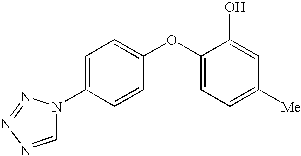

The Example title compound was obtained from 2-(4-amino-phenoxy)-5-methyl-phenol by following the procedure described in Example 58: M.P.: 129-132° C.

### Example 60

### N-Amino-4-(2-hydroxy-4-methyl-phenoxy)-benzamidine

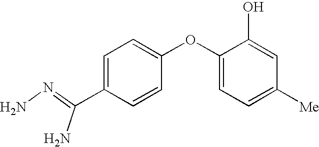

A solution of 4-(2-hydroxy-4-methyl-phenoxy)-benzonitrile (Example 13, Step 1) (500 mg, 2.22 mmol) and hydrazine (0.142 mL, 4.44 mmol) in ethanol (1 mL) was refluxed for 3 days. A thick suspension formed. The precipitate was collected by filtration, washed with cooled ethanol and dried in vacuum. The Example title compound (240 mg, 42%) was obtained as a white powder. M.P.:>260° C.

### Example 61

### N-Amino-2-(2-hydroxy-4-methyl-phenoxy)-benzamidine

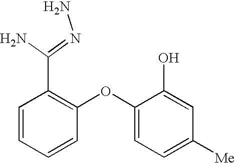

The Example title compound was obtained employing 2-(2-hydroxy-4-methyl-phenoxy)-benzonitrile as a starting material by following the procedure described in Example 60. M.P.:>260° C.

### Example 62

### 1-[2-(2-Hydroxy-4-methyl-phenoxy)-phenyl]-1H-[1,2,3]triazole-4-carboxylic acid ethyl ester

### Step 1: 2-(2-Azido-phenoxy)-5-methyl-phenol

To a stirred suspension of 2-(2-amino-phenoxy)-5-methyl-phenol (200 mg, 0.93 mmol) in water (1.25 mL) was added concentrated hydrochloric acid (1.25 mL). The solution was cooled on a salted water-ice bath (−10° C.) and sodium nitrite (71 mg, 1.023 mmol) was added. After being stirred for two hours at temperature between −10-0° C., a cooled solution of sodium azide (122 mg, 1.86 mmol) and sodium acetate (1.526 g) in water (3 mL) was added. After being stirred for another hour, the reaction mixture was extracted with ethyl acetate (3×5 mL). The organic layer was washed with saturated sodium bicarbonate solution, water, dried over sodium sulfate, filtered and evaporated. The residue was purified by flash chromatography on a silica column eluted with 15% AcOEt in hexane. The product (91 mg, 41%) was obtained as yellowish oil: R_{f} (AcOEt/hexane: 15%): 0.43; C₁₃H₁₁N₃O₂ (241.09): GC-MS (EI+) m/e: 215 (M⁺−2N). This product was analyzed by ¹H-NMR. The corresponding ¹H-NMR spectrum was consistent with the structure of the anticipated product.

### Step 2: 1-[2-(2-Hydroxy-4-methyl-phenoxy)-phenyl]-1H-[1,2,3]triazole-4-carboxylic acid methyl ester

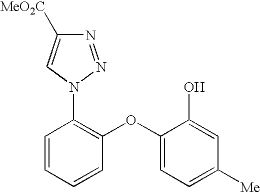

A stirred solution of 2-(2-azido-phenoxy)-5-methyl-phenol (80 mg, 0.35 mmol) and methyl propiolate (0.45 mL, 1.74 mmol) in toluene (1 mL) was heated at 100° C. overnight. The reaction mixture was evaporated under reduced pressure and the residue was purified by flush chromatography on a silica column eluted with a gradient solvent of AcOEt in DCM from 5 to 15%. The Example title compound (68 mg, 60%) was obtained as a white powder: M.P.: 153-155° C.; C₁₇H₁₅N₃O₄ (325.11): GC-MS (EI+) m/e: 325. This product was analyzed by ¹H-NMR. The corresponding ¹H-NMR spectrum was consistent with the structure of the anticipated product.

### Example 63

### 1-[4-(2-Hydroxy-4-methyl-phenoxy)-phenyl]-1H-[1,2,3]triazole-4-carboxylic acid ethyl ester

### Step 1: 2-(4-Azido-phenoxy)-5-methyl-phenol

The Example title compound (159 mg, 71%) was obtained as an oil from 2-(4-amino-phenoxy)-5-methyl-phenol by following the procedure described in Example 62, Step 1: R_{f} (AcOEt/hexane: 15%): 0.31; C₁₃H₁₁N₃O₂ (241.09): GC-MS (EI+) m/e: 215 (M⁺−2N). This product was analyzed by ¹H-NMR. The corresponding ¹H-NMR spectrum was consistent with the structure of the anticipated product.

### Step 2: 1-[4-(2-Hydroxy-4-methyl-phenoxy)-phenyl]-1H-[1,2,3]triazole-4-carboxylic acid methyl ester

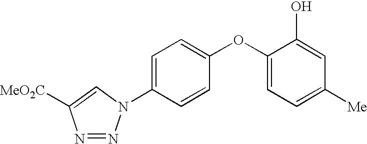

The Example title compound (145 mg, 83%) was obtained as a white powder from 2-(4-azido-phenoxy)-5-methyl-phenol by following the same procedure as described in Example 62, Step 2: M.P.: 148-151° C.; C₁₇H₁₅N₃O₄ (325.11): GC-MS (EI+) m/e: 267(M⁺−CO₂Me). This product was analyzed by ¹H-NMR. The corresponding ¹H-NMR spectrum was consistent with the structure of the anticipated product.

### Example 64

### 1-[2-(2-Hydroxy-4-methyl-phenoxy)-phenyl]-1H-[1,2,3]triazole-4-carboxylic acid

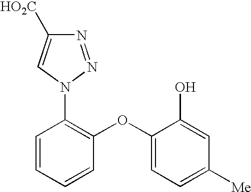

A suspension of 1-[2-(2-hydroxy-4-methyl-phenoxy)-phenyl]-1H-[1,2,3]triazole-4-carboxylic acid methyl ester (Example 62) (80 mg, 0.246 mmol) and lithium hydroxide (12 mg, 0.5 mmol) in methanol (0.75 mL) and water (0.75 mL) was stirred at ambient temperature for 4 hr. The reaction mixture was neutralized with 1N hydrochloric acid to pH 3.0 while cooled on an ice-water bath and extracted with ethyl acetate (3×10 mL). The organic layer was washed with brine, dried over magnesium sulfate, filtered and evaporated. The solid residue was triturated in DCM/hexane (1:9) and dried in vacuum, giving the Example title compound (70 mg, 91%) as a white powder: M.P.: 168-169° C. This product was analyzed by ¹H-NMR. The corresponding ¹H-NMR spectrum was consistent with the structure of the anticipated product.

### Example 66

### 1-[4-(2-Hydroxy-4-methyl-phenoxy)-phenyl]-1H-[1,2,3]triazole-4-carboxylic acid

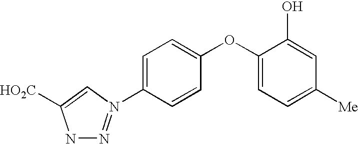

The Example title compound (38 mg, 79%) was obtained from 1-[4-(2-hydroxy-4-methyl-phenoxy)-phenyl]-1H-[1,2,3]triazole-4-carboxylic acid methyl ester (Example 63) by following the same procedure as described in Example 64: M.P.: 159-161° C. This product was analyzed by ¹H-NMR. The corresponding ¹H-NMR spectrum was consistent with the structure of the anticipated product.

### Example 66

### 2-(2-Hydrazino-phenoxy)-5-methyl-phenol

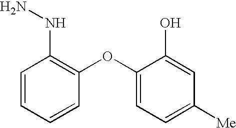

To a stirred suspension of 2-(2-amino-phenoxy)-5-methyl-phenol (500 mg, 2.323 mmol) in methanol (1 mL), water (2 mL) and concentrated hydrochloric acid (0.6 mL), cooled on a salted water-ice bath (−10° C.), was added a solution of sodium nitrite (168 mg, 2.439 mmol) in water (0.4 mL). After being stirred for 30 min., the reaction mixture was transferred by pipette to a stirred solution of SnCl₂.2H₂O (1.253 g, 9.974 mmol) in concentrated hydrochloric acid (5 mL) at −10° C. The reaction mixture was stirred for 4 hrs at the temperature between −10 to 0° C., combined with methanol (5 mL), neutralized with 10 N sodium hydroxide to pH 7-8. The milky suspension was evaporated under reduced pressure and in vacuum to remove the solvent and water completely. The solid residue was triturated with 10% MeOH/DCM and filtered. The filtrate was evaporated and the solid residue was washed with 20% DCM/hexane and dried in vacuum, giving the Example title compound (297 mg, 56%) as reddish solid: M.P.: 108-110° C. This product was analyzed by ¹H-NMR. The corresponding ¹H-NMR spectrum was consistent with the structure of the anticipated product.

### Example 67

### 2-(4-Hydrazino-phenoxy)-5-methyl-phenol

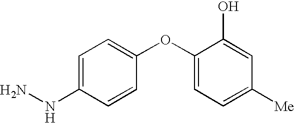

The Example title compound (876 mg, 67%) was obtained from 2-(4-amino-phenoxy)-5-methyl-phenol by following the same procedure as described in Example 67: M.P.: 145-148° C. This product was analyzed by ¹H-NMR. The corresponding ¹H-NMR spectrum was consistent with the structure of the anticipated product.

### Example 68

### 1-[2-(2-Hydroxy-4-methyl-phenoxy)-phenyl]-1H-pyrazole-4-carboxylic acid ethyl ester

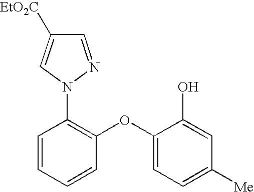

A solution of 2-(2-hydrazino-phenoxy)-5-methyl-phenol (Example 66) (100 mg, 0.434 mmol), 2-formyl-3-oxo-propionic acid ethyl ester (Bertz et al, J. Org. Chem., 1982, 47, 2216) (63 mg, 0.434 mmol) and sodium acetate (36 mg, 0.434 mmol) in ethanol (1 mL) was refluxed for 3 hrs. The reaction was evaporated under reduced pressure and the residue was purified by flash chromatography on silica column eluted with ethyl acetate/hexane (30%). The Example title compound (113 mg, 77%) was obtained as a white powder: M.P.: 118-119° C.; C₁₉H₁₈N₂O₄ (338.13): GC-MS (EI+) m/e: 338. This product was analyzed by ¹H-NMR. The corresponding ¹H-NMR spectrum was consistent with the structure of the anticipated product.

### Example 69

### 1-[4-(2-Hydroxy-4-methyl-phenoxy)-phenyl]-1H-pyrazole-4-carboxylic acid ethyl ester

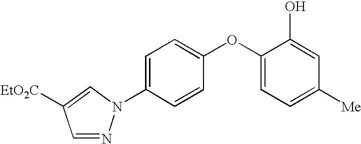

The Example title compound (160 mg, 54%) was made from 2-(4-hydrazino-phenoxy)-5-methyl-phenol (Example 67) employing the same procedure as described in Example 68: M.P.: 122-123° C. The product was analyzed by ¹H-NMR. The corresponding ¹H-NMR spectrum was consistent with the structure of the anticipated product.

### Example 70

### 1-[2-(2-Hydroxy-4-methyl-phenoxy)-phenyl]-1H-pyrazole-4-carboxylic acid

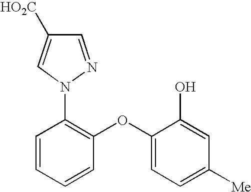

The Example title compound (59 mg, 81%) was made by hydrolysis of 1-[2-(2-hydroxy-4-methyl-phenoxy)-phenyl]-1H-pyrazole-4-carboxylic acid ethyl ester (Example 69) following the procedure described in Example 65: M.P.: 219-221° C. The product was analyzed by ¹H-NMR. The corresponding ¹H-NMR spectrum was consistent with the structure of the anticipated product.

### Example 71

### 1-[4-(2-Hydroxy-4-methyl-phenoxy)-phenyl]-1H-pyrazole-4-carboxylic acid

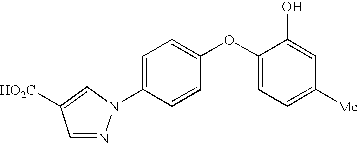

The Example title compound (90 mg, 82%) was made by hydrolysis of 1-[4-(2-hydroxy-4-methyl-phenoxy)-phenyl]-1H-pyrazole-4-carboxylic acid ethyl ester (Example 68) following the procedure described in Example 65: M.P.: 231-233° C. The product was analyzed by ¹H-NMR. The corresponding ¹H-NMR spectrum was consistent with the structure of the anticipated product.

### Example 72

### 5-Methyl-2-(2-[1,2,4]triazol-1-yl-phenoxy)-phenol

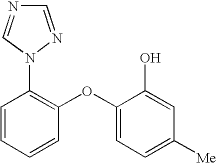

A solution of 2-(2-hydrazino-phenoxy)-5-methyl-phenol (Example 66) (1.65 mg, 0.72 mmol) and 1,3,5-triazine (61 mg, 0.75 mmol) in ethanol (1 mL) was refluxed overnight. The reaction mixture was evaporated under reduced pressure and the residue was purified on silica column eluted with MeOH/DCM (5%), affording the Example title compound (133 mg, 69%) as a white powder: M.P.: 132-133° C. The product was analyzed by ¹H-NMR. The corresponding ¹H-NMR spectrum was consistent with the structure of the anticipated product.

### Example 73

### 5-Methyl-2-(4-[1,2,4]triazol-1-yl-phenoxy)-phenol

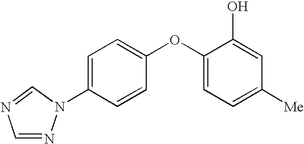

The Example title compound (118 mg, 61%) was obtained from 2-(4-hydrazino-phenoxy)-5-methyl-phenol (Example 67) following the procedure described in Example 72: M.P.: 107-109° C.

### Example 74

### 5-Amino-1-[2-(2-hydroxy-4-methyl-phenoxy)-phenyl]-1H-pyrazole-4-carboxylic acid ethyl ester

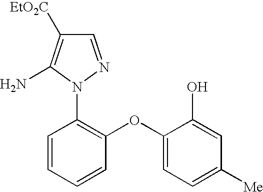

A suspension of 2-(2-hydrazino-phenoxy)-5-methyl-phenol (Example 66) (115 mg, 0.50 mmol) and 2-cyano-3-ethoxy-acrylic acid ethyl ester (104 mg, 0.6 mmol) in acetonitrile (1 mL) was refluxed overnight. The reaction mixture was evaporated and the residue was subjected to flash chromatography on silica column eluted with AcOEt/hexane (30%), giving the Example title compound (110 mg, 62%) as a white powder: 75-77° C.

### Example 75

### 5-Amino-1-[4-(2-hydroxy-4-methyl-phenoxy)-phenyl]-1H-pyrazole-4-carboxylic acid ethyl ester

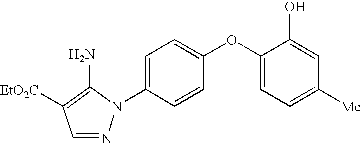

The Example title compound (92 mg, 35%) was obtained from 2-(4-hydrazino-phenoxy)-5-methyl-phenol (Example 68) following the procedure described in Example 74: M.P.: 142-144° C.

### Example 76

### 2-[2-(5-Amino-3-methylsulfanyl-[1,2,4]triazol-1-yl)-phenoxy]-5-methyl-phenol

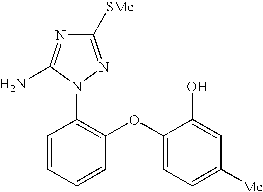

A suspension of 2-(2-hydrazino-phenoxy)-5-methyl-phenol (Example 66) (115 mg, 0.50 mmol) and 90% dimethyl cyanodithioiminocarbonate (98 mg, 0.6 mmol) in acetonitrile (1 mL) was refluxed overnight. The reaction mixture was evaporated and the residue was subjected to flash chromatography on silica column eluted with AcOEt/hexane (30%), giving the Example title compound (63 mg, 38%) as a yellowish powder: M.P.: 41-42° C.

Minimum Inhibition Concentration (MIC), Broth Dilution Method:

The compounds of the present invention were tested against an assortment of Gram positive and Gram negative organisms using standard microtitration techniques well known to those skilled in the art. Cultures of bacteria are initially brought up from the freezer by streaking a loopful onto agar plates under the appropriate conditions. For instance bacterial stocks are streaked onto chocolate agar and then incubated for 18 hours at 35-37° C. in a 5% CO₂ incubator. Five to ten colonies are picked from the chocolate agar plate for subculture to Brain-Heart infusion (BHI) broth, Mueller Hinton broth, or BHI containing 4% serum and incubated under the appropriate conditions. The ability of the test compound to act as an antimicrobial is determined by the ability to inhibit bacterial growth in vitro. The optical density of the culture of organisms in the presence of an active compound is compared to the optical density of the same organism untreated. The activity of the compounds is described as either negative or the lowest concentration inhibiting growth (Minimum Inhibitory Concentration [MIC]).

The activity of selected compounds of this invention against representative Gram positive and Gram negative bacteria are shown in the following Table 1.

TABLE 1
In Vitro Activity of Selected Examples of
the Invention Against Bacteria

| | MIC (μg/mL) | | | |
|---|---|---|---|---|
| | Gram-Positive Bacteria | | Gram-Negative Bacteria | |
| | S. aureus | S. pneumoniae | H. influenzae | E. coli |
| Example No. | 29213 | 11733 | 43095 | 43888 |
| 1 | 4 | 2 | 2 | 15.7 |
| 3 | 4 | 1 | 4 | 15.7 |
| 4 | 2 | 1 | 7.9 | 0.5 |
| 5 | 0.5 | 4 | 0.125 | 4 |
| 7 | 0.25 | 1 | 1 | 4 |
| 8 | 0.5 | 4 | 0.5 | 4 |
| 9 | 2 | 4 | 4 | 4 |
| 10 | 2 | 2 | 2 | 4 |
| 12 | 4 | 0.5 | 7.9 | 2 |
| 13 | 7.9 | 7.9 | 2 | 62.5 |
| 14 | 0.002 | 2 | 0.125 | 0.125 |
| 16 | 2 | 7.9 | 31.3 | 31.3 |
| 18 | 0.125 | 0.25 | 4 | 7.9 |
| 19 | 0.125 | 2 | 2 | 4 |
| 20 | 0.25 | 4 | 4 | 2 |
| 22 | 12.5 | 25 | >25 | >25 |
| 23 | 1.6 | 12.5 | >25 | 25 |
| 24 | 0.8 | 12.5 | 12.5 | 6.2 |
| 30 | 4 | 62.5 | 31.3 | 0.5 |
| 37 | 0.125 | 8 | 0.5 | 0.5 |
| 39 | 0.5 | >32 | 8 | 4 |
| 40 | 4 | 16 | 4 | 8 |
| 42 | 1 | >32 | 1 | 1 |
| 43 | 0.5 | 15.7 | 4 | 0.5 |
| 44 | 0.5 | >32 | 2 | 2 |
| 45 | 0.5 | 1 | 4 | 1 |
| 51 | 0.4 | >25 | 3.1 | 6.2 |
| 54 | 0.05 | 3.1 | 0.2 | 0.2 |
| 57 | 0.05 | 6.2 | 0.05 | 0.05 |
| 66 | 0.1 | 3.1 | 0.1 | 0.8 |
| 67 | 0.1 | 6.2 | 0.1 | 0.4 |
| 69 | 0.2 | >25 | >25 | 1.6 |
| 71 | 3.1 | 12.5 | 25 | >25 |

While the preferred embodiments of the invention have been illustrated and described, it will be appreciated that various changes can be made therein without departing from the spirit and scope of the invention.

What is claimed is:

1. A compound of Formula I and Formula II or a pharmaceutically acceptable salt thereof, 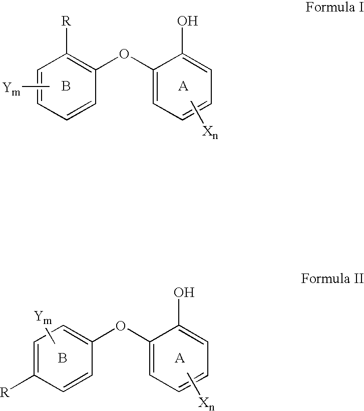
wherein,
X and Y are each F, Cl, Br, I, CN, OH, NH₂, NO₂, CONH₂, SO₂NH₂, CHO, CH(NOMe), methyl, ethyl, n-propyl, n-butyl, cyclopropyl, cycloproylmethyl and CF₃; and
m and n are 0, 1, 2 and 3.
R is C(NH)NH₂, C(NOH)NH₂, C(NNH₂)NH₂, C(O)NHOH, NHNH₂, NHC(O)H, NHC(NH)NH₂, NHSO₂Me, a heterocyclic group chosen from
(a) Substituted furanyl: 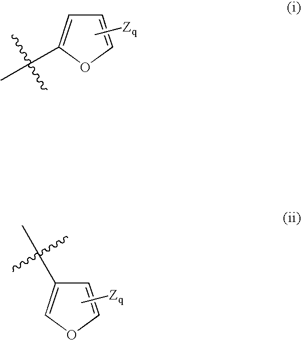
(b) Subtituted thiophenyl: 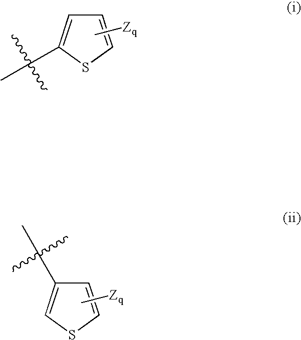
(c) Substituted pyrrolyl: 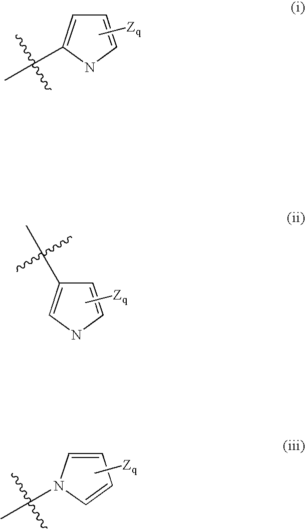
(d) Substituted isoxazolyl 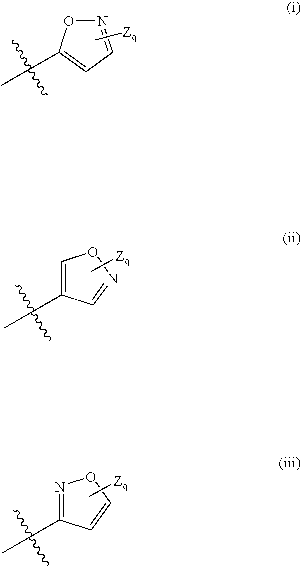
(e) Substituted isothiazolyl 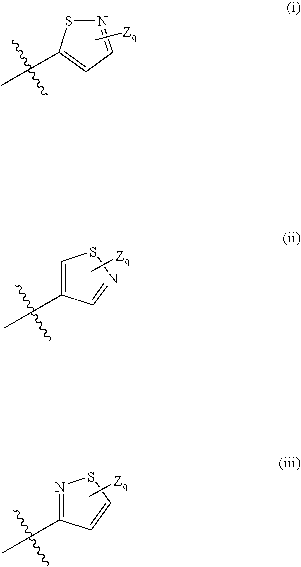
(f) Substituted pyrazolyl 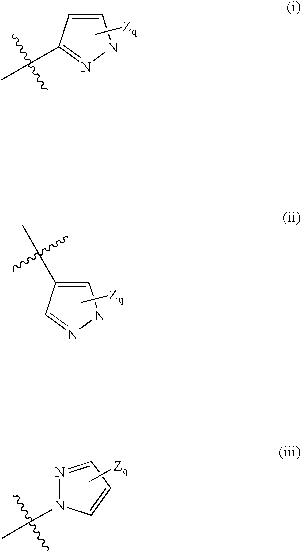
(g) Substituted oxazolyl 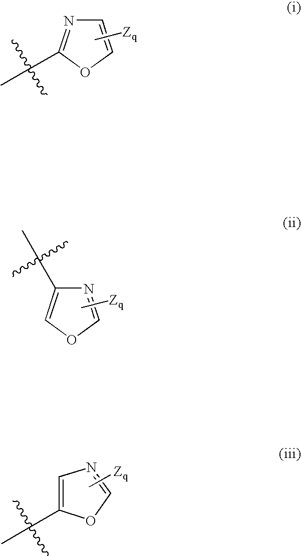
(h) Substituted thiazolyl 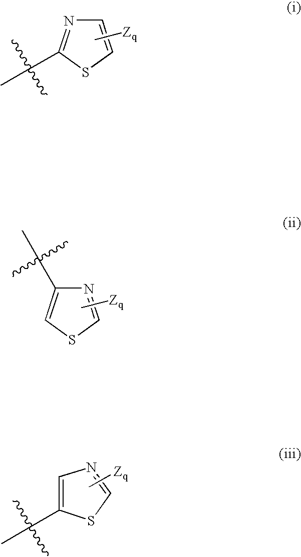
(i) Substituted imidazolyl 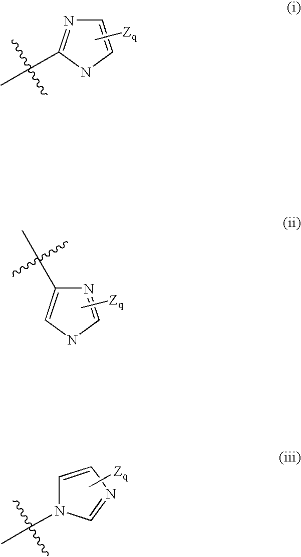
(j) Substituted 1H-[1,2,3]triazolyl 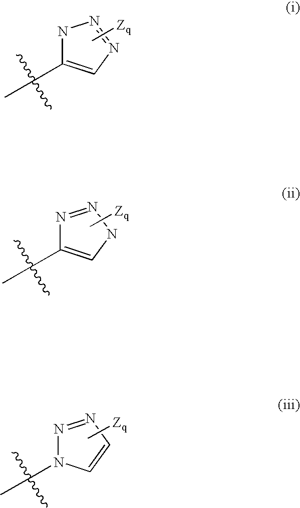
(k) Substituted 4H-[1,2,4]triazolyl 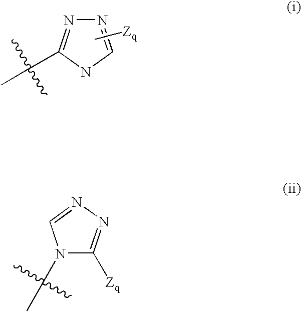
(l) Substituted 1H-[1,2,4]triazolyl 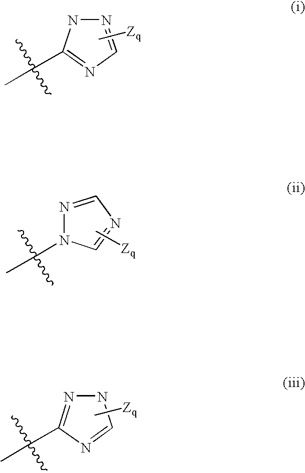
(m) Substituted [1,3,4]oxadiazolyl 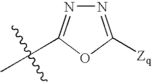
(n) Substituted [1,3,4]thiadiazolyl 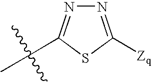
(o) Substituted [1,2,4]oxadiazolyl 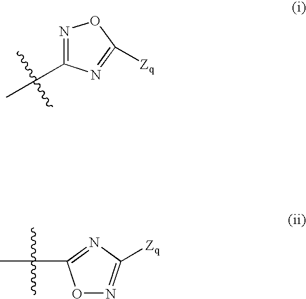
(p) 1H-tetrazol-5-yl (i) or 2H-tetrazol-5-yl (ii) 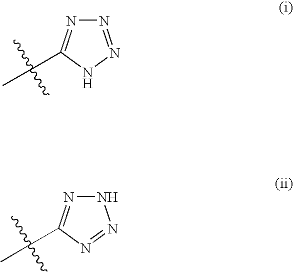
(q) 1H-tetrazol-1-yl 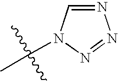
(r) 5-oxo-4H-[1,2,4]oxadiazol-3-yl 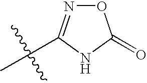
(s) Substituted thiazolidin-2-yl and [1,3]thiazinan-2-yl 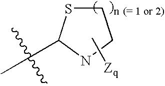
(t) Substituted 4,5-dihydro-thiazol-2-yl and 5,6-dihydro-4H-[1,3]thiazin-2-yl 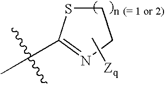
(u) Substituted pyridazinyl 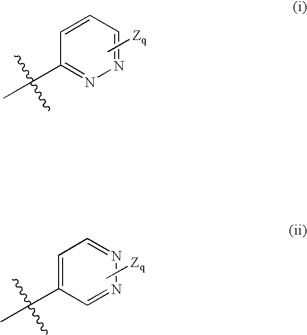
wherein Z is OH, NH₂, NHAc, Me, Et, CHO, F, Cl, CN, CH₂OH, CO₂H, CONH₂, CO₂Me, or CO₂Et; and
q is 0, 1, 2, 3.

2. The compound of claim 1, wherein
X and Y are independently F, Cl, Br, CN, methyl, ethyl, cyclopropyl, cyclopropylmethyl, CF₃, OH, NH₂, NO₂, CHO, SO₂NH₂; and
m and n are 0, 1, 2 and 3.
R is C(NH)NH₂, C(NOH)NH₂, C(NNH₂)NH₂, NHNH₂, NHC(O)H, NHC(NH)NH₂, NHSO₂Me, a heterocyclic group chosen from structural formula (a)-(i) and (ii), (b)-(i) and (ii), (c)-(i) and (iii), (f)-(i) and (iii), (i)-(i), (ii) and (iii), (j)-(i), (ii) and (iii), (k)-(i) and (ii), (l)-(i), (ii), and (iii), (m), (n), (O)-(i) and (ii), (p)-(i) and (ii), (q), (r), (s), (t) and (u), wherein Z is OH, NH₂, NHAc, Me, Et, CHO, F, Cl, CN, CH₂OH, CO₂H, CONH₂, CO₂Me, or CO₂Et; and
q is 0, 1, 2, 3.

3. The compound of claim 2, wherein
R is C(NH)NH₂, C(NOH)NH₂, C(NNH₂)NH₂, NHNH₂, NHC(O)H, NHSO₂Me, a heterocyclic group chosen from structural formula (a)-(i), (b)-(i) and (ii), (c)-(iii), (f)-(i) and (iii), (j)-(i), (ii) and (iii), (l)-(i), (ii), and (iii), (m), (n), (o)-(i), (p)-(i) and (ii), (q), (r), (s) and (t), wherein Z is independently chosen from OH, NH₂, Me, Et, CN, CO₂H, CO₂Me, or CO₂Et; and
q is 0, 1 and 2.

4. The compound of claim 3, wherein
X and Y are independently F, Cl, Br, CN, methyl, ethyl, cyclopropyl, OH, NH₂, NO₂; and
m and n are 0, 1, 2 and 3.

5. A compound selected from the group consisting of:
2-(2-Hydroxy-phenoxy)-5-(thiophen-3-yl)-phenol,
2-(2-Hydroxy-4-methyl-phenoxy)-5-(thiophen-3-yl)-phenol,
2-(2-Hydroxy-4-methyl-phenoxy)-5-(thiophen-2-yl)-phenol,
2-(2-Hydroxy-4-methyl-phenoxy)-5-(furan-2-yl)-phenol,
5-Methyl-2-[4-(thiazolidin-2-yl)-phenoxy]-phenol,
2-[4-(2-Hydroxy-4-methyl-phenoxy)-phenyl]-thiazolidine-4-carboxylic acid methyl ester,
2-[4-(2-Hydroxy-4-methyl-phenoxy)-phenyl]-thiazolidine-4-carboxylic acid,
2-[4-(2-Hydroxy-4-methyl-phenoxy)-phenyl]-[1, 3]thiazinane-4-carboxylic acid;
2-[3-Cyano-4-(2-hydroxy-4-methyl-phenoxy)-phenyl]-thiazolidine-4-carboxylic acid,
2-[3-Cyano-4-(2-hydroxy-4-methyl-phenoxy)-phenyl]-[1, 3]thiazinane-4-carboxylic acid,
5-Methyl-2-[4-(pyrrol-1-yl)-phenoxy]-phenol,
5-(Furan-2-yl)-2-(4-fluoro-2-hydroxy-phenoxy)-phenol,
5-Methyl-2-[4-(1H-tetrazol-5-yl)-phenoxy]-phenol and 5-methyl-2-[4-(2H-tetrazol-5-yl)-phenoxy]-phenol,
5-Methyl-2-[2-(1H-tetrazol-5-yl)-phenoxy]-phenol and 5-methyl-2-[2-(2H-tetrazol-5-yl)-phenoxy]-phenol,
5-Chloro-2-[2-(1H-tetrazol-5-yl)-phenoxy]-phenol and 5-chloro-2-[2-(2H-tetrazol-5-yl)-phenoxy]-phenol,
5-Methyl-2-[4-nitro-2-(1H-tetrazol-5-yl)-phenoxy]-phenol and 5-methyl-2-[4-nitro-2-(2H-tetrazol-5-yl)-phenoxy]-phenol,
5-Fluoro-2-[2-(1H-tetrazol-5-yl)-phenoxy]-phenol and 5-fluoro-2-[2-(2H-tetrazol-5-yl)-phenoxy]-phenol,
2-[4-Chloro-2-(1H-tetrazol-5-yl)-phenoxy]-5-fluoro-phenol and 2-[4-chloro-2-(2H-tetrazol-5-yl)-phenoxy]-5-fluoro-phenol,
5-chloro-2-[4-Chloro-2-(1H-tetrazol-5-yl)-phenoxy]-phenol and 5-chloro-2-[4-chloro-2-(2H-tetrazol-5-yl)-phenoxy]-phenol,
2-[2-(1H-Tetrazol-5-yl)-phenoxy]-phenol and 2-[2-(2H-tetrazol-5-yl)-phenoxy]-phenol;
4-Bromo-2-[2-(1H-tetrazol-5-yl)-phenoxy]-phenol1 and 4-bromo-2-[2-(2H-tetrazol-5-yl)-phenoxy]-phenol;
2-[2-(5-Amino-[1,3,4]thiadiazol-2-yl)-phenoxy]-5-fluoro-phenol;
2-[4-(5-Amino-[1,3,4]thiadiazol-2-yl)-phenoxy]-5-methyl-phenol,
2-[4-(5-Amino-[1,3,4]oxadiazol-2-yl)-phenoxy]-5-methyl-phenol,
2-(4-Fluoro-2-hydroxy)-N-hydroxy-benzamidine,
N-Hydroxy-2-(2-hydroxy-4-methyl-phenoxy)-benzamidine,
5-Chloro-2-(4-fluoro-2-hydroxy)-N-hydroxy-benzamidine,
N-Hydroxy-2-(2-hydroxy-4-methyl-phenoxy)-5-nitro-benzamidine,
5-Chloro-2-(4-chloro-2-hydroxy-phenoxy)-N-hydroxy-benzamidine,
N-Hydroxy-4-(2-hydroxy-4-methyl-phenoxy)-benzamidine,
3-[2-(4-Fluoro-2-hydroxy)-phenyl]-[1,2,4]oxadiazole-5-carboxylic acid ethyl ester,
3-[2-(2-Hydroxy-4-methyl-phenoxy)-phenyl]-[1,2,4]oxadiazole-5-carboxylic acid ethyl ester,
3-[5-Chloro-2-(4-fluoro-2-hydroxy-phenoxy)-phenyl]-[1,2,4]oxadiazole-5-carboxylic acid ethyl ester,
3-[2-(2-Hydroxy-4-methyl-phenoxy)-5-nitro-phenyl]-[1,2,4]oxadiazole-5-carboxylic acid ethyl ester,
3-[4-(2-Hydroxy-4-methyl-phenoxy)-phenyl]-[1,2,4]oxadiazole-5-carboxylic acid ethyl ester,
3-[2-(4-Fluoro-2-hydroxy-phenoxy)-phenyl]-[1,2,4]oxadiazole-5-carboxylic acid,
3-[2-(2-Hydroxy-4-methyl-phenoxy)-phenyl]-[1,2,4]oxadiazole-5-carboxylic acid,
3-[5-Chloro-2-(4-fluoro-2-hydroxy-phenoxy)-phenyl]-[1,2,4]oxadiazole-5-carboxylic acid,
3-[2-(2-Hydroxy-4-methyl-phenoxy)-5-nitro-phenyl]-[1,2,4]oxadiazole-5-carboxylic acid,
3-[4-(2-Hydroxy-4-methyl-phenoxy)-phenyl]-[1,2,4]oxadiazole-5-carboxylic acid,
5-Methyl-2-(4-nitro-2-[1,2,4]oxadiazol-3-yl-phenoxy)-phenol,
5-Fluoro-2-(2-[1,2,4]oxadiazol-3-yl-phenoxy)-phenol,
5-Methyl-2-(2-[1,2,4]oxadiazol-3-yl-phenoxy)-phenol,
2-(4-Chloro-2-[1,2,4]oxadiazol-3-yl-phenoxy)-5-fluoro-phenol,
5-Methyl-2-(4-[1,2,4]oxadiazol-3-yl-phenoxy)-phenol,
3-[2-(2-Hydroxy-4-methyl-phenoxy)-phenyl]-4H-[1,2,4]oxadiazol-5-one;
3-[4-(2-Hydroxy-4-methyl-phenoxy)-phenyl]-4H-[1,2,4]oxadiazol-5-one;
2-(4-Chloro-2-hydroxy-phenoxy)-N-hydroxy-benzamidine;
N-Hydroxy-4-(2-hydroxy-phenoxy)-3-nitro-benzamidine;
4-(4-Chloro-2-hydroxy-phenoxy)-N-hydroxy-3-nitro-benzamidine,
3-[2-(4-Chloro-2-hydroxy-phenoxy)-phenyl]-[1,2,4]oxadiazole-5-carboxylic acid ethyl ester,
3-[4-(2-Hydroxy-phenoxy)-3-nitro-phenyl]-[1,2,4]oxadiazole-5-carboxylic acid ethyl ester,
3-[4-(4-Chloro-2-hydroxy-phenoxy)-3-nitro-phenyl]-[1,2,4]oxadiazole-5-carboxylic acid ethyl ester,
3-[2-(4-Chloro-2-hydroxy-phenoxy)-phenyl]-[1,2,4]oxadiazole-5-carboxylic acid,
3-[4-(2-Hydroxy-phenoxy)-3-nitro-phenyl]-[1,2,4]oxadiazole-5-carboxylic acid,
3-[4-(4-Chloro-2-hydroxy-phenoxy)-3-nitro-phenyl]-[1,2,4]oxadiazole-5-carboxylic acid,
5-Chloro-2-(2-[1,2,4]oxadiazol-3-yl-phenoxy)-phenol,
5-Methyl-2-(2-tetrazol-1-yl-phenoxy)-phenol,
5-Methyl-2-(4-tetrazol-1-yl-phenoxy)-phenol,
N-Amino-4-(2-hydroxy-4-methyl-phenoxy)-benzamidine,
N-Amino-2-(2-hydroxy-4-methyl-phenoxy)-benzamidine,
1-[2-(2-Hydroxy-4-methyl-phenoxy)-phenyl]-1H-[1,2,3]triazole-4-carboxylic acid ethyl ester,
1-[4-(2-Hydroxy-4-methyl-phenoxy)-phenyl]-1H-[1,2,3]triazole-4-carboxylic acid ethyl ester,
1-[2-(2-Hydroxy-4-methyl-phenoxy)-phenyl]-1H-[1,2,3]triazole-4-carboxylic acid,
1-[4-(2-Hydroxy-4-methyl-phenoxy)-phenyl]-1H-[1,2,3]triazole-4-carboxylic acid,
2-(2-Hydrazino-phenoxy)-5-methyl-phenol,
2-(4-Hydrazino-phenoxy)-5-methyl-phenol,
1-[2-(2-Hydroxy-4-methyl-phenoxy)-phenyl]-1H-pyrazole-4-carboxylic acid ethyl ester,
1-[4-(2-Hydroxy-4-methyl-phenoxy)-phenyl]-1H-pyrazole-4-carboxylic acid ethyl ester,
1-[2-(2-Hydroxy-4-methyl-phenoxy)-phenyl]-1H-pyrazole-4-carboxylic acid,
1-[4-(2-Hydroxy-4-methyl-phenoxy)-phenyl]-1H-pyrazole-4-carboxylic acid,
5-Methyl-2-(2-[1,2,4]triazol-1-yl-phenoxy)-phenol,
5-Methyl-2-(4-[1,2,4]triazol-1-yl-phenoxy)-phenol,
5-Amino-1-[2-(2-hydroxy-4-methyl-phenoxy)-phenyl]-1H-pyrazole-4-carboxylic acid ethyl ester,
5-Amino-1-[4-(2-hydroxy-4-methyl-phenoxy)-phenyl]-1H-pyrazole-4-carboxylic acid ethyl ester, and
2-[2-(5-Amino-3-methylsulfanyl-[1,2,4]triazol-1-yl)-phenoxy]-5-methyl-phenol.

6. A compound selected from the group consisting of:
2-(2-Hydroxy-phenoxy)-5-(thiophen-3-yl)-phenol,
2-(2-Hydroxy-4-methyl-phenoxy)-5-(thiophen-3-yl)-phenol,
2-(2-Hydroxy-4-methyl-phenoxy)-5-(thiophen-2-yl)-phenol,
2-(2-Hydroxy-4-methyl-phenoxy)-5-(furan-2-yl)-phenol,
5-Methyl-2-[4-(thiazolidin-2-yl)-phenoxy]-phenol,
2-[4-(2-Hydroxy-4-methyl-phenoxy)-phenyl]-thiazolidine-4-carboxylic acid methyl ester,
2-[4-(2-Hydroxy-4-methyl-phenoxy)-phenyl]-thiazolidine-4-carboxylic acid,
2-[4-(2-Hydroxy-4-methyl-phenoxy)-phenyl]-[1, 3]thiazinane-4-carboxylic acid,
2-[3-Cyano-4-(2-hydroxy-4-methyl-phenoxy)-phenyl]-thiazolidine-4-carboxylic acid,
2-[3-Cyano-4-(2-hydroxy-4-methyl-phenoxy)-phenyl]-[1, 3]thiazinane-4-carboxylic acid,
5-(Furan-2-yl)-2-(4-fluoro-2-hydroxy-phenoxy)-phenol,
5-Methyl-2-[4-(1H-tetrazol-5-yl)-phenoxy]-phenol and 5-methyl-2-[4-(2H-tetrazol-5-yl)-phenoxy]-phenol,
5-Methyl-2-[2-(1H-tetrazol-5-yl)-phenoxy]-phenol and 5-methyl-2-[2-(2H-tetrazol-5-yl)-phenoxy]-phenol,
5-Chloro-2-[2-(1H-tetrazol-5-yl)-phenoxy]-phenol and 5-chloro-2-[2-(2H-tetrazol-5-yl)-phenoxy]-phenol,
5-Methyl-2-[4-nitro-2-(1H-tetrazol-5-yl)-phenoxy]-phenol and 5-methyl-2-[4-nitro-2-(2H-tetrazol-5-yl)-phenoxy]-phenol,
5-Fluoro-2-[2-(1H-tetrazol-5-yl)-phenoxy]-phenol and 5-fluoro-2-[2-(2H-tetrazol-5-yl)-phenoxy]-phenol,
2-[4-Chloro-2-(1H-tetrazol-5-yl)-phenoxy]-5-fluoro-phenol and 2-[4-chloro-2-(2H-tetrazol-5-yl)-phenoxy]-5-fluoro-phenol,
5-chloro-2-[4-Chloro-2-(1H-tetrazol-5-yl)-phenoxy]-phenol and 5-chloro-2-[4-chloro-2-(2H-tetrazol-5-yl)-phenoxy]-phenol,
2-(4-Fluoro-2-hydroxy)-N-hydroxy-benzamidine,
N-Hydroxy-2-(2-hydroxy-4-methyl-phenoxy)-benzamidine,
5-Chloro-2-(4-fluoro-2-hydroxy)-N-hydroxy-benzamidine,
N-Hydroxy-2-(2-hydroxy-4-methyl-phenoxy)-5-nitro-benzamidine,
5-Chloro-2-(4-chloro-2-hydroxy-phenoxy)-N-hydroxy-benzamidine,
N-Hydroxy-4-(2-hydroxy-4-methyl-phenoxy)-benzamidine,
3-[2-(4-Fluoro-2-hydroxy)-phenyl]-[1,2,4]oxadiazole-5-carboxylic acid ethyl ester,
3-[2-(2-Hydroxy-4-methyl-phenoxy)-phenyl]-[1,2,4]oxadiazole-5-carboxylic acid ethyl ester,
3-[5-Chloro-2-(4-fluoro-2-hydroxy-phenoxy)-phenyl]-[1,2,4]oxadiazole-5-carboxylic acid ethyl ester,
3-[2-(2-Hydroxy-4-methyl-phenoxy)-5-nitro-phenyl]-[1,2,4]oxadiazole-5-carboxylic acid ethyl ester,
3-[4-(2-Hydroxy-4-methyl-phenoxy)-phenyl]-[1,2,4]oxadiazole-5-carboxylic acid ethyl ester,
3-[2-(4-Fluoro-2-hydroxy-phenoxy)-phenyl]-[1,2,4]oxadiazole-5-carboxylic acid,
3-[2-(2-Hydroxy-4-methyl-phenoxy)-phenyl]-[1,2,4]oxadiazole-5-carboxylic acid,
3-[5-Chloro-2-(4-fluoro-2-hydroxy-phenoxy)-phenyl]-[1,2,4]oxadiazole-5-carboxylic acid,
3-[2-(2-Hydroxy-4-methyl-phenoxy)-5-nitro-phenyl]-[1,2,4]oxadiazole-5-carboxylic acid,
3-[4-(2-Hydroxy-4-methyl-phenoxy)-phenyl]-[1,2,4]oxadiazole-5-carboxylic acid,
5-Methyl-2-(4-nitro-2-[1,2,4]oxadiazol-3-yl-phenoxy)-phenol,
5-Fluoro-2-(2-[1,2,4]oxadiazol-3-yl-phenoxy)-phenol,
5-Methyl-2-(2-[1,2,4]oxadiazol-3-yl-phenoxy)-phenol,
2-(4-Chloro-2-[1,2,4]oxadiazol-3-yl-phenoxy)-5-fluoro-phenol,
5-Methyl-2-(4-[1,2,4]oxadiazol-3-yl-phenoxy)-phenol,
2-(4-Chloro-2-hydroxy-phenoxy)-N-hydroxy-benzamidine,
4-(4-Chloro-2-hydroxy-phenoxy)-N-hydroxy-3-nitro-benzamidine,
3-[2-(4-Chloro-2-hydroxy-phenoxy)-phenyl]-[1,2,4]oxadiazole-5-carboxylic acid ethyl ester,
3-[4-(2-Hydroxy-phenoxy)-3-nitro-phenyl]-[1,2,4]oxadiazole-5-carboxylic acid ethyl ester,
3-[4-(4-Chloro-2-hydroxy-phenoxy)-3-nitro-phenyl]-[1,2,4]oxadiazole-5-carboxylic acid ethyl ester,
3-[2-(4-Chloro-2-hydroxy-phenoxy)-phenyl]-[1,2,4]oxadiazole-5-carboxylic acid,
3-[4-(2-Hydroxy-phenoxy)-3-nitro-phenyl]-[1,2,4]oxadiazole-5-carboxylic acid,
3-[4-(4-Chloro-2-hydroxy-phenoxy)-3-nitro-phenyl]-[1,2,4]oxadiazole-5-carboxylic acid,
5-Chloro-2-(2-[1,2,4]oxadiazol-3-yl-phenoxy)-phenol,
5-Methyl-2-(2-tetrazol-1-yl-phenoxy)-phenol,
5-Methyl-2-(4-tetrazol-1-yl-phenoxy)-phenol,
N-Amino-4-(2-hydroxy-4-methyl-phenoxy)-benzamidine,
N-Amino-2-(2-hydroxy-4-methyl-phenoxy)-benzamidine,
1-[2-(2-Hydroxy-4-methyl-phenoxy)-phenyl]-1H-[1,2,3]triazole-4-carboxylic acid ethyl ester,
1-[4-(2-Hydroxy-4-methyl-phenoxy)-phenyl]-1H-[1,2,3]triazole-4-carboxylic acid ethyl ester,
1-[2-(2-Hydroxy-4-methyl-phenoxy)-phenyl]-1H-[1,2,3]triazole-4-carboxylic acid,
1-[4-(2-Hydroxy-4-methyl-phenoxy)-phenyl]-1H-[1,2,3]triazole-4-carboxylic acid,
2-(2-Hydrazino-phenoxy)-5-methyl-phenol,
2-(4-Hydrazino-phenoxy)-5-methyl-phenol,
1-[2-(2-Hydroxy-4-methyl-phenoxy)-phenyl]-1H-pyrazole-4-carboxylic acid ethyl ester,
1-[4-(2-Hydroxy-4-methyl-phenoxy)-phenyl]-1H-pyrazole-4-carboxylic acid ethyl ester,
1-[2-(2-Hydroxy-4-methyl-phenoxy)-phenyl]-1H-pyrazole-4-carboxylic acid,
1-[4-(2-Hydroxy-4-methyl-phenoxy)-phenyl]-1H-pyrazole-4-carboxylic acid,
5-Methyl-2-(2-[1,2,4]triazol-1-yl-phenoxy)-phenol, and
5-Methyl-2-(4-[1,2,4]triazol-1-yl-phenoxy)-phenol.

7. The compounds of claim 1 which are in the form of a prodrug selected from the group consisting of compounds wherein hydroxyl, amine, or sulfhydroxyl groups are bonded to any group that, when administered to an animal, cleave to form a free hydroxyl, amino, or sulfhydroxyl group, respectively.

8. The compounds of claim 1 which are in the form of a prodrug selected from the group consisting of acetate, formate, benzoate and phosphate ester derivatives of hydroxyl functional groups, and acetyl and benzoyl derivatives of amine functional groups.

9. The compounds of claim 1, wherein the compounds comprise tautomeric forms, geometric isomers, enantiomers and diastereomers.

10. The compounds of claim 1, wherein the pharmaceutically acceptable salt thereof is an acid addition salt wherein the acid is selected from the group consisting of hydrochloric, sulfuric, phosphoric, acetic, citric, oxalic, malonic, salicyclic, malic, gluconic, fumaric, succinic, ascorbic, maleic, and methanesulfonic acid; or a base salt formed with alkali and alkaline earth metals or organic amines.

11. A composition comprising the following compounds of Formula 1 and Formula 2, or a pharmaceutically acceptable salt thereof, and a pharmaceutically acceptable carrier, 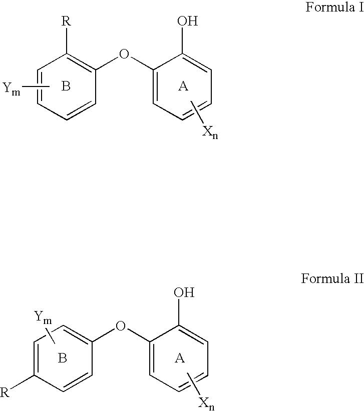
wherein,
X and Y are each F, Cl, Br, I, CN, OH, NH₂, NO₂, CONH₂, SO₂NH₂, CHO, CH(NOMe), methyl, ethyl, n-propyl, n-butyl, cyclopropyl, cycloproylmethyl and CF₃; and
m and n are 0, 1, 2 and 3.
R is C(NH)NH₂, C(NOH)NH₂, C(NNH₂)NH₂, C(O)NHOH, NHNH₂, NHC(O)H, NHC(NH)NH₂, NHSO₂Me, a heterocyclic group chosen from
(a) Substituted furanyl: 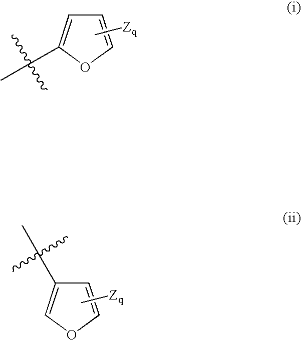
(b) Subtituted thiophenyl: 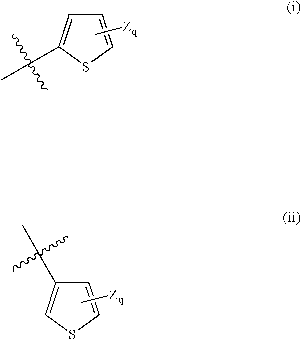
(c) Substituted pyrrolyl: 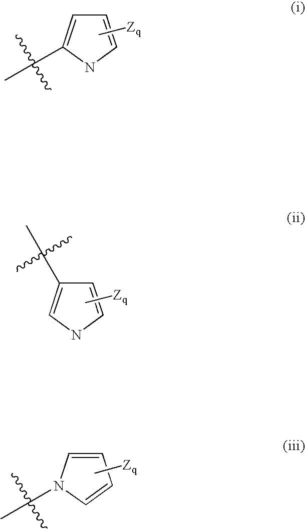
(d) Substituted isoxazolyl 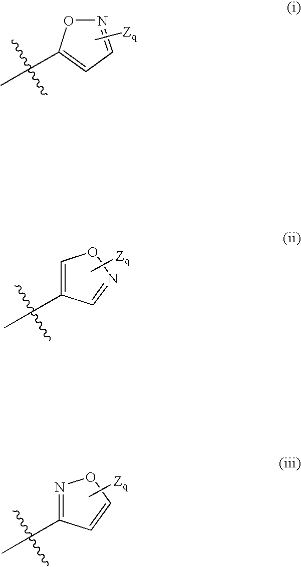
(e) Substituted isothiazolyl 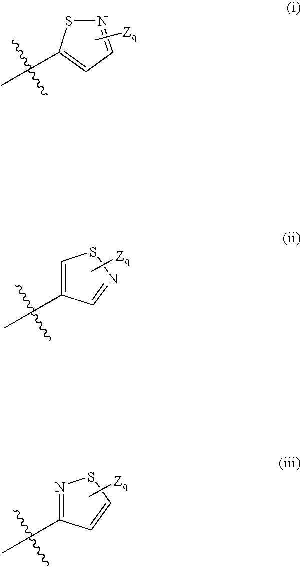
(f) Substituted pyrazolyl 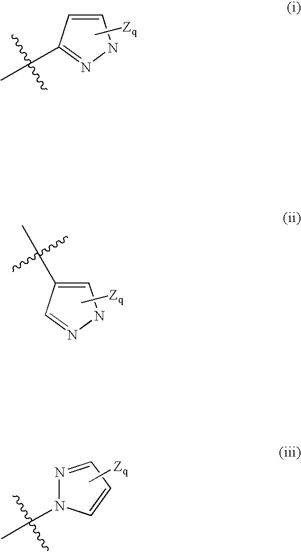
(g) Substituted oxazolyl 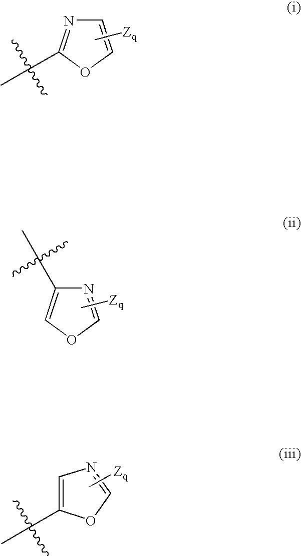
(h) Substituted thiazolyl 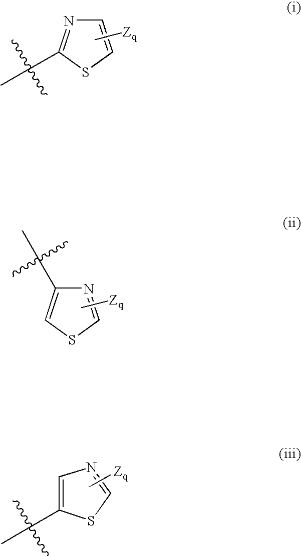
(v) Substituted imidazolyl 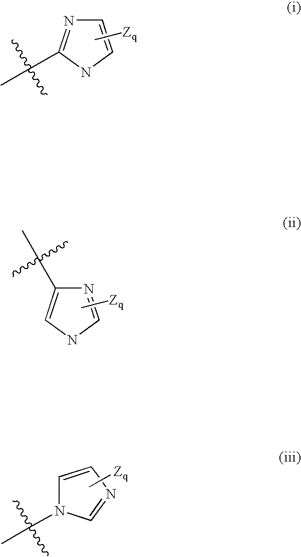
(w) Substituted 1H-[1,2,3]triazolyl 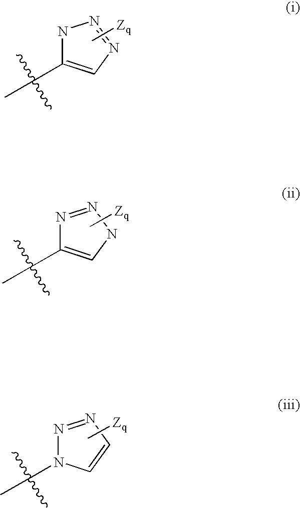
(x) Substituted 4H-[1,2,4]triazolyl 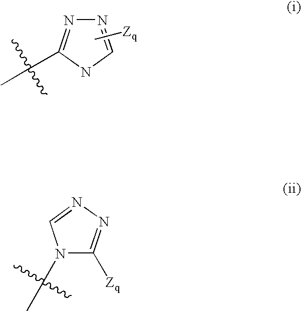
(y) Substituted 1H-[1,2,4]triazolyl 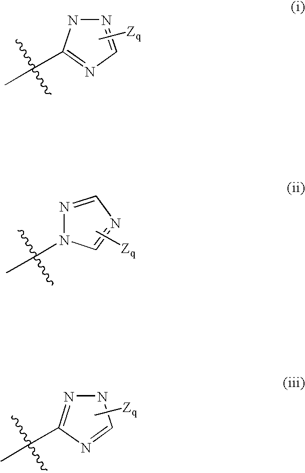
(z) Substituted [1,3,4]oxadiazolyl 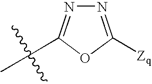
(aa) Substituted [1,3,4]thiadiazolyl 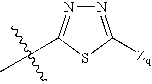
(bb) Substituted [1,2,4]oxadiazolyl 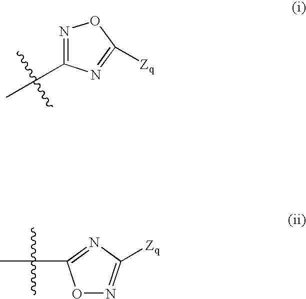
(cc) 1H-tetrazol-5-yl (i) or 2H-tetrazol-5-yl (ii) 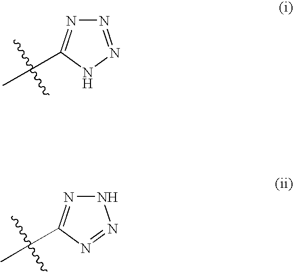
(dd) 1H-tetrazol-1-yl 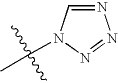
(ee) 5-oxo-4H-[1,2,4]oxadiazol-3-yl 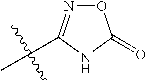
(ff) Substituted thiazolidin-2-yl and [1,3]thiazinan-2-yl 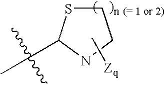
(gg) Substituted 4,5-dihydro-thiazol-2-yl and 5,6-dihydro-4H-[1,3]thiazin-2-yl 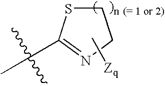
(hh) Substituted pyridazinyl 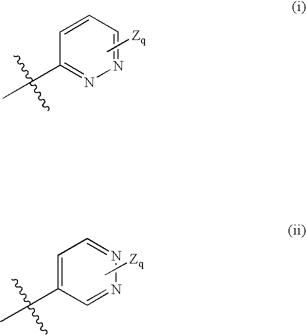
wherein Z is OH, NH₂, NHAC, Me, Et, CHO, F, Cl, CN, CH₂OH, CO₂H, CONH₂, CO₂Me, or CO₂Et; and
q is 0, 1, 2, 3.

12. The composition of claim 11, wherein the carrier is a solid material selected from the group consisting of magnesium carbonate, magnesium stearate, talc, sugar, lactose, pectin, dextrin, starch, gelatin, tragacanth, methyl cellulose, sodium carboxymethyl cellulose, a low melting wax, cocoa butter and mixtures thereof.

13. The composition of claim 11, wherein the carrier is a liquid material selected from the group consisting of water, ethyl alcohol, isopropyl alcohol, ethyl carbonate, ethyl acetate, benzyl alcohol, benzyl benzoate, propylene glycol, 1,3-butylene glycol, oils, glycerol, polyethylene glycols, fatty acid esters of sorbitan, and mixtures thereof.

14. A method of treating or preventing a disease or condition caused by or associated with a microbial infection, which method comprises the administration to an animal in need thereof a pharmaceutical composition comprising an anti-microbial amount of the following compounds of Formula 1 and Formula 2, or a pharmaceutically acceptable salt thereof, and a pharmaceutically acceptable carrier, 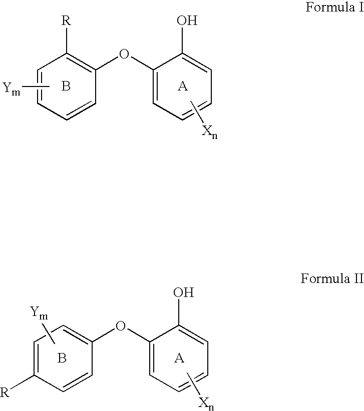
wherein,
X and Y are each F, Cl, Br, I, CN, OH, NH₂, NO₂, CONH₂, SO₂NH₂, CHO, CH(NOMe), methyl, ethyl, n-propyl, n-butyl, cyclopropyl, cycloproylmethyl and CF₃; and
m and n are 0, 1, 2 and 3.
R is C(NH)NH₂, C(NOH)NH₂, C(NNH₂)NH₂, C(O)NHOH, NHNH₂, NHC(O)H, NHC(NH)NH₂, NHSO₂Me, a heterocyclic group chosen from
(a) Substituted furanyl: 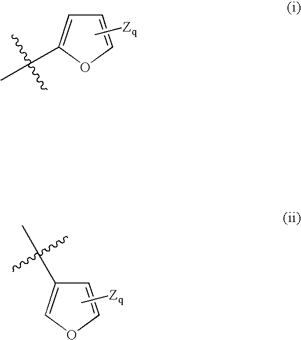
(b) Subtituted thiophenyl: 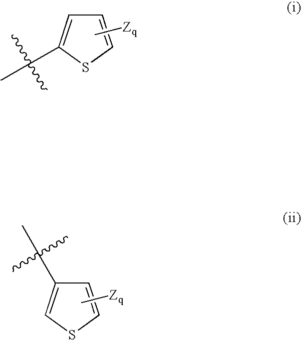
(c) Substituted pyrrolyl: 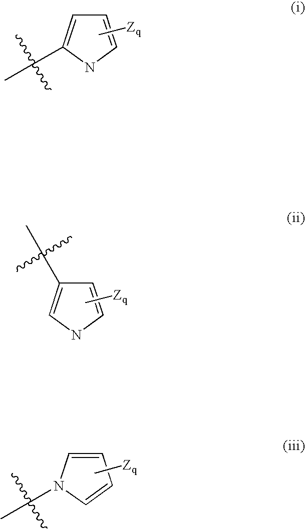
(d) Substituted isoxazolyl 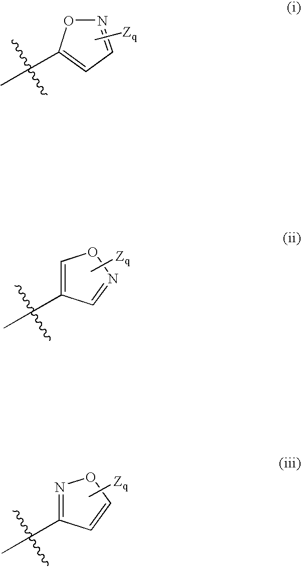
(e) Substituted isothiazolyl 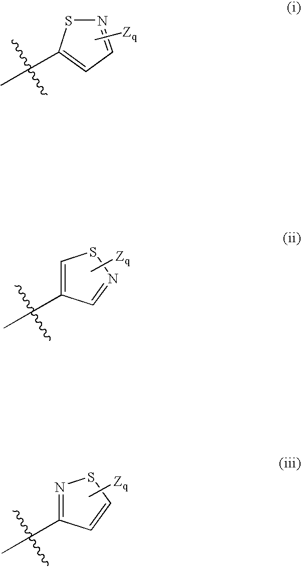
(f) Substituted pyrazolyl 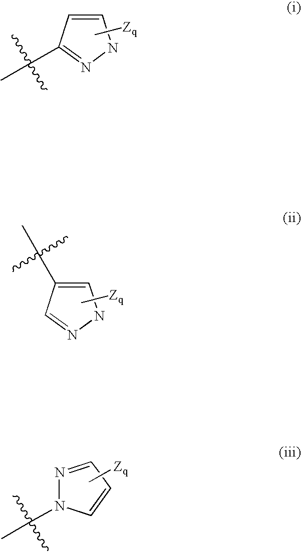
(g) Substituted oxazolyl 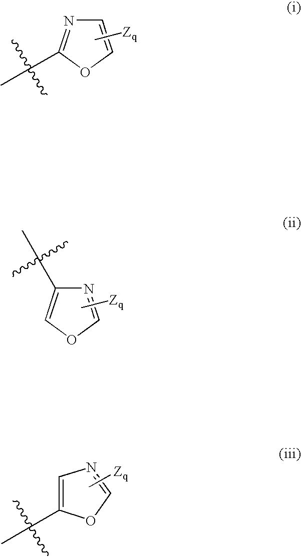
(h) Substituted thiazolyl 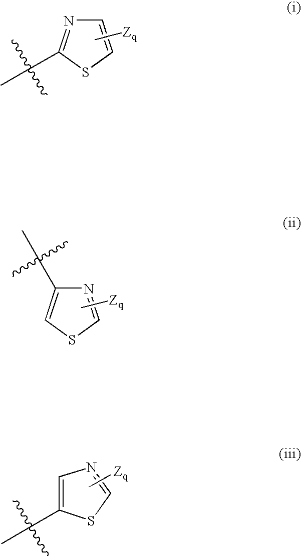
(ii) Substituted imidazolyl 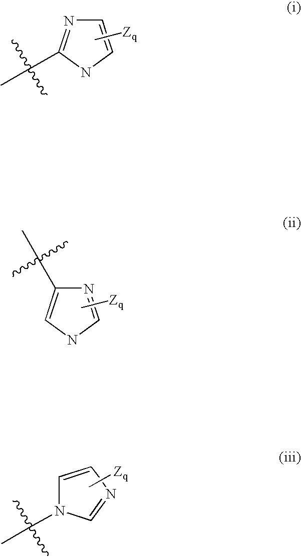
(jj) Substituted 1H-[1,2,3]triazolyl 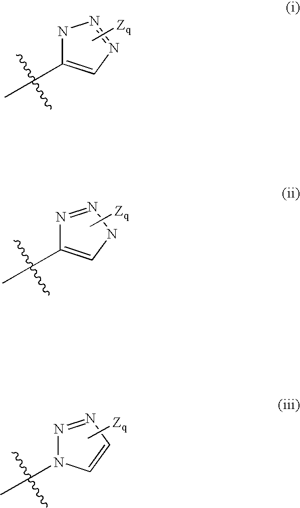
(kk) Substituted 4H-[1,2,4]triazolyl 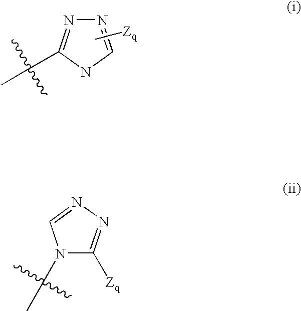
(ll) Substituted 1H-[1,2,4]triazolyl 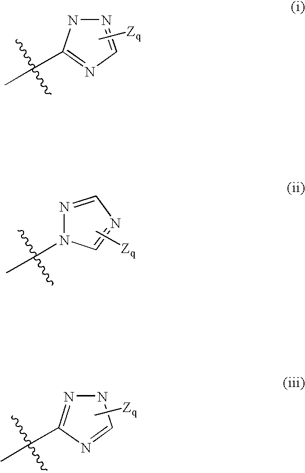
(mm) Substituted [1,3,4]oxadiazolyl 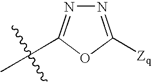
(nn) Substituted [1,3,4]thiadiazolyl 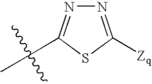
(oo) Substituted [1,2,4]oxadiazolyl 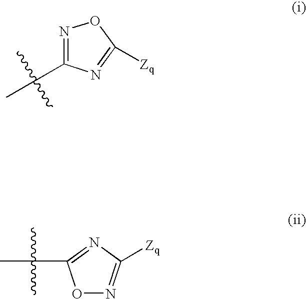
(pp) 1H-tetrazol-5-yl (i) or 2H-tetrazol-5-yl (ii) 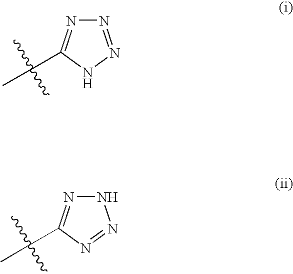
(qq) 1H-tetrazol-1-yl 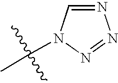
(rr) 5-oxo-4H-[1,2,4]oxadiazol-3-yl 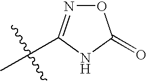
(ss) Substituted thiazolidin-2-yl and [1,3]thiazinan-2-yl 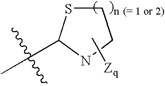
(tt) Substituted 4,5-dihydro-thiazol-2-yl and 5,6-dihydro-4H-[1,3]thiazin-2-yl 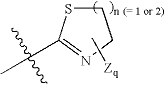
(uu) Substituted pyridazinyl 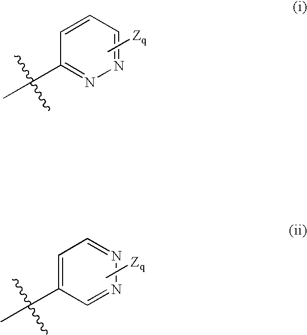
wherein Z is OH, NH₂, NHAC, Me, Et, CHO, F, Cl, CN, CH₂OH, CO₂H, CONH₂, CO₂Me, or CO₂Et; and
q is 0, 1, 2, 3.

15. The method of claim 14 wherein the composition is administered to at least one of the skin, mouth, eye, respiratory tract, urinary tract, reproductive tract, soft tissues and blood of an animal.

16. The method of claim 14 wherein the animal is a human.

17. The method of claim 14 wherein the composition is applied to the skin of an animal for topical or transdermal administration.

18. The method of claim 17, wherein the composition for topical or transdermal administration is in a form selected from the group consisting of powders, sprays, ointments, pastes, creams, lotions, gels, solutions, patches and inhalants.

19. The method of claim 14 wherein the disease or condition is caused by or associated with infection with a microbe selected from the group consisting of Streptococcus pyogenes, Staphylococcus aureus, methicillin resistant Staphylococcus aureus (“MRSA”), Staphylococcus epidermidis, Bacillus anthracis, Neisseria gonorrhoeae, Neisseria meningitidis, Mycobacteria tuberculosis, vancomycin resistant Enterococcae (“VRE”), Helicobacter pylori, Chlamydia pneumoniae, Chlamydia trachomatis, Campylobacter jejuni, Propionibacterium acnes, Pseudomonas aeruginosa, Haemophilus influenzae, Streptococcus pneumoniae, Enterococcus faecalis, Escherichia coli, Corynebacterium diphtheriae, Morazella catarrhalis and Bacillus cereus.

20. The method of claim 14 wherein the composition is administered two or more times.

21. The method of claim 14 wherein the compound is administered in a dose of about 0.0001 to about 100 mg per kilogram of body weight per day

22. The method of claim 14 wherein the compound is administered in an amount of about 0.01 to about 50 mg per kg of body weight per day.

23. The method of claim 14 wherein the compound is administered in a dose of about 0.1 to about 10 mg per kg of body weight per day

24. The method of claim 15 wherein the dose of compound administered is selected from the group consisting of 5, 10, 25, 50, 100, 125, 150, 200, 250 and 500 mg per kg of body weight per day.
